(19) United States
(12) Patent Application Publication
(10) Pub. No.: US 2011/0189161 A1
(43) Pub. Date: Aug. 4, 2011

(54) NUTRIGENOMICS METHODS AND COMPOSITIONS

(76) Inventors: Kenneth Blum, San Diego, CA (US); Roger L. Waite, San Diego, CA (US); B. William Downs, Lederach, PA (US); William J. Heaney, Huntington Beach, CA (US)

(21) Appl. No.: 13/000,623

(22) PCT Filed: Jun. 22, 2009
(86) PCT No.: PCT/US09/48074

§ 371 Date: Dec. 21, 2010

Related U.S. Application Data
(60) Provisional application No. 61/074,629, filed on Jun. 21, 2008.

(51) Int. Cl. A61K 38/48; A61K 38/02; A61K 36/41; A61K 35/32; A61P 25/24; A61P 25/22; A61P 25/00; A61P 19/02; A61K 36/28; A61K 36/16; A61K 36/258; A61K 36/67; A61K 35/56; A61K 36/84; A61K 36/38; A61K 33/24; A61K 33/10; A61K 35/60

(52) U.S. Cl. 424/94.65; 514/17.6; 514/17.5; 514/17.7; 514/18.3; 424/725; 424/548; 424/764; 424/752; 424/728; 424/734; 424/547; 424/733; 424/730; 424/655; 424/687; 424/523

(57) ABSTRACT

The present invention provides a proprietary compositions and systems to modulate genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for the purposes of customizing a subject's nutritional supplement formulation to optimize specific health outcomes. Specific to this invention the utilization of certain known polymorphic genes associated with Substance Use Disorder (SUD) are analyzed to target certain genetic anomalies that lead to a high risk and predisposition to SUD. The genotypic patterns are then utilized to provide certain nutritional customized solutions especially related to the attenuation of aberrant abuse of physician prescribed narcotic pain medication across all pain conditions. A priority GENOPROFILE is measured and directs the customization of a subsequent nutraceutical to act as a therapeutic modality. Specifically the treatment includes slow attenuation of the pain medication by incorporating orals (shakes, liquid beverages, pills, tablets, troche, ointments etc.), Intramuscular, Intravenous, intra-rectal and any form necessary to deliver a sufficient amount of an anti-craving and anti-stress nutraceutical. Moreover, the invention includes examples of novel analgesic ointments coupling Synaptamine and such analgesic and other anesthetic compounds including but not limited to Gabapentin, Ketamine, Baclofen, Ketoprofen, Amitriptyline, Lidocaine, Cyclobenzapine, Diclofenac, Menthol, Camphor and Capsaicin. The GENOPROFILE will be used to determine pain sensitivity Intolerance.

### RELATED APPLICATIONS

This application is a national stage filing of PCT patent application no. PCT/US2009/048074, filed 22 Jun. 2009, and this application hereby claims the benefit of and priority to PCT/US2009/048074, of which this application is a continuation-in-part of, and U.S. provisional patent application Ser. No. 61/074,629, filed 21 Jun. 2008, the contents of each of which are herein incorporated by reference in their entirety for any and all purposes.

### BACKGROUND OF INVENTION

Nutragenomics

It is well know that individuals respond differently to medications and certain nutraceuticals in terms of both toxicity and treatment efficacy. Potential causes for such variability in drug (nutrient) effects include the pathogenesis and severity of the disease being treated: drug (nutrient) interactions; the individual's age, nutritional status; kidney and liver function; and concomitant illnesses. Despite the potential importance of these clinical variables in determining drug/nutrient effects, it is now recognized that inherited differences in the metabolism and disposition of drugs/nutrients, and genetic variants (polymorphisms) in the targets of drug/nutrient therapy (such as receptors like the dopamine D2 receptor [DRD2]), can have even greater influence on the efficacy and toxicity of either medications or nutraceuticals.

Many genes encoding drug targets exhibit genetic polymorphism (variants), which in many cases alters their sensitivity to specific medications and/or offer specific targeted therapy.

Such examples include the following:
Asthma—Polymorphisms in Beta-adrenergic receptors (adrenalin-like) impart differential sensitivity to substances that stimulate these receptors (beta-agonists) in asthmatics.
Renal function and Blood pressure—angiotensin converting enzyme (ACE) gene polymorphisms impart differential sensitivity to inhibitors of ACE.
Cardiovascular—angiotensin 11 T1 receptor gene polymorphisms impart differential sensitivity to the substance phenylalanine and subsequent vascular reactivity.
Diabetes—polymorphisms in the sulfonyurea receptor gene imparts differential responsiveness to sulfonyurea hypoglycemic agents.
Coronary atherosclerosis—polymorphisms in the gene that controls the enzyme cholesteryl ester transfer protein impart differential efficacy of the drug pravastatin in patients with coronary disease.
Dysrthythmias—Potassium channel mutations predict drug-induced dysrythmias as an adverse effect.
Drug Metabolism—Polymorphisms in the P-450 enzymes responsible for metabolizing drugs such as caffeine and codeine impart differential clearance of these and other substances. One such an enzyme is the CYP2D6.
Breast Cancer—Trasruzumab is a drug known to target a certain genetic mutation in a protein product of the HER2/neu oncogene (which is overexpressed in breast cancers) and has been found compared to standard therapy to be superior un preventing metastatic breast cancer.
Diuretic therapy—There is a gene known as C825T involved with a second messenger G-protein {beta}3 whereas polymorphisms in this gene predict responsiveness to the anti-diuretic drug (used to treat hypertension), hydrochlorothiazide.
Lipid response—Genetic variation of the apolipoprotein constituents of the lipoprotein molecules (APOE gene locus) predicts plasma low-density lipoprotein cholesterol (LDL-C) concentrations. Interesting carrying one form of the APOE (E4) seems to be more responsive to dietary modification than carriers of E3 and or E2 forms of the same gene.
Nicotine patch—Variation of the CT and TT allele of the dopamine D2 receptor gene confirms a differential response to the nicotine patch. At the eight-year mark, 12% of women with the CT or TT allele of the dopamine D2 receptor gene who had received the patch had remained abstinent. Only 5% of women with the CC allele had maintained their non-smoking status. No difference based on genetics was noted in men.
The polymorphic CYP2D6 regulates the O-demethylation of codeine and other weak opioids to more potent metabolites with poor meta bolizers having reduced antinociception in some cases.

In the broadest terms, the interface between the nutritional environment and cellular/genetic processes is being referred to as “nutrigenomics”. While nutrigenomics in this sense seeks to provide a molecular genetic understanding for how common dietary chemicals i.e. nutrition) influences health by altering the expression and/or structure of an individual's genetic makeup, the more restricted view is governed by the same principles as seen with advent of pharmacogenomics in clinical medicine which involves DNA based—targeted response to biologically active compounds.

In terms of dietary intervention based in individualized nutrition such examples of a number of gene-disease association studies have shown promise of this approach as follows:
Hypertension—The amount of circulating angiotensinogen (ANG) is associated with increased blood pressure. A SNP (polymorphism) designated AA, at nucleotide position −6 of the ANG gene, is linked with the level of blood ANG protein. Individuals with the AA genotype who eat the Dietary Approaches To Stop Hypertension (DASH) diet show reduced blood pressure, but this diet was less effective for carriers of the GG genotype.
Cardiovascular Apo-A1 gene plays a role in lipid metabolism and coronary heart disease. The

A allele (variant) was associated with decreased serum HDL levels. The variant was coupled with consumption of type of fat and subsequent effect on HDL levels in both males and females carrying different genotypes.
Cancer—Methylene Tetrahydrofolate Reductase (MTHFR) is a key gene in one-carbon metabolism and, indirectly, in all methylation reactions. The C677T polymorphism of this gene, which reduces enzymatic activity, is inversely associated with occurrence of colorectal cancer and acute lymphocyte leukemia. Low intake of folate, B12, B6 and methionine was associated with increased for cancer among those with the MTHFR TT genotype.
Rheumatoid arthritis—Polymorphisms in the proinflammatory cytokine tumor necrosis factor (TNF) impart a differential response to fish oil supplementation to treat rheumatoid arthritis.
Oxidant stress and inflammation—Polymorphisms in the TNF gene impart a differential response to vitamin E to promote anti-oxidant activity and reduce inflammatory processes.
Carbohydrate metabolism-Based on polymorphisms in the gene called carbohydrate responsive element-binding protein (ChREBP), a key regulator of glucose metabolism and fat storage, Cyclic AMP and a high fat diet inhibit ChREBP and slow down glucose utilization.
Obesity—In overweight women carriers of the C polymorphisms of the Leptin receptor gene lost more weight in response to low calorie diet than the non carriers.
Central Nervous System—Extracts of Ginkgo biloba induce differential expressions of 43 cortex genes, 13 hippocampus genes, and four other genes common to both brain regions.

A Case Study: Chromium and Dopamine Genes. The inventors embarked on a study with chromium picolinate to test out the principles of nutrigenomics. In this study they genotyped obese subjects for the dopamine D2 receptors gene (DRD2). The subjects were assessed for scale weight and for percent body fat. The subjects were divided into matched placebo and chromium picolinate (CrP) groups. The sample was separated into two independent groups; those with either an A1/A1 or A1/A2 allele and those with only the A2/A2 allelic pattern The measures of the change in fat weight, change in body weight, the percent change in weight, and the body weight change in kilograms were all significant, whereas no significance was found for any parameter for those subjects possessing a DRD2 A1 allele. These results suggest that the dopaminergic system, specifically the density of the D2 receptors, confers a significant differential therapeutic effect of CrP in terms of weight loss and change in body fat. Moreover, the inventors propose for the first time that mixed effects now observed with CrP administration in terms of body composition, may be resolved by typing the patient via DRD2 genotyping prior to treatment with chromium salts.

In terms of obesity research it is noteworthy that genetic manipulation in nutrition metabolism may involve current standard methods for overexpressing, inactivating, or manipulating genes. These molecular biology procedures can be carried out with the maintenance of the genetic information to subsequent generations (transgenic technology) or devised to exclusively transfer the genetic material to a given target organism, which cannot be transmitted to the future progeny (gene therapy). Moreover, the novel technique of RNA interference (RNAi) approach allows for the creation of new experimental models by transient ablation of gene expression by degrading specific mRNA, which can be applied to assess different biological functions and mechanisms.

LifeGen intends on pursuing additional DNA tests, algorithms, and nutraceutical formulations as product lines and indications related all common healthcare concerns, including but not limited to:

Alcoholism affecting 12,264,000 American

Drug Addiction affecting 12,500,000 Americans

Smoking Addiction affecting 46,000,000 Americans

Obesity affecting 60,000,000 Americans

Attention Deficit Hyperactivity Disorder affecting 11,200,000

Pre-Menstrual Dysphorric Disorder affecting 4,000,000 Americans

Pain sensitivity intolerance

Gene—nutrition interactions especially related to genome based response will indeed be the next cornerstone of solid scientific approaches to assist individuals in choosing dietary supplements, functional foods, and even nutritional beverages on an individualized basis. Nutrigenomics is the key to what we have termed “nutritional gene therapy” and from its origin will spring gene mapping as the wave of the future in nutrition.

Reward Deficiency Syndrome

Reward Deficiency Syndrome (RDS)—In order to understand the potential role of RDS as a link to inflammation, pain, and other conditions, we provide important information as a way of background in support of the novel formulae so proposed in this application. Since dopamine is a major component in mechanisms involving RDS and brain function and certain polymorphisms of the dopamine D3 receptor gene plays a role in the function of prostaglandin induced transcription activity, RDS seems to be linked. The Reward Deficiency Syndrome (RDS) results from a dysfunction in the Brain Reward Cascade which directly links abnormal craving behavior with a defect in the DRD2 Dopamine Receptor Gene as well as other dopaminergic genes (D1, D3, D4, and D5). Dopamine is a very powerful neurotransmitter in the brain, which controls feelings of well being. This sense of well-being is produced through the interaction of dopamine and neurotransmitters such as serotonin, the opioids, and other powerful brain chemicals. Low serotonin levels are associated with depression. High levels of the opioids (the brain's opium) are associated with a sense of well-being. Kenneth Blum has termed the complex interactions of these powerful neurotransmitters ultimately regulating the Dopaminergic Activity in the Reward Center of the Brain as “The Brain Reward Cascade”.

Reward Deficiency Syndrome involves a form of sensory deprivation of the brain's reward or pleasure mechanisms. Reward Deficiency Syndrome can be manifested in relatively mild or severe forms that follow as a consequence of an individual's biochemical inability to derive reward from ordinary, everyday activities. We believe that we have discovered at least one genetic aberration that leads to an alteration in the reward pathways of the brain. It is a variant form of the gene for the dopamine D2 receptor, called the A1 allele. This genetic variant also is associated with a spectrum of impulsive, compulsive, and addictive behaviors. The concept of the Reward Deficiency Syndrome unites those disorders and may explain how simple genetic anomalies give rise to complex aberrant behavior.

This patent application will highlight the importance of a new concept, which provides a clearer understanding of impulsive, addictive, and compulsive behaviors. It is our notion that the real genesis of all behavior, whether so-called normal (socially acceptable) or abnormal (socially unacceptable) behavior, derives from an individual's genetic makeup at birth. This predisposition, due to multiple gene combinations and polymorphisms, is expressed differently based on numerous environmental elements including family, friends, educational status, economical position, environmental pollutants, and availability of psychoactive drugs including food. We believe the core of predisposition to these behaviors is a set of genes which promote a feeling of well-being via neurotransmitter interaction at the “reward site” of the brain (located in the meso-limbic system), leading to normal dopamine release. We also subscribe to the notion that at least one major gene, the dopamine D2 receptor gene, is responsible for the synthesis of dopamine D2 receptors. And further depending on the genotype (allelic form A1 versus A2), the dopamine D2 receptor gene dictates the number of these receptors at post-junctional sites.

In the past nine years scientists have pursued the association between certain genes and various behavioral disorders. The list is long and remarkable—it comprises overeating and obesity, Tourette Syndrome, attention deficit and hyperactivity disorder (as well as just ADD) and pathological gambling. We believe these disorders are linked by a common biological substrate, a “hard-wired” system in the brain (consisting of cells and signaling molecules) that provides pleasure in the process of rewarding certain behavior. Consider how people respond positively to safety, warmth and a full stomach. If these needs are threatened or are not being met, we experience discomfort and anxiety. An inborn chemical imbalance that alters the intercellular signaling in the brain's reward process could supplant an individual's feeling of well-being with anxiety, anger or a craving for a substance that can alleviate the negative emotions. This chemical imbalance manifests itself as one or more behavioral disorders termed “Reward Deficiency Syndrome.”

This syndrome involves a form of sensory deprivation of the brain's pleasure mechanisms. It can be manifested in relatively mild or severe forms that follow as a consequence of an individual's biochemical inability to derive reward from ordinary, everyday activities. The inventors believe that we have discovered at least one genetic aberration that leads to an alteration in the reward pathways of the brain. It is a variant form of the gene for the dopamine D2 receptor, called the A1 allele (low D2 receptors), which may have been the natural prehistoric trait. This is the same genetic variant that was previously found to be associated with alcoholism as well as obesity (see below).

We look at evidence suggesting the A1 allele also is associated with a spectrum of impulsive, compulsive, and addictive behaviors, including a predisposition to overeating. The concept of the Reward Deficiency Syndrome unites these behaviors (impulsive/addictive/compulsive) and may explain how simple genetic anomalies give rise to complex aberrant behavior. Oddly enough, compared to the so called “normal” variant the A2, which occurs in approximately two-thirds of Americans having a normal compliment of D2 receptors, the A1 carriers may be predisposed to overeating, have a higher percent body fat, and have innate craving for carbohydrates.

The binding of the neurotransmitter to a receptor on a neuron, like a key in a lock, triggers a reaction that is part of the cascade. Disruption of these intercellular cascades results in one form or another of the Reward Deficiency Syndrome.

The Cascade Theory of Reward—The research on the neuropharmacological basis of dependence on alcohol, opiates, cocaine and glucose points to the involvement of common biochemical mechanisms. It appears as if a limbic-accumbens-pallidal circuit is the critical substrate for the expression of drug reward. However, while each substance of abuse appears to act on this circuit at a different step, the end result is the same, the release of dopamine the primary chemical messenger of reward at such reinforcement sites as the NAcc and the hippocampus. In a normal person, neurotransmitters (the messengers of the brain) work together in a pattern of stimulation or inhibition, the effects spreading downward from complex stimuli to complex patterns of response like a cascade, leading to feelings of well-being: the ultimate reward (Cascade Theory of Reward). Although the neurotransmitter system is too complex and still not completely understood, the main central reward areas in the human brain's meso-limbic system are summarized in Drawings 3a &3b.

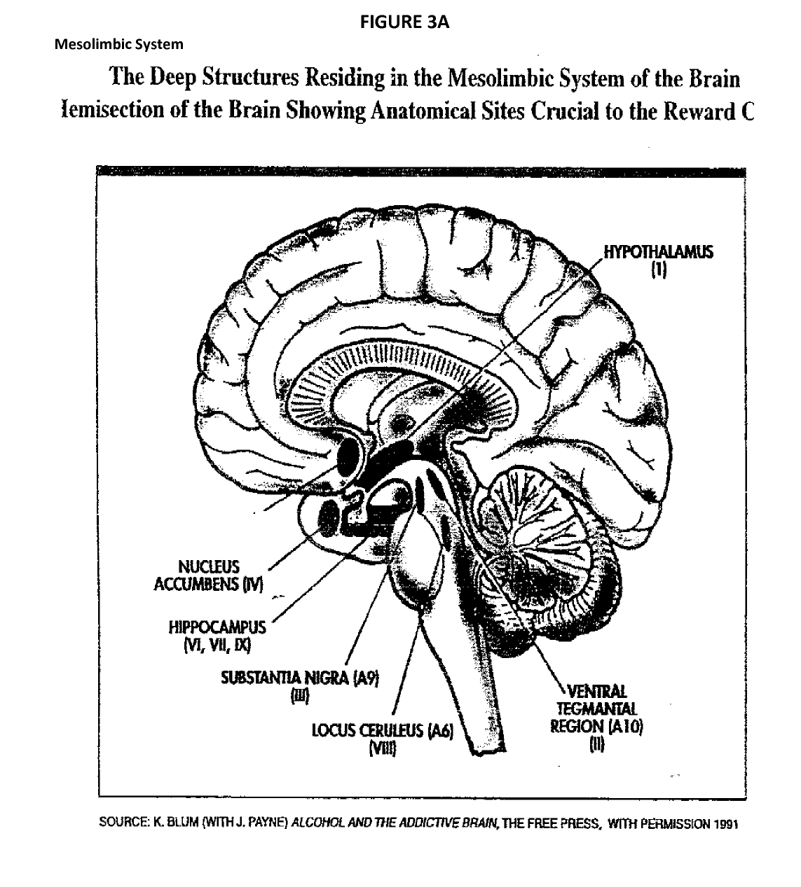

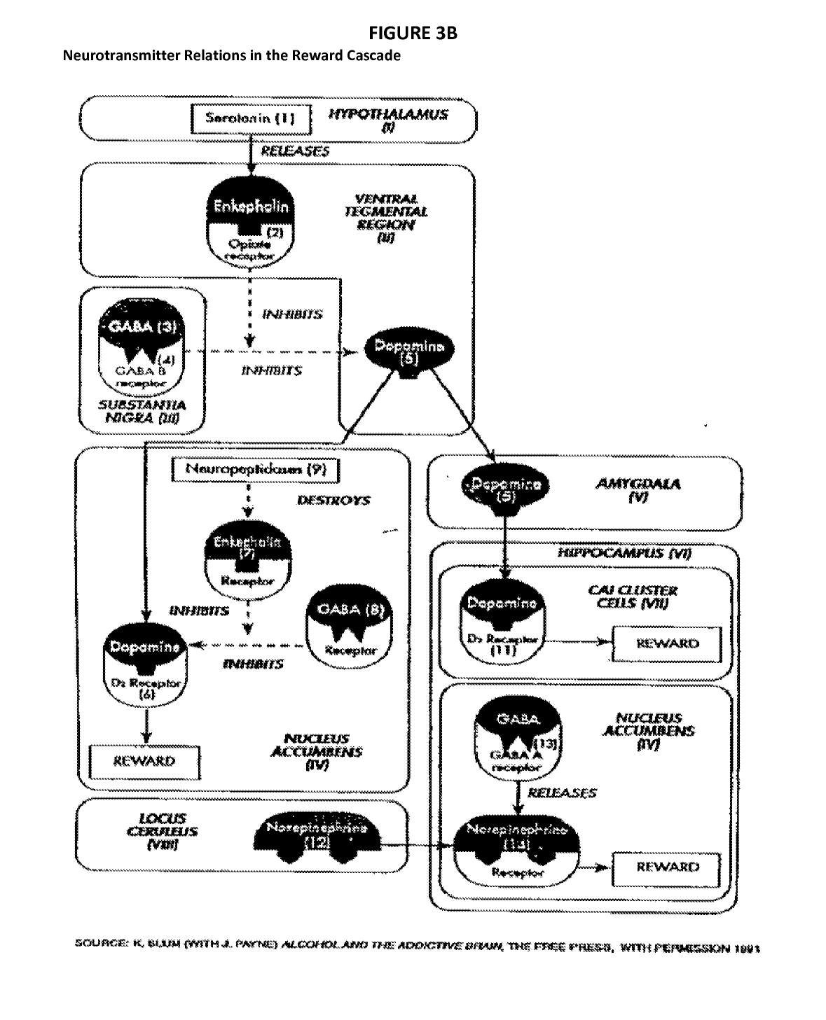

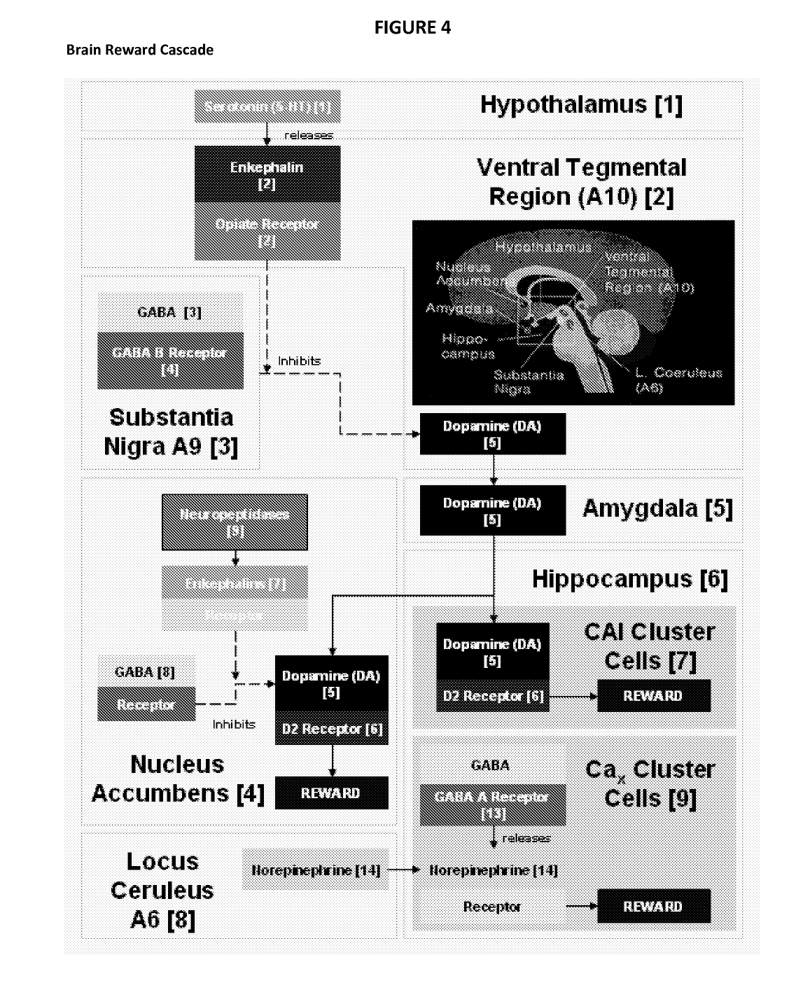

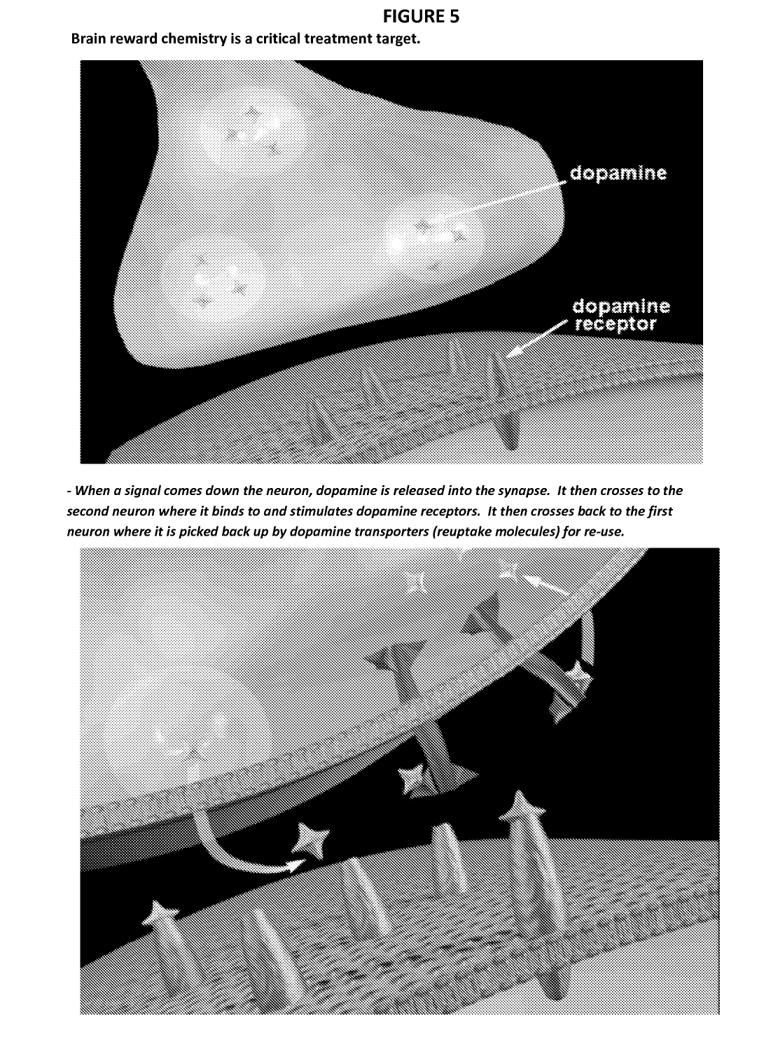

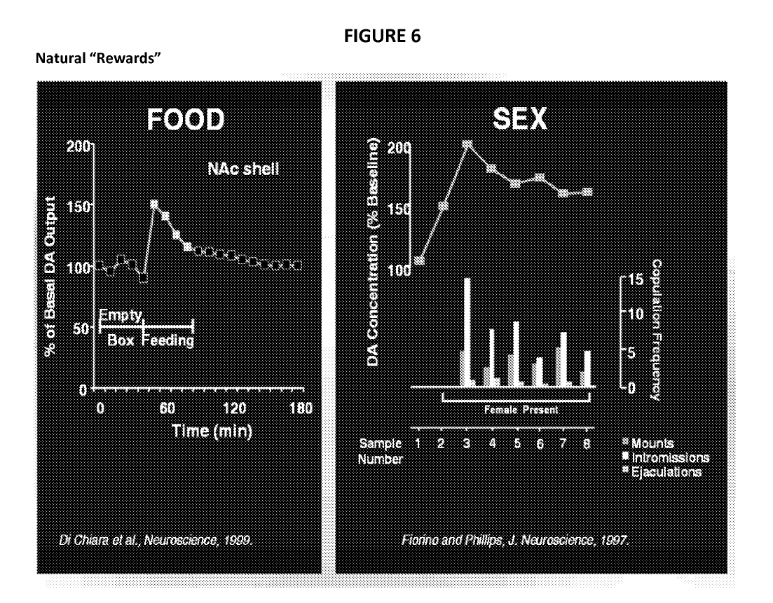

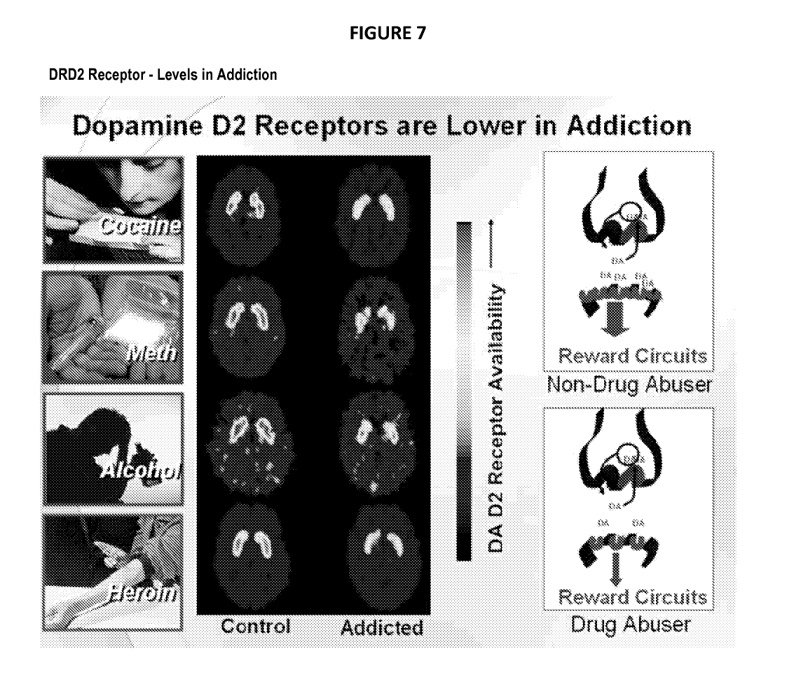

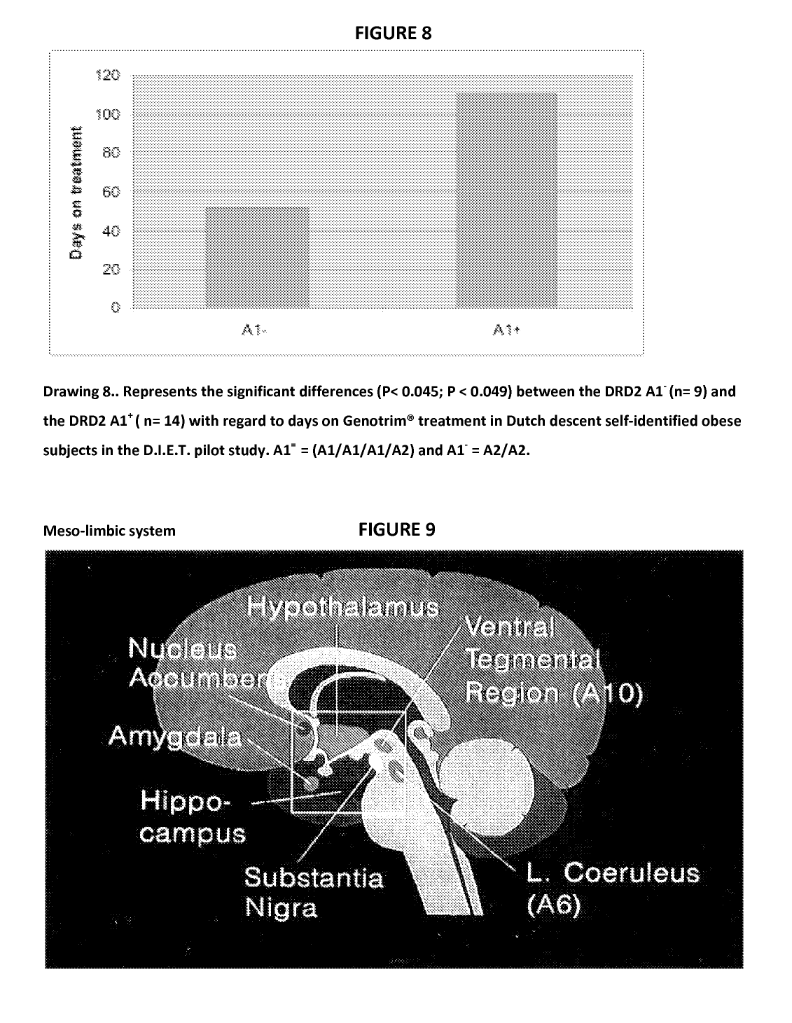

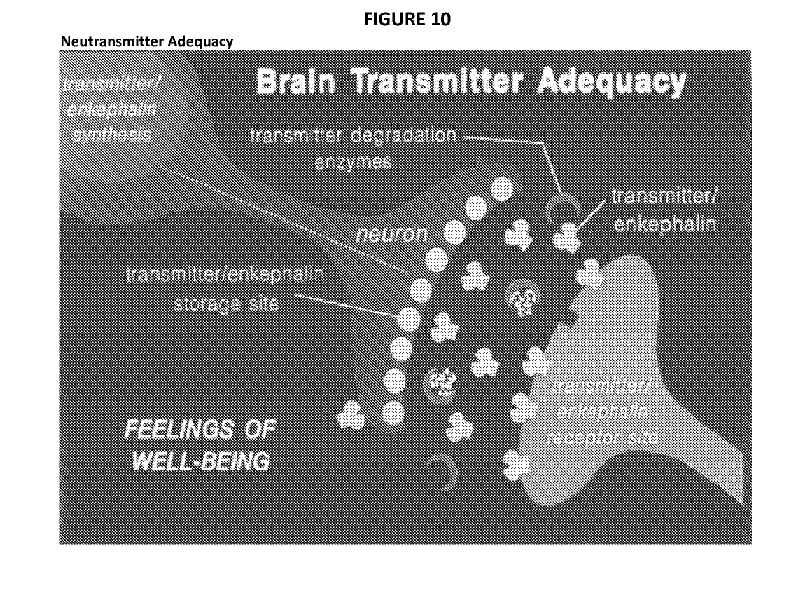

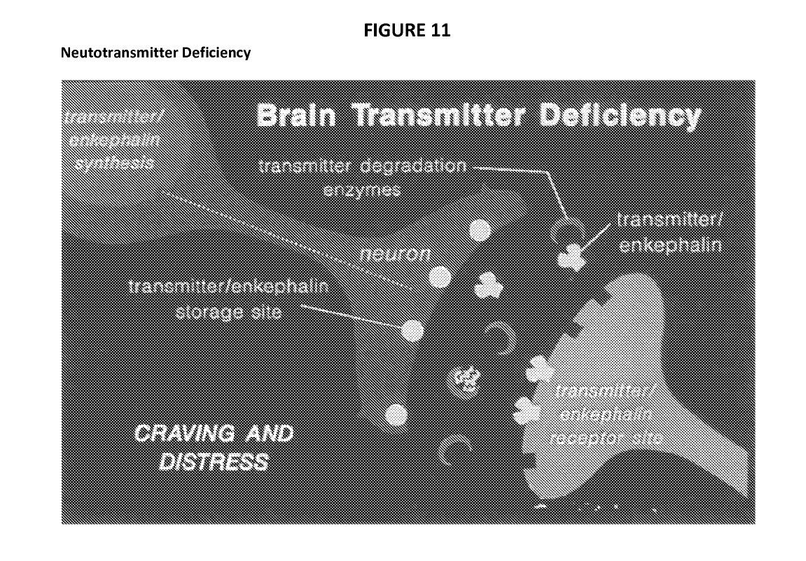

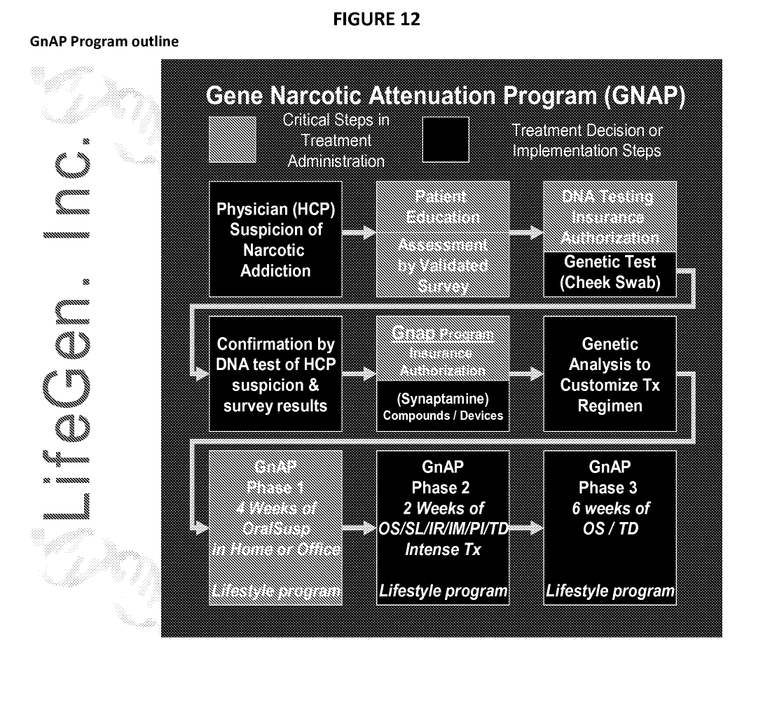

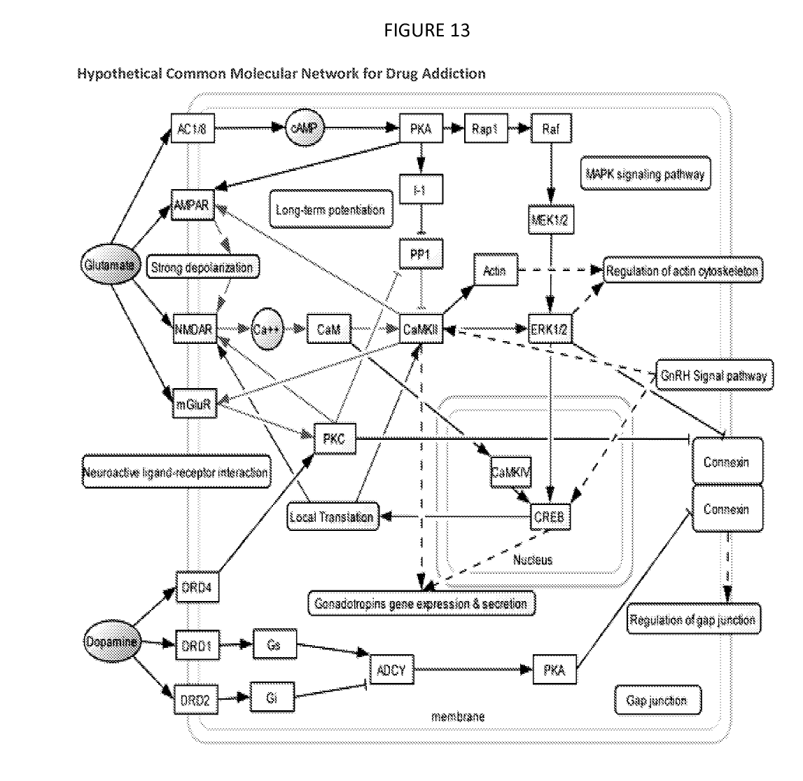

In the reward areas the following interactions take place:
serotonin (1) in the hypothalamus (I) indirectly activates opiate receptors (2) and causes a release of enkephalins in the ventral tegmental region A10 (II). The enkephalins inhibit the firing of GABA (3), which originates in the substantia nigra A9 region (III);
GABA's normal role, acting through GABA B receptors (4), is to inhibit and control the amount of dopamine (5) released at the ventral tegmental regions (II) for action at the nucleus accumbens (IV). When the dopamine is released in the nucleus accumbens it activates dopamine D2 receptors (6), a key reward site [there are at least five dopamine receptors, including D2]. This release also is regulated by enkephalins (7) acting through GABA (8). The supply of enkephalins is controlled by the amount of the neuropeptidases (9), which destroy them.
dopamine also may be released into the amygdala (V). From the amygdala, dopamine (10) reaches the hippocampus (IV) and the CA, cluster cells (VII) stimulates dopamine D2 receptors (11), another reward site.
an alternate pathway involves norepinephrine (12) in the locus of ceruleus A6 (VIII) whose fibers project into the hippocampus at a reward area centering around cluster cells which have not been precisely identified, but which have been designed a CAx (IX). When GABA A receptors (13) in the hippocampus are stimulated, they cause the release of norepinephrine (14) at the CAx site (See FIG. 3b).

It is to be noted that the glucose receptor (GR) in the hypothalamus is intricately involved and “links” the serotonergic system with opioid peptides leading to the ultimate release of dopamine at the n. accumbens. In the “cascade theory of reward” as defined by Blum and Kozlowski, these interactions may be viewed as activities of subsystems of a larger system, taking place simultaneously or in sequence, merging in cascade fashion toward anxiety, anger, low self-esteem, or other “bad feelings” or toward craving for a substance that will make these bad feelings go away, for example sugar. Certainly, many overweight individuals also cross abuse other psychoactive substances (e.g. alcohol, cocaine, and nicotine). Alcohol activates the norepinephrine fibers of the mesolimbic circuitry through a cascade of events, including the interaction of serotonin, opioid peptides, and dopamine. In a more direct fashion, through the subsequent formation of the neuroamine condensation products TIQs, alcohol may either interact with opioid receptors or directly with dopaminergic systems.

In the cascade theory of carbohydrate bingeing, genetic anomalies, long-continued stress, or long-term abuse of sugar can lead to a self-sustaining pattern of abnormal craving behavior in both animals and humans. Animal model support for the cascade theory can be derived from a series of experiments carried out by T. K. Li et al. upon their substance-preferring (P) [seek carbohydrates, alcohol, opiates, etc.] and nonpreferring (NP) rat lines. They found that P rats have the following neurochemical profile:

lower serotonin neurons in the hypothalamus;

higher levels of enkephalin in the hypothalamus (due to a lower release);

more GABA neurons in the nucleus accumbens;

reduced dopamine supply at the nucleus accumbens;

reduced densities of dopamine D2 receptors in the meso-limbic areas.

This suggests a four-part cascade sequence leading to a reduction of net dopamine release in a key reward area. This was further confirmed when McBride et al. found that administering substances which increase the serotonin supply at the synapse, or by stimulating dopamine D2 receptors directly, craving behavior could be reduced. Specifically, D2 receptor agonists reduce alcohol intake in high alcohol preferring rats whereas D2 dopamine receptor antagonists increase alcohol drinking in these inbred animals.

Inhibitors of Enkephalinase(s) and Craving Behavior—As stated earlier, although it is known that opiates and/or opioids reportedly increase food intake in animals and humans, some papers suggest the opposite-suppression of food intake, especially when one considers macro selection of food sources (i.e., sugar/carbohydrates). Moreover, Broekkamp et al. reported that infusion of enkephalin into the ventral tegmental A10 area of the brain induces a short-term latency behavioral stimulant effect reminiscent of effects produced by stimulation of the meso-limbic dopamine pathway; this effect is blocked by pretreatment of the opiate receptor antagonist naloxone. This takes on importance in terms of feeding behavior, as feeding has been shown to increase dopamine levels in various brain structures such as the posterior hypothalamus, the nucleus accumbens, and the amygdala.

It is well known that dopamine in sufficient concentration can inhibit food intake. Gilman and Lichtingfeld proposed as an appropriate therapeutic for carbohydrate bingeing (i.e., bulimia) a selective D2 agonist such as bromocriptine [or natural released dopamine], providing D2 occupancy. In this regard, using a push-pull cannula technique, Chesselet et al. were able to induce dopamine release in the “brain reward center” after local application of enkephalin, which suggests regulation by delta receptor stimulation. Indeed Kelotorphan (an inhibitor of the opioid peptide degrading enzyme) may protect against possible cholecystokinin-8 (CCK-8) degradation by brain peptidases. This important satiety neuropeptide is co-localized with dopamine in the nucleus accumbens, and there is a close interaction between CCK-8, dopamine, and endogenous opioid peptides (like enkephalins). The opioid peptides are involved not only in macro-nutrient intake, but have been implicated in substance seeking, as well as brain self-stimulation behavior. In essence, there are a substantial number of animal experiments which support not only the “Brain Reward Cascade” but the subsequent sequela induced by a defected reward cascade leading to a number of addictive, compulsive and impulsive behaviors-defined as the “Reward Deficiency Syndrome”.

In this regard, Blum et al. reversed alcohol-seeking behavior in genetically preferring C57B1/6J mice with the chronic administration of an enkephalinase inhibitor. In other work by George et al., they concluded that a relative lack of enkephalin peptides trans-synaptically, possibly resulting from enhanced enkephalin degradation, might contribute to increased alcohol consumption in C57B1/6J mice. Moreover, others showed that intracranial self-stimulation by rats was reduced by nucleus accumbens microinjections of kelatrophan, a potent enkephalinase inhibitor.

Brain Hypodopaminergic Function and The Self-Healing Process—Scientists believe individuals self-heal through biochemical (licit or non-illicit) attempts to alleviate the low dopaminergic brain activity via drug-receptor activation (alcohol, heroin, cocaine, and glucose). It is conjectured this will substitute for the lack of reward and yield a temporary sense of well-being.

Reward Deficiency Syndrome: Human Studies—Human support for the Reward Deficiency Syndrome can be derived from a series of clinical trials with neuronutrients (precursor amino acid loading technique and enkephalinase inhibition) indicating:

Reduced alcohol and cocaine craving

Reduced stress rates

Reduction of leaving treatment against medical advice (AMA)

Facilitated recovery

Reduced relapse rates

Reduction in carbohydrate bingeing

Loss of body weight

Prevention of weight regain

Reduction of glucose craving

Enhancement of insulin sensitivity

Reduction of cholesterol

Enhancement of memory and focus

Enhanced compliance with narcotic antagonists.

There are a number of studies using precursor amino-acids and enkephalinase inhibition which have been shown to affect various aspects of RDS [see below]).

Summary of Completed Clinical Studies with Nutraceutical Supplementation (A Literature Review)

| Drug | | | No. | | | |
|---|---|---|---|---|---|---|
| Abused or | Supplement | No. of | of | Study | | |
| Dysfunction | Used | Patients | Days | Type | Significant Results | Publication |
| Alcohol | SAAVE | 22 | 28 | TO | 100% decrease in | Blum K, |
| | | | | IP | BUD scores. | Trachtenberg MC, |
| | | | | | Detoxification | Ramsey J. |
| | | | | | measures: | Improvement of |
| | | | | | reduction in | inpatient |
| | | | | | benzodiazepine | treatment of the |
| | | | | | requirement, | alcoholic as a |
| | | | | | reduction in | function of |
| | | | | | withdrawal | neuronutrient |
| | | | | | tremors after 72 | restoration: a |
| | | | | | hours, reduction | pilot study. Int J |
| | | | | | in depression | Addiction. 1988; |
| | | | | | | 23: 991-98. |
| | | | | | This paper is a | Blum K, |
| | | | | | review article. | Trachtenberg MC. |
| | | | | | | Neurogenic |
| | | | | | | deficits caused by |
| | | | | | | alcoholism: |
| | | | | | | restoration by |
| | | | | | | SAAVE. Journal of |
| | | | | | | Psychoactive |
| | | | | | | Drugs. 1988; |
| | | | | | | 20: 297. |
| Alcohol | SAAVE | 62 | 21 | DBPC | Reduction in | Blum et al. |
| plus | | | | IP | psychosocial | Enkephalinase |
| Polydrugs | | | | | stress reduction | inhibition and |
| | | | | | as measured by | precursor amino |
| | | | | | SCL, reduced BESS | acid loading |
| | | | | | score, improved | improves |
| | | | | | physical score, | inpatient |
| | | | | | six-fold decrease | treatment of |
| | | | | | in likelihood of | alcoholics and |
| | | | | | leaving AMA after | poly-drug abusers: |
| | | | | | five days. | a double-blind |
| | | | | | | placebo- |
| | | | | | | controlled study |
| | | | | | | of the |
| | | | | | | neuronutrient |
| | | | | | | intervention |
| | | | | | | adjunct SAAVE. |
| | | | | | | Alcohol. 1989; |
| | | | | | | 5: 481. |
| Cocaine | Tropamine | 54 | 30 | TO | Drug hunger | Blum et al. |
| | | | | IP | significantly | Reduction of both |
| | | | | | reduced in | drug hunger and |
| | | | | | patients taking | withdrawal |
| | | | | | SAAVE as | against advice |
| | | | | | compared to | rate of cocaine |
| | | | | | controls; 4.2 | abusers in a 30 |
| | | | | | percent AMA rate | day inpatient |
| | | | | | for patients on | treatment |
| | | | | | Tropamine versus | program with the |
| | | | | | 28 percent for | neuronutrient |
| | | | | | patients on | tropamine. Curr |
| | | | | | SAAVE and 37 | Ther Res. 1988; |
| | | | | | percent for | 43: 1204. |
| | | | | | controls. | |
| Alcohol and | SAAVE and | 60 | 379 | TO | At end of one | Brown et al. |
| Cocaine | Tropamine | | | CP | year over 50 | Neurodynamics of |
| | | | | | percent of the | relapse |
| | | | | | alcoholic DUI | prevention: a |
| | | | | | offenders not | neuronutrient |
| | | | | | using SAAVE | approach to |
| | | | | | dropped out of | outpatient DUI |
| | | | | | the program | offenders. J. |
| | | | | | while less than 15 | Psychiatric Drugs. |
| | | | | | percent of those | 1990; 22: 173. |
| | | | | | using SAAVE | |
| | | | | | dropped out. For | |
| | | | | | the cocaine | |
| | | | | | abusers over 90 | |
| | | | | | percent of the | |
| | | | | | Non-Tropamaine | |
| | | | | | group dropped | |
| | | | | | out, but less than | |
| | | | | | 25 percent of the | |
| | | | | | patients in the | |
| | | | | | control group. | |
| Over-Eating | PCAL 103 | 27 | 90 | TO | The PCAL 103 | Blum et al. |
| | | | | OP | group lost an | Neuronutrient |
| | | | | | average of 27 | effects on weight |
| | | | | | pounds in 90 days | loss on |
| | | | | | compared with an | carbohydrate |
| | | | | | average loss of 10 | bingeing in a |
| | | | | | pounds for the | bariatric setting. |
| | | | | | control group. | Curr Ther Res. |
| | | | | | Only 18.2 percent | 1990; 48: 2a17. |
| | | | | | of the PCAL 103 | |
| | | | | | patient group | |
| | | | | | relapsed | |
| | | | | | compared to 82 | |
| | | | | | percent of the | |
| | | | | | patients in the | |
| | | | | | control group. | |
| Over-Eating | PCAL 103 | 247 | 730 | PCOT | After two years, | Blum K, Cull JG, |
| | | | | OP | craving and binge | Chen JHT, Garcia- |
| | | | | | eating were | Swan S, Holder |
| | | | | | reduced one-third | JM, Wood R, et al. |
| | | | | | in group of | Clinical relevance |
| | | | | | patients on PCAL | of PhenCal in |
| | | | | | 103, as compared | maintaining |
| | | | | | to the control | weight loss in an |
| | | | | | patients. PCAL | open-label, |
| | | | | | 103 group | controlled 2-year |
| | | | | | regained 14.7 | study. Curr Ther |
| | | | | | pounds of their | Res. 1997; 58: 745- |
| | | | | | lost weight | 63. |
| | | | | | compared with | |
| | | | | | 41.7 percent | |
| | | | | | weight regained | |
| | | | | | in control | |
| | | | | | patients. | |
| Over-Eating | Chromium | 40 | 112 | RDBPC | 21 percent | Kaats FE et al. The |
| | Picolinate | | | CP | increase | short-term |
| | (CP) and L- | | | | (p < 0.001) in | therapeutic effect |
| | Carnitine | | | | resting metabolic | of treating obesity |
| | | | | | rate (RMR), no | with a plan of |
| | | | | | change in lean | improved |
| | | | | | body mass (LBM), | nutrition and |
| | | | | | RMR:LBM | moderate caloric |
| | | | | | increased 25 | restriction. Curr |
| | | | | | percent (p < 0.001). | Ther Res. 1992; |
| | | | | | Body fat | 51: 261. |
| | | | | | decreased | |
| | | | | | approximately 1.5 | |
| | | | | | lbs./week, and | |
| | | | | | reduction in | |
| | | | | | serum cholesterol | |
| | | | | | while increasing | |
| | | | | | RMR with no loss | |
| | | | | | of LBM | |
| Over-Eating | Chromium | 32 | 180 | DBPC | After six months | Bahadori B, |
| | Picolinate | | | OP | the CrP group had | Habersack S, |
| | | | | | an increase in | Schneider H, |
| | | | | | lean body mass | Wascher TC, |
| | | | | | and avoided non- | Topiak H. |
| | | | | | fat related weight | Treatment with |
| | | | | | loss. Difference | chromium |
| | | | | | between groups | picolinate |
| | | | | | was significant at | improves lean |
| | | | | | p < 0.001. | body mass in |
| | | | | | | patients following |
| | | | | | | weight reduction. |
| | | | | | | Federation Am |
| | | | | | | Soc Exp Bio 1995. |
| Over-Eating | Chromium | 154 | 72 | RDBPC | 200 and 400 mcg | Kaats FE, Blum K, |
| | Picolinate | | | OP | of CrP brought | Fisher JA, |
| | | | | | about significant | Aldeman JA. |
| | | | | | changes in Body | Effects of |
| | | | | | Mass composition | chromium |
| | | | | | indicies when | picolinate |
| | | | | | compared with | supplementation |
| | | | | | placebo | on body mass |
| | | | | | | composition: a |
| | | | | | | randomized, |
| | | | | | | double-blind, |
| | | | | | | placebo- |
| | | | | | | controlled study. |
| | | | | | | Curr Ther Res. |
| | | | | | | 1996; 57:747-56 |
| Over-Eating | Chromium | 122 | 90 | RDBPC | After controlling | Kaats FE, Blum K, |
| | Picolinate | | | OP | for differences in | Pullin D, Keith SC |
| | | | | | caloric | Wood R. A |
| | | | | | expenditure and | randomized |
| | | | | | caloric intake as | double-masked |
| | | | | | compared with | placebo- |
| | | | | | the placebo | controlled study |
| | | | | | group, 400 mcg | of the effects of |
| | | | | | CrP group lost | chromium |
| | | | | | significantly more | picolinate |
| | | | | | weight (p < 0.001) | supplementation |
| | | | | | and body fat | on body |
| | | | | | (p < 0.004), had a | composition: a |
| | | | | | greater reduction | replication of |
| | | | | | in body fat | previous study. |
| | | | | | (p < 0.001), | Curr Ther Res. |
| | | | | | significantly | 1998; 59: 379-88. |
| | | | | | improve body | |
| | | | | | composition | |
| | | | | | (p < 0.004). | |
| Over-Eating | Chromium | 122 | 90 | RDBPC | Measures of | Blum K, Kaats G, |
| | Picolinate | | | OP | changes in fat | Eisenbery A, |
| | | | | | weight, change in | Sherman M, Davis |
| | | | | | body weight, | K, Comings DE, |
| | | | | | percent change in | Cull JG, Chen THJ, |
| | | | | | weight, and body | Wood R, Bucci L, |
| | | | | | weight changes in | Wise JA, |
| | | | | | kgms were all | Braverman ER, |
| | | | | | significant in | and Pullin D. |
| | | | | | A2/A2 group, and | Chromium |
| | | | | | non-significant in | Picolinate Induces |
| | | | | | A1/A2 and A1/A1 | Changes in Body |
| | | | | | carriers. | Composition as a |
| | | | | | | Function of the |
| | | | | | | Taql Dopamine |
| | | | | | | D2 Receptor A1 |
| | | | | | | Alleles. Submitted |
| | | | | | | to Advances in |
| | | | | | | Therapy. |
| Over-Eating | Chromium | 43 | 63 | ROTPC | CrP | Grant KE, |
| | Picolinate | | | OP | supplementation | Chandler RM, |
| | and | | | | resulted in | Castle AL, Ivy JL. |
| | Chromium | | | | significant weight | Chromium and |
| | Picolinate | | | | gain, while | exercise training: |
| | comparison | | | | exercise training | effect on obese |
| | | | | | combined with | women. J Am |
| | | | | | CrP | Sports Med 1997; |
| | | | | | supplementation | 29(8): 992-8. |
| | | | | | resulted in | |
| | | | | | significant weight | |
| | | | | | loss and lowered | |
| | | | | | insulin response | |
| | | | | | to an oral glucose | |
| | | | | | load. Concluded | |
| | | | | | high levels of CrP | |
| | | | | | supplementation | |
| | | | | | are | |
| | | | | | contraindicated | |
| | | | | | for weight loss, in | |
| | | | | | young obese | |
| | | | | | women. | |
| | | | | | Moreover, results | |
| | | | | | suggested that | |
| | | | | | exercise | |
| | | | | | combined with | |
| | | | | | CrP | |
| | | | | | supplementation | |
| | | | | | may be more | |
| | | | | | beneficial than | |
| | | | | | exercise training | |
| | | | | | alone for | |
| | | | | | modification of | |
| | | | | | certain CAD or | |
| | | | | | NIDDM risk | |
| | | | | | factors | |
| Healthy | Tropagen | 15 | 30 | DBPC | Non-drug subjects | Defrance JJ, |
| Volunteers | | | | OP | with Tropagen | Hymel C, |
| | | | | | performed better | Trachtenberg MC |
| | | | | | on computer | et al. |
| | | | | | memory and | Enhancement of |
| | | | | | performance | attention |
| | | | | | tasks as measured | processing by |
| | | | | | with P300 wave | Kantrol in healthy |
| | | | | | evoked potential. | humans: A pilot |
| | | | | | Changes in P300 | study. Clin |
| | | | | | wave evoked | Electroencephalgr. |
| | | | | | potential result in | 1997; 28: 68-75. |
| | | | | | better focusing | |
| | | | | | ADHD patients | |

Abbreviations used: BUD—building up to drink;
AMA—withdrawal against medical advice;
OP—outpatient;
MMPI—Minnesota Multiphasic personality inventory;
DB—double-blind;
IP— inpatient;
SCL—skin conductance level;
BESS—behavioral, emotional, social, spiritual;
DBPC—double-blind placebo-controlled;
DUI—driving under the influence;
R—randomized;
TO—open trial

The brain reward cascade schematic (DRAWING 3B), became the blueprint for the search for “reward genes”. We propose that the Reward Deficiency Syndrome gives rise to a wide range of disorders that can be classified as impulsive-addictive-compulsive diseases. Impulsive diseases include attention deficit disorder and Tourette's Disorder. Addictive diseases include substance-seeking behavior involving alcohol, drugs, nicotine, and most importantly food. Compulsive diseases include pathological gambling and excessive sexual activity. In terms of personality disorders it includes conduct disorder, oppositional defiant disorder, antisocial personality disorder, schizoid/avoidant behavior, violent aggressive behaviors (See DRAWING 1).

Reward Deficiency Syndrome (RDS), first coined by Dr. Kenneth Blum in 1995 and published in 1996, links genetic polymorphisms to a common thread of dopaminergic dysfunction leading to addictive, compulsive and impulsive aberrant behavior (Blum et al. 1996b). Many natural rewards increase dopamine neurotransmission.

Drug-induced repeated disturbances in dopamine cell activity can lead to long-term and deleterious effects in the brain. These effects can be detected using brain imaging technologies. Positron emission tomography (PET), for example, is a powerful technique that can demonstrate functional changes in the brain. The images depicted in the image below using PET show that similar brain changes result from addiction to different substances, particularly in the structures containing dopamine. Dopamine D2 receptors are one of five receptors that bind dopamine in the brain. In this image below, the brains on the left are those of normal controls, while the brains on the right are from individuals addicted to cocaine, methamphetamine, alcohol, or heroin. The striatum (which contains the reward and motor circuitry) shows up as bright red and yellow in the normal controls, indicating numerous D2 receptors. Conversely, the brains of addicted individuals (on the right row) show a less intense signal, indicating lower levels of D2 receptors. This reduction likely stems from a chronic over-stimulation of the second (post-synaptic) neuron (schematically illustrated in the right hand column), a drug-induced alteration that feeds the addict's compulsion to abuse drugs.

Gene Directed Therapeutic Targets

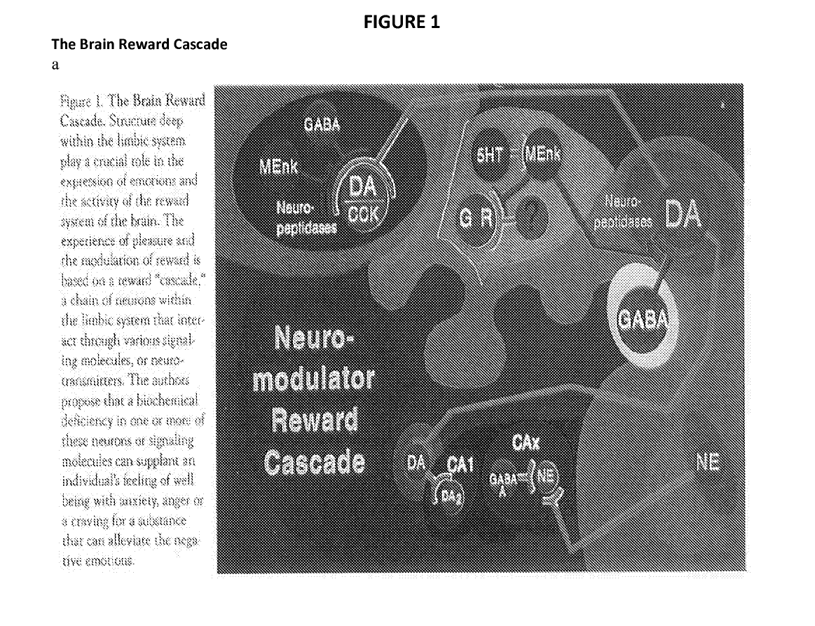

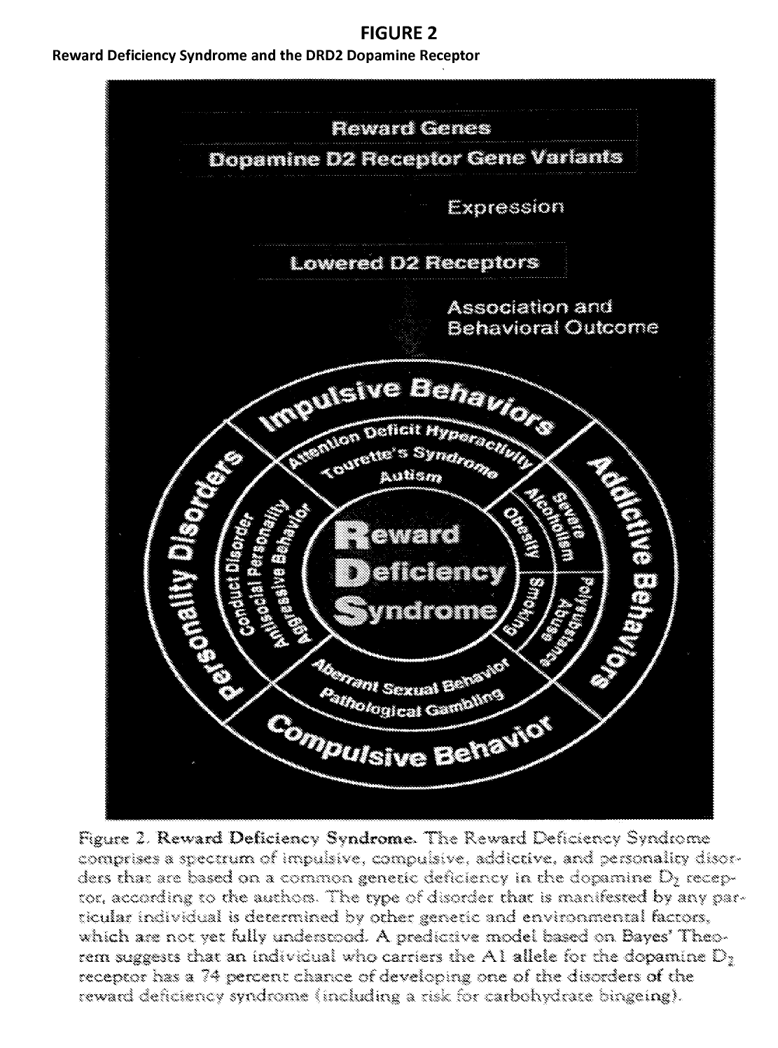

Gene therapy for many diseases seems to be the wave of the future. While we are still in its infancy some exciting research has emerged in many disciplines. Studies on rodents revealed the first successful gene therapeutic model for RDS behaviors. Nucleus accumbens injection of a viral vector carrying the cDNA (compliment DNA) of the DRD2gene resulted in an increase of D2 receptors with a concomitant reduction of alcohol seeking behavior. In terms of treatment outcomes compliance is an important issue. For most therapeutics even in the pharmaceutical field less than half of patients receiving medication actually comply. As early as 1995, it was found that certain genotypes might hold the clue to poor compliance. One example is the finding that carriers of the DRD2 A2 variant (allele) [the normal gene variant] had a higher attrition rate compared to the carriers of the DRD2 A1 variant [the RDS variant] with regard to alcoholism treatment using a DA D2 receptor activator (agonist), known as bromocriptine. Most recently this effect was confirmed in a study utilizing an experimental DNA customized nutraceutical called Genotrim. Carriers of the DRD2 A2 variant had a higher attrition rate (50.1 days on treatment), compared to the DRD2 A1 variant (110 days on treatment.). This tends to suggest that possibly the DRD2 A1 variant may be a persistency genotype that may have utility for a wide array pharmaceutical and nutraceutical modalities (see FIG. 2).

Certainly many (100's) other genes are involved. A short list includes: DRD1, DRD2, DRD3, DRD4, DRD5, DAT1, HTT, HTR1A, TD02, DBH, ADRA2A, ADRA2C, NET, MAOA, COMT, GABRA3, GABRB3, CNR1, CNRA4, NMDAR1, PENK, AR, CRF, HTR1D_HTR2A, HTR2c, interferon-_CD8A, or PS1, ANKK1, TD02, SREBP-1c, PPAR-gamma-2, MGPAT, NYP, AgRP, POMC, CART, OBR, Mc3R, Mc4R, UCP-1, GLUT4, C-FOS, C-JUN, C-MYC, Interleukin 1-alpha, interleukin-1 beta, interleukin-8, tumor necrosis factor-alpha, intracellular adhesion molecule, and interleukin-10, CYP2D6, P-glycoprotein, ABCB1, mu opioid receptor, delta opioid receptor, kappa opioid receptor, sigma opioid receptor, gamma opioid receptor, among other genes (see below).

Solution

It is our belief that if there is a genetic tendency to abuse alcohol, opiates, stimulants, carbohydrates, nicotine, especially in individuals carrying the DRD2A1 allele, which causes a one-third decrease of D2 receptors in the reward system of the brain, nutraceutical manipulation of the brain reward circuitry will be beneficial. High craving behavior may indeed be tied to low D2 receptors. Low D2 receptors are tied to DRD2A1 allele. Slow D2 agonistic action of any D2 agonist including natural dopamine, causes a slow but steady proliferation of D2 receptors even against one's genetic make up. It is also our belief that the Synaptamine Complex will cause a preferential DA relapse at the NAC which will ultimately increase D2 receptors and reduce craving behavior.

### FIELD OF INVENTION

Brain Nutrition and Behavior—A detailed account of this subject is treated in the books Alcohol and The Addictive Brain (Blum, 1991 The Free Press), and To Binge or Not to Binge? (Blum, Cull & Miller, 1998 Psychiatric Genetic Press). In short, if genetic anomalies result in neurotransmitter imbalance, then how could we help to restore balance? At the functional level, it seems clear that neurotransmitter imbalance may be a problem of brain nutrition: more specifically, a deficiency or excess of amino acids. In the healthy body, amino acids are in balance; if there is an excess or shortage, distortions of brain function can result.

As we know the brain cannot synthesize all of the amino acids involved in the formation of neurotransmitters; some are derived from food metabolism, and come to the brain via the blood supply. There are two categories of amino acids: essential and nonessential. There are five essential amino acids necessary for the manufacture of neurotransmitters, thought to play a role in obesity: methionine, leucine, phenylalanine, tyrosine, and tryptophan (see above for more detail). Among the nonessential amino acids manufactured in the body, Glutamine probably plays a significant role, because it is involved in the manufacture of GABA. Two forms of amino acids are found in nature. The amino acids in the brain that make up the neurotransmitters, and the enzymes that regulate them, are all derived from the L-form. The D-form (as in D-phenylalanine) is found in a few microorganisms and in multi-cellular organisms like frog skin.

Single Versus Multiple Amino Acid Neuronutrients

First, although a single amino acid may be involved in the formation of a given neurotransmitter, it does not act alone. It needs the help of co-factors such as vitamins and minerals before the formation can take place. For example, vitamin B6 (in the alcoholic, pyridoxal-5-phosphae form is required) is needed for the manufacture of dopamine.

Second, obesity is the result of a complex disorder that involves processes taking place in the neuron, at the synapse, and at receptors.

Third, we cannot determine (until we use DNA tests) the specific defect that is producing a particular part of the problem. Therefore, in the effort to offset neurotransmitter deficits, it is not feasible to depend on single amino acids. This is why we include both serotonergic and dopaminergic precursors.

Fourth, an odd characteristic of the blood/brain barrier actually makes treatment easier. Most overweight individuals have compounded stress and may have comorbid addictions like alcohol, smoking, and other drugs; it is known that all of these weaken the barrier facilitating the passage of restorative substances such as amino acids into the brain. This is particular important when you consider large neutral amino carrier system and competition of tryptophan, phenylalanine and tyrosine. It is equally important when you consider, as mentioned earlier, that the rate limiting enzyme Tyrosine Hydroxylase works best under stressful conditions and the precursor tyrosine will indeed be converted to dopamine and will be subsequently released into the synapse of the N. accumbens.

Fifth, it is well known that the degradation of catecholamines by COMT plays a role, albeit only partial, in clearing these neurotransmitters from synaptic cleft. Dopamine, norepinephrine and serotonin reuptake into nerve terminals via membrane transporter is thought to play a more significant role. However, it is our position that any enhancement of the neurotransmitters in the synapse is positive. In this regard, the effects of synephrine on norepinephrine receptors plus the central nervous system effects of Rhodiola rosea could contribute to a sibutramine/d-fenfluramine-like effect. The amount of Rhodiola rosea recommended in the formula is 240 mg per day (based on an extract standardized to 3% rosavin), which is somewhat higher than the recommended dose for use of Rhodiola rosea as an antidepressant (200 mg/day). Moreover, the NGI formula also contains synephrine, derived from citrus aurantium (6% synephrine) at a daily dose of 50 mg. This amounts to only 6 mg per day. While this is less than what is normally recommended as s sympathomimetic agent, when combined with caffeine thermogenesis could be achieved without the stimulatory effects seen with much higher doses (104 mg/day).

Studies Showing Anti-craving Efficacy of Precursor Amino-acids and Enkephalinase Inhibitor Activity—It is our contention that with the formula as designed for anti-craving, additive or even synergistic outcomes might be observed since the ingredients are included that could act through several different mechanisms to enhance the activity of the neurotransmitters. The patented complex has been named Synaptamine™.

In a number of experiments we have shown brain changes of the enkephalins using d-phenylalanine (500 mg/kg/day for 18 days and or its metabolite hydrocinnamic acid (intracerebral ventricular injection of 25 micrograms) in mice; Using the same doses these known enkephalinase inhibitors significantly reduced alcohol preference in both acceptance and 14 day preference test.

We have shown in healthy volunteers electrophysiological changes (enhanced memory and focus) with the combination of DL-phenylalanine (1500 mg/day), L-tyrosine (900 mg/day), L-glutamine (300 mg/day), chromium picolinate (360 micrograms/day) and other co-factors;

Positive effects in alcoholics in an in-patient hospital including lower building up to drink scores, required no PRN benzodiazepines, (0% vs. 94%), ceased tremoring at 72 hours, had no severe depression on the MMPI, in contrast to 245 of control group (Blum et al. 1988). The ingredients included Dl-phenylalanine (2760 mg/kg/day), L-tryptophan (150 mg/day), L-glutamine (150 mg/day), and pyridoxal-5-phosphate (30 mg/day);

In a double-blind placebo controlled study of polysubstance abusers in an in patient hospital, the combination of DI-phenylalanine (2760 mg/day), L-tryptophan (150 mg/day), L-glutamine (150 mg/day), and pyridoxal-5-phosphate (30 mg/day), significantly reduced stress, improved physical and emotional scores, a six-fold reduction in AMA rates, enhanced treatment recovery;

Utilizing DL-phenylalanine (1500 mg/day), L-tyrosine (900 mg/day), L-glutamine (300 mg/day), L-tryptophan (400 mg/day) and pyridoxal-phosphate (20 mg/day) in inpatient treatment of cocaine abusers over a 30 day period compared to controls significantly reduced drug hunger and withdrawal against advice rate (AMA), reduced need for benzodiazepines, and facilitated retention in the treatment program;

In an outpatient clinic DUI offenders (alcoholics and/or cocaine addicts) were treated with a combination of dl-phenylalanine, L-tyrosine, L-glutamine, Chromium, pyidoxyl-5-phosphate over a ten month period. Compared to a vitamin control (only B-complex and vitamin c), the experimental group significantly reduced relapse rates and enhanced recovery in these DUI outpatient offenders. The retention rates obtained for alcoholics was 87% for the experimental group compared to only 47% of the control patients and for cocaine abusers the numbers are 80% vs. only 13%. For alcoholics: DL-phenylalanine (2760 mg/day), L-Glutamine (150 mg/day), chromium picolinate (360 micrograms/day), pyridoxal-5-phosphate; For cocaine abusers: DL-phenylalanine (1500 mg/day), L-Tyrosine (900 mg/day), L-glutamine (300 mg/day), pyridoxal-5-phosphate (20 mg/day).

Utilizing amino-acid and enkephalinase inhibitory therapy, J.A. Cold found significant improvement in both cocaine craving and withdrawal symptoms in out patient cocaine addicts. The ingredients included DL-phenylalanine (1500 mg/day), L-Tyrosine (900 mg/day), L-glutamine (300 mg/day), pyridoxal-5-phosphare (20 mg/day).

With only chromium picolinate it was found in two double-blind placebo controlled studies that doses of either 00 mcg or 400 mcg resulted in a body composition improvement, loss of body fat, gain in nonfat mass;

In addition see above for similar results dependent on the DRD2 A1 variant (unpublished Blum & Kaats);

With DL-phenylalanine (2700 mg/day), L-tryptophan (150 mg/day), L-glutamine (150 mg/day) and pyridoxal-5 phosphate (30 mg/day) it was also found that 27 outpatients with high carbohydrate bingeing behavior where females were assigned 800 calories total intake per day and males were assigned 1,000 to 1,200 calories per day and all withdrew from sugar use attending a supervised diet-controlled treatment program, the supplement group over a 90 day period lost an average of 26.96 pounds compared to the control group (no supplement) lost only 10 pounds. In fact, only 18.2% of the experimental group relapsed (lost less than 15 pounds over the 90 day period) compared to 8. % in the control group;

In another study where the supplement contained dl-phenylalanine (2760 mg/day), L-tryptophan (150 mg/day), L-glutamine (150 mg/day), pyridoxal-5 phosphate (30 mg/day), chromium Picolinate (200 micrograms/day), and carnitine (60 mg/day) over a 2-year period in 247 obese patients the following results were obtained in a dual blind non-randomized open trial utilizing Centrum vitamin as a control: compared with the Non-PhenCal/Centrum group the experimental PhenCal/Centrum group showed a two-fold decrease in percent overweight for both males and females; a 70% decrease in food cravings for females and a 63% decrease for males; and a 66% decrease in binge eating for females and a 41% decrease for males. Most importantly, the experimental group regained only 14.7% of the lost weight, and multiple regression modeling revealed that with PhenCal treatment, morbid obesity and binge eating score were significant predictors of weight gain after 2 years. In contrast, family history of chemical dependence was most closely associated, although not statistically significant, with improved results with PhenCal.

Blum decided to test the hypothesis that possibly by combining a narcotic antagonist and amino acid therapy consisting of an enkephalinase inhibitor (D-Phenylalanine) and neurotransmitter precursors (L-amino-acids) to promote neuronal dopamine release might enhance compliance in methadone patients rapidly detoxified with the narcotic antagonist Trexan® (Duponr, 5 Del.). In this regard, Thanos et. al. and associates found increases in the dopamine D2 receptors (DRD2) via adenoviral vector delivery of the DRD2 gene into the nucleus accumbens, significantly reduced both ethanol preference (43%) and alcohol intake (64%) of ethanol preferring rats, which recovered as the DRD2, returned to baseline levels. This DRD2 overexpression similarly produced significant reductions in ethanol non-preferring rats, in both alcohol preference (16%) and alcohol intake (75%). This work further suggests that high levels of DRD2 may be protective against alcohol abuse. The DRD2 A1 allele has also been shown to associate with heroin addicts in a number of studies. In addition, other dopaminergic receptor gene polymorphisms have also associated with opioid dependence. For example, Kotler et al. showed that the 7 repeat allele of the DRD4 receptor is significantly overpresented in the opioid dependent cohort and confers a relative risk of 2.46. This has been confirmed by Li et. al. for both the 5 and 7 repeat alleles in Han Chinese case control sample of heroin addicts. Similarly Duaux et. al. in French Heroin addicts, found a significant association with homozygotes alleles of the DRD3-Bal 1. A study from NIAAA, provided evidence that strongly suggests that DRD2 is a susceptibility gene for substance abusers across multiple populations. Moreover, there are a number of studies utilizing amino-acid and enkephalinase inhibition therapy showing reduction of alcohol, opiate, cocaine and sugar craving behavior in human trials. Over the last decade, a new rapid method to detoxify either methadone or heroin addicts utilizing Trexan® sparked interest in many treatment centers throughout the United States, Canada, as well as many countries on a worldwide basis. In using the combination of Trexan® and amino-acids, results were dramatic in terms of significantly enhancing compliance to continue taking Trexan®. The average number of days of compliance calculated on 1,000 patients, without amino-acid therapy, using this rapid detoxification method is only 37 days. In contrast, the 12 subjects tested, receiving both the Trexan® and amino-acid therapy was relapse-free or reported taking the combination for an average of 262 days (P<0.0001). Thus coupling amino-acid therapy and enkephalinase inhibition while blocking the delta receptors with a pure narcotic antagonist may be quite promising as a novel method to induce rapid detox in chronic methadone patients. This may also have important ramifications in the treatment of both opiate and alcohol dependent individuals, especially as a relapse prevention tool. It may also be interesting too further test this hypothesis with the sublingual combination of the partial opiate mu receptor agonist buprenorphrine. The ingredients tested included DL-phenylalanine (2760 mg/day), L-Glutamine (150 mg/day), chromium picolinate (360 micrograms/day), pyridoxal-5-phosphate (30 mg/day).

Most recently a study was performed by Julia Ross best selling author of The Diet Cure (Viking Press USA, 1999; Penguin UK, Au, and USA, 2000), in an outpatient clinic in Mill Valley, Calif. involving amino-acid therapy and enkephalinase inhibition based on Blum's work. At Recovery Systems, Ross has successfully utilized this approach to treat a number of RDS behaviors, especially eating disorders. In a preliminary evaluation, utilizing the following

[symbol]

A study in Las Vegas at an outpatient clinic has been completed. The following results have been evaluated and presented herein. Relapse rates: CCD:—Out of 15 patients only 2 patients dropped out, while the other 13 patients remained in the program for 12 months. Therefore, the percent relapse for this group is 13.33; CC—Out of 43 patients 11 patients dropped out, while the other 32 patients remained in the program for 12 months. Therefore, the percent relapse for this group is 23.2; FCS—Out of 10 patients only 2 dropped out, while the other 8 patients remained in the program for 12 months. Therefore, the percent relapse for this group is 20.0; SR—Out of 8 patients none dropped out, thus 8 patients remained in the program for 12 months. Therefore, the percent relapse for this group is 0.0. If we calculate the percent relapse of the entire program which included a total of 76 patients with a total of 15 patients that dropped out it is a remarkable 19.9% relapse. The majority of drop outs (11 out of 15 or 73.3%) were methamphetamine abusers. the ingredients include DL-phenylalanine (2700 mg/day), 5-hydroxytryptophan (20 mg/day), L-Tyrosine (750 mg/day), L-glutamine (350 mg/day), Rhodiola rosea (3% rosavin) (66 mg/day), Chromium dinicotinate glycerate 1000 micrograms/day), DMAE (40 mg/day), Huperzine A (150 micrograms/day). Combination of vitamins (C, E, Niacin, Riboflavin, Thiamin, B6 [20% Pyridoxal-5 phosphate and 80% Pyridoxine], folic acid, B12, Biotin, Pantothenic acid, Calcium, Magnesium, zinc, Manganese and a herbal calming blend, focus blend or mood enhancing blend. The ingredients and dosage was dependent on type of abusers including diagnosis of ADHD.

Fortunately, if a broad menu of amino acids is available in sufficient quantity, the brain appears to have the ability to choose from the menu the one or ones needed to manufacture more of the neurotransmitter that is deficient. Based on the patents and technology afforded to us, the following nutrients are scientifically formulated and have been clinically tested for over 20 years and have relevance to the problem defined as “Reward Deficiency Syndrome”, more specifically-overeating and carbohydrate bingeing. However, the work to date supports a generalized anti-craving claim.
D-Phenylalanine, to inhibit enkephalinase, the enzyme that metabolizes or breakdown enkephalins, thereby increasing the availability of enkephalins and, presumably, making more dopamine available at the reward sites especially under stressful conditions.
L-Phenylalanine, to stimulate the production of dopamine, and/or increase norepinephrine levels in the reward area of the brain. The major problem with this amino acid is that it could compete with other amino acids, such as blood borne I-tryptophan and I-tyrosine at the large neutral amino-acid brain carrier system (see Milner et al. 1986). However, other data demonstrates for the first time that the synthesis and release responses to some dopaminergic agents may be elicited from synaptosomal dopamine, which is formed by the hydroxylation of phenylalanine. Amphetamine and Cogentin increased the release of dopamine formed from 14C-phenylalanine in rat caudate nucleus synaptosomal preparation and concomitantly stimulated the synthesis. Amfoelic acid also caused a net release of that dopamine. In conclusion, the results suggest that synaptosomal particles represent a unit capable of synthesizing dopamine from 1-phenylalanine and that synthesis from this precursor may be under the regulatory control of the particles.
L-glutamine, to increase brain GABA levels at receptors associated with anxiety. Its major use is to maintain balance in case of over inhibition by D-phenylalanine.
L-5-hydroxytryptophan (or its natural form)—The effect of systemic administration of 5-hydroxy-1-tryptophan on the release of serotonin in the lateral hypothalamus of the rat in vivo as examined utilizing brain microdialysis. Administration of 5-HTP caused an immediate increase of the 5-HT in dialysates, which was long lasting and dose dependent. When calcium was omitted from the perfusion medium, thereby limiting exocytosis, levels of basal 5-HT were significantly decreased and the 5-HTP-induced response of 5-HT was markedly attenuated.
Pyridoxal-5-phosphate, the active ingredient of vitamin B6 to serve as a co-factor in the production of neurotransmitters and to enhance the gastrointestinal absorption of amino acids.
Chromium Salts (Nicotinate and Picolinate), have a number of metabolic effects including: increase of insulin sensitivity; reduction of cholesterol; reduction of percent body fat; reduction of weight loss; maintaining muscle mass promoting lean; enhancing body composition; promotes brain serotonin production (see above).
Calcium, promotes neurotransmitter release based on many studies (e.g., daily administration of 5-10,000 mg Algae Cal® and/or 5-10,000 mg Coral Calcium, Sierasil)
Rhodiola rosea—Several clinical trials with double-blind placebo controls in Russia provide evidence that R. rosea possess positive mood enhancing and anti-stress properties with no detectable levels of toxicity. Generally, R. rosea extract has been shown to have a positive influence on the higher nervous system, increasing attention span, memory, strength and mobility of the human body, and weight management. It is believed that R. rosea can act as a COMT inhibitor where brain levels of serotonin and dopamine has been observed. Studies by Saratikov and Marina suggest that R. rosea can increase the level of neurotransmitters by 30 percent and decrease COMT activity by 60 percent. In the weight management area there are double-blind studies with regard to weight loss and fat mobilization
herbal component such as passion flower or fruit, Black Currant Oil; Black Currant Seed Oil; Ribes nigrum; Borage Oil; Borage Seed Oil; Borago officinalis; Bovine Cartilage; Bromelain; Ananas comosus; Cat's Claw; Uncaria tomentosa; Cetyl Myristoleate; Cetyl-M; Cis-9cetylmyristoleate; Cmo; Chondroitin Sulfate; Collagen Hydrolysate; Collagen; Gelatin; Gelatine; Gelatin Hydrolysate; Hydrolyzed [Denatured] Collagen; Devil's Claw; Devil's Claw Root; Grapple Plant; Wood Spider; Harpagophytum procumbens; Dhea-Dehydroepiandrosterone; Dmso—Dimethyl Sulfoxide; Evening Primrose Oil; Evening Primrose; Primrose; Oenothera biennis; other Oenothera species; Feverfew; Tanacetum parthenium; Fish Oil; Flaxseed; Flaxseed Oil; Flax Oil; Linseed Oil; Linum usitatissimum; Ginger; Zingiber officinale; Gingko; Gingko biloba; Ginseng; American ginseng; panax quinquefolius; Asian ginseng; panax ginseng; Siberian ginseng; eleutherococcus senticosus; GLA (Gamma-Linolenic Acid); Glucosamine; Glucosamine sulfate; glucosamine hydrochloride; N-acetyl glucosamine; Gotu Kola; Gotu Cola; Brahmi; Brahma-Buti; Indian Pennywort; Centella asiatica; Grapeseed; Grapeseed Oil; Grapeseed Extract; Vitis vinifera; Green Tea; Chinese Tea; Camellia sinensis; Guggul; Gugulipid; Guggal; Commiphora mukul; Indian Frankincense; Frankincense; Boswellia; Boswellin; Salai Guggal; Boswellia serrata; Kava Kava; Kava; Kava Pepper; Tonga; Kava Root; Piper methysticum; Melatonin; MsM (Methylsulfonylmethane); New Zealand Green-Lipped Mussel; Perna Canaliculus; Phellodendron Amurense; Sam-E (S-adenosyl-L-methione); Shark Cartilage; Cartilage; St. John's Wort; Hypercium perforatum; Stinging Nettle; Urtica dioica; Thunder God Vine; Tripterygium wilfordii; Turmeric; Curcuma longa; Curcuma domestica; Type II Undenatured Chicken Collagen; Chicken Collagen; Chicken Type II Collagen; Type II Collagen; Valerian; Valeriana officianalis; White Willow; Willow Bark; Salix Alba; White Willow Bark; Wild Yam; Discorea villosa; Ganoderma Lucidum; Mangosteen Extract; Quercetin, or combinations thereof.

Analogy—Pharmacologic Mechanisms of the Drug Meridia: Comparison Proposed Anti-Craving Formula.

Meridia is an approved FDA drug for “weight loss” and weight management. The major effect of this drug is an anti-craving action derived from its effect to inhibit the reuptake of serotonin (5HT), dopamine (DA) and norepinephrine (NE). This inhibition of neurotransmitter reuptake results in an increase in the length of time 5HT, DA, and NE are available to act in the synaptic junction, and ultimately in an amplification of the neurotransmitter effects to reduce sugar/glucose cravings.

In its simplest form, the ingredients in the patented composition proposed for anti-craving effects mirrors the Meridia mechanism and should produce similar anti-craving effects. In this section we will point out the potential of the ingredients in the proposed formula, based on a large body of neurochemical evidence concerning precursor amino-acids; the role of chromium as a tryptophan enhancing substance; d-amino acid inhibition of enkephalinase; Rhodiola as a suspected inhibitor of catechol-O-methyl transferase (COMT) as well as Synephrine, a substance that can mimic some of the effects of catecholamines. Thus it is anticipated that since the same three neurotransmitters affected by Meridia (Sibutramine), could potentially be affected by certain ingredients, it should produce similar effects. It could be hypothesized that by increasing precursor (i.e. phenylalanine, tyrosine, and chromium and or 5-hydroxytryptophane or any other neurotransmitter enhancer even via transport) intake and inhibiting enzymatic degradation by COMT greater levels of 5HT, DA would be available at the synapse. The availability of the synapse is also increased since the D-phenylalanine causes preferential release of dopamine via opioid peptide breakdown inhibition. Thus the sum total effect is very much like Meridia and the following information will assure the scientific potential of this novel natural formula.

Most recently, Balcioglu and Wurtman, measured the effects of sibutramine (Meridia), given intravenously, on brain dopamine and serotonin flux into striatal and hypothalamic dialysates of freely moving rats. While low doses of the drug had no effect, higher doses increased both serotonin and dopamine concentrations in the striatal and hypothalamic brain regions. These findings further support the neurochemical effects of sibutramine, and suggest that the drug's anti-obesity action may result from changes it produces in brain dopamine as well as serotonin metabolism. The importance here is that it provides further support for the SYNAPTAMINE formula and both serotonergic and dopaminergic anti-obesity actions.

Summary of GNAP

In essence, formulations of this type will cause the synthesis of the brain reward neurotransmitters like serotonin and catecholamines and through its effect on the natural opioids will by virtue of inhibiting GABA cause a significant release of dopamine at the nucleus accumbens. This constant release of possibly therapeutic amounts of dopamine (anti-stress substance) occupies dopamine D2 receptors, especially in carriers of the A1 allele (low D2 receptors and high glucose craving), and over time (possibly 6-8 weeks) effects RNA transcription leading to a proliferation of D2 receptors, thereby, reducing craving for aberrant substances, improving joint health and reducing the signs and symptoms of arthritis, reducing fat and optimizing, and providing anxiety relief.

### EXAMPLE

Injured Workers and High Narcotic Use

### The Problem

### Preferred Embodiment

Based on consensus of the literature and past clinical treatment programs individuals that are genetically predisposed to Substance Use Disorder (SUD) may be more prone to work related accidents. This high risk population will posses one or more gene variants (polymorphisms) related to the brain reward cascade and/or brain circuitry such as:

TABLE 1
Genetic Testing - Brain Reward Cascade Allele Genes

| | | |
|---|---|---|
| Dopaminergic | DRD2 receptor genes | Pleasure |
| Serotonergic | 5-HTT2 receptor genes | Depression |
| Endorphinergic | Pre-Enkephalin genes | Pain |
| Gabaergic | GABA_{A} receptor genes | Anxiety |
| NT Metabolizing genes | MAO and COMT genes | Enzymatic |
| | | Breakdown |
| Opiate receptor(s) | Delta, Mu, Kappa, Sigma | Pain |

Moreover, narcotic addiction must be avoided with these individuals in order to improve their eventual outcome. These workers typically are the revolving door patients one sees in case management. The cycle of (injury≈doctor visit≈narcotic Rx≈injury≈etc.) must be stopped and substituted with a healthier and more successful methodology of therapy.

2. Background:

Treatment of chronic, nonmalignant pain syndromes is to eliminate or significantly reduce the actual physical pain condition without addicting pharmaceuticals and to identify, treat and follow-up on those individuals who seem to constantly re-injure themselves.

Over 25% of the US population has some form of this genetic deficiency; it is estimated in the Workers Compensation industry that number rises to around 40%. Important to note, is that just because you have a genetic predisposition for an addictive behavior does not mean you will be an addict. Environmental triggers may expose these individuals to addiction. Some of these environmental triggers or influences are more important to some groups over others. The equation below is a prime example of the Nature vs. Nurture dilemma.

### Type I: Born Addiction—Genetic

### DCB=G_{DNT}+E

### DCB=Drug Craving Behavior

### G_{DNT}=Genetically Decreased Neurotransmitters

E=Environmental influences

Type 1 individuals have a genetic deficiency in the dopaminergic system. Environmental issues may trigger this behavior but the genetic genotype is much stronger than the environmental influence. This group of individuals will relapse very easy and are usually accident-prone. This may explain why in the workers compensation system this group represents about 35-40% of the W/C injuries. The most successful treatment for this group is a medical adjunctive dopaminergic therapy; The Gnap Program. Psychosocial counseling has a minor influence. When this group is treated correctly, this group has the greatest chance of recovery.

### Type II: Stress Addiction

### DCB=G_{NNT}+E_{S DNT}

### DCB=Drug Craving Behavior

### Gnnt=Genetically Normal Neurotransmitters

### E_{S DNT}=Environmental (Stress) Decreased Neurotransmitters

Type II individuals have no genetic deficiency and are drawn into the addiction cycle due to environmental stressful or pain conditions. A good example of this individual would be a woman who was abused as a child. Opiates and alcohol produce a euphoric condition, which will reduce stress. The most successful treatment for this group is a combination therapy of a modified Gnap program to attenuate the use of narcotics and psychosocial therapy. Psychosocial behavioral therapy is the primary treatment regime for these Type II individuals in order to reduce and or remove any negative Environmental stress influences.

### Type III: Drug Toxicity

### ACB=G_{NNT}+E_{A DNT}

### DCB=Drug Craving Behavior

### Gnnt=Genetically Normal Neurotransmitters

### E_{A DNT}=Environmental (Abuse) Decreased Neurotransmitters

Type III individuals have no genetic deficiency and are drawn into the addiction cycle due to a long-term drug abuse history of getting high. These individuals usually started taking drugs or alcohol as a social activity and have continued well into their adult life. These individuals are very difficult to treat. They need both medical adjunctive dopaminergic therapy and prolonged psychosocial counseling. Even when this group is treated correctly, they have the lowest success rate of recovery. Luckily, there are a lower percentage of these individuals in the Workers Compensation System vs. the Criminal Justice System.

The purpose of the Gnap program is to identify and correctly treat with gene therapy those individuals who are Type I. Genetic Identification is the KEY to success to isolate and successfully treat these individuals who are Type I. These individuals are the category which will run up the financial costs faster than any of the other groups. With the addition of DNA testing, we now have the tools that will allow the physician to make clinical decisions in the formulation of treatment protocols that are specific to the individual. This program is not a “one-size-fits-all” approach. We customize their specific treatment regime to their genetic footprint. This is what is meant by the statement “gene-therapy.” One of the cost effective components of the program is that we are able to treat and contain the individual with their primary treating physician or that of a specialist, there is no reason to advance this person to another level of care and cost, Detox, Rehab and Psychiatric care.

The Process

We propose that a threefold approach is needed for the successful treatment of these individuals.

The first step is very important; it is the identification of these predisposed individuals to narcotic abuse through DNA analysis. By taking a swabbing sample inside of the individual's cheek we have enough cells to perform a DNA analysis, no blood draw is required. With this information we are able utilize empirical medical evidence to categorize these individuals into the most appropriate treatment group. The current mode of differential diagnoses is to give your best educated guess as to which group they belong to and use a trial by error methodology in order to find the most effective course of treatment. Just this one step alone will save hundreds of thousands of dollars by utilizing gene-therapy during the early stages of treatment instead of an ineffective trial by error methodology. Unfortunately, patients are not obtaining this service at an early treatment intervention but obtaining this genetic testing later down the road of medical treatment usually at Pain Clinic's.

This condition has been treated through behavioral modification or other non-medical therapies over the past 40 years with a low success rates due to a lack of specific identification of these individuals. DNA testing is the key to the Gnap program. With the appropriate identification of these individuals, the prescribing physician can attenuate these individuals off narcotics and assist the employee to become a functional employee within an office setting environment. The cost savings for the employer is substantial. In 2005, ACOEM saw the potential cost savings industry wide and approved genetic testing within the workplace. The Gnap program adheres to all the DNA protocols established by ACOEM.

The second step is the treatment of the RDS by augmenting and balancing the pleasure chemicals in the brain called neurotransmitters (NT) without negative side effects.

Depending upon the DNA genetic results of addiction severity, the individual is placed on either a high-level or a low-level treatment regime in many administrative forms of Synaptamine™, for example in prescription compounded oral suspension or IM injections, in order to obtain the highest possible level of success.

Active treatment duration is 3 months. This program is meant to rebuild the dopamine receptor sites, giving the individual a greater sense of pleasure and well-being, essentially stopping the drug seeking and relapse behaviors. Thus, attenuating the individual from their Narcotic medication and increasing their functional status while at the same time drastically reducing costs. Another benefit of increased Dopamine is a rising of the patient's pain threshold; patients are able to cope with more of their existing pain than they were before. (See drawings 2 & 3)

The individual also has overlap of true physical pain that needs to be addressed since a non-narcotic treatment intervention is being implemented. For the third step the patient is placed on a non-addictive alternative for pain control. There are a myriad of pain devices and weak acting pain medications on the market today. These will be utilized on a trial basis to see which modality or medication is best suited for the individual. When all the components of the Gnap program are utilized opiate addicts can be drug free in three months without a Psychiatric claim or the use of a Detox/Rehab facility.

Synaptamine Formulation

TABLE 1
AMINO ACID NUTRITION THERAPY

| | | Addictive | | Expected |
|---|---|---|---|---|
| Supplemental | Restored Brain | Substance | Amino Acid Deficiency | Behavior |
| Ingredient | Chemical | Abuse | Symptoms | Change |
| D-Phenylalanine or | Enkephalins | Heroin, | Most Reward Deficiency | Reward |
| DL-Phenylalanine | Endorphins | Alcohol, | Syndrome (RDS) | stimulation. |
| | | Marijuana, | conditions sensitive to | Anti-craving. |
| | | Sweets, | physical or emotional | Mild anti- |
| | | Starches, | pain. Crave comfort and | depression. Mild |
| | | Chocolate, | pleasure. Desire certain | improved |
| | | Tobacco | food or drugs. D- | energy and |
| | | | phenyalan8ine is a | focus. D- |
| | | | known enkephalinase | Phenylalanine |
| | | | inhibitor. | promotes pain |
| | | | | relief, increases |
| | | | | pleasure. |
| L-Phenylalanine or | Norepinephrine | Caffeine, | Most Reward Deficiency | Reward |
| L-Tyrosine | Dopamine | Speed, | Syndrome (RDS) | stimulation. |
| | | Cocaine, | conditions. Depression, | Anti-craving. |
| | | Marijuana, | low energy. Lack of | Anti-depression. |
| | | Aspartame, | focus and | Increased |
| | | Chocolate, | concentration. | energy. |
| | | Alcohol, | Attention-deficit | Improved |
| | | Tobacco, | disorder. | mental focus. |
| | | Sweets, | | |
| | | Starches | | |
| L-Tryptophan or 5 | Serotonin | Sweets, | Low self-esteem. | Anti-craving. |
| hydroxytryptophan | | Alcohol, | Obsessive/compulsive | Anti-depression. |
| (5HTP) | | Starch, | behaviors. Irritability or | Anti-insomnia. |
| | | Ecstasy, | rage. Sleep problems. | Improved |
| | | Marijuana, | Afternoon or evening | appetite control. |
| | | Chocolate, | cravings. Negativity. | Improvement in |
| | | Tobacco | Heat intolerance. | all mood and |
| | | | Fibromyalgia, SAD | other serotonin |
| | | | (winter blues). | deficiency |
| | | | | symptoms. |
| GABA (Gamma- | GABA | Valium, | Feeling of being | Promotes |
| amino butyric acid) | | Alcohol, | stressed-out. Nervous. | calmness. |
| | | Marijuana, | Tense muscles. Trouble | Promotes |
| | | Tobacco, | relaxing. | relaxation. |
| | | Sweets, | | |
| | | Starches | | |
| L-Glutamine | GABA (mild | Sweets, | Stress. Mood swings. | Anti-craving, |
| | enhancement) | Starches, | Hypoglycemia. | anti-stress. |
| | Fuel source for | Alcohol | | Levels blood |
| | entire brain | | | sugar and mood. |
| | | | | GABA (mild |
| | | | | enhancement). |
| | | | | Fuel source for |
| | | | | entire brain. |

Rhodiola rosea has been added to the formula and is a known Catechol-O-methyl transferase inhibitor (COMT). This provides more synaptic dopamine in the VTA/NAc.
Source: Perfumi M, Mattioli L. Adaptogenic and central nervous system effects of single doses of 3% rosavin and 1% salidroside Rhodiola rosea L. extract in mice. Phytother Res..21 2007 37-43.
Chromium salts - This has been added to the formula to enhance insulin sensitivity and resultant brain concentrations of serotonin.
Note:
To assist in amino-acid nutritional therapy, the use of a multi-vitamin/mineral formula is recommended. Many vitamins and minerals serve as co-factors in neurotransmitter synthesis. They also serve to restore general balance, vitality and well-being to the Reward Deficiency Syndrome (RDS) patient who typically is in a state of poor nutritional health. The utilization of GABA is limited due to its polar nature and ability to cross the blood brain barrier. Glutamate is used in a low level only to prevent over-inhibition of enkephalin breakdown and subsequent inhibition of Gabaergic spiny neurons of the substantia nigra.

In terms of formulation we propose a number of forms for the delivery of Synaptamine. These include but are not limited to the following:

Oral—Pills, Capsules, tablets, Sublingual, Troche, dissolvable paper thins

Liquid—Oral suspension, beverage

Injectable—Intramuscular, Intravenous, intrathecal

Intra-Rectal

Ointments

Patches

Pellets

Beverages with powder application

Genes and Opiate Addiction: A Pharmacogenomic Trieste

In terms of pain sensitivity certain candidate genes have been studies. Candidate genes such as those for catechol-O-methyltransferase, melanocortin-1 receptor, guanosine triphosphate cyclohydrolase and mu-opioid receptor have been intensively investigated, and associations were found with sensitivity to pain as well as with analgesic requirements in states of acute and chronic pain. In contrast, the impact of genetic variants of drug-metabolizing enzymes on the response to pharmacotherapy is generally well described. Polymorphisms of the cytochrome P450 enzymes influence the analgesic efficacy of codeine, tramadol, tricyclic antidepressants and nonsteroidal anti-inflammatory drugs. Together with further candidate genes, they are major targets of ongoing research in order to identify associations between an individual's genetic profile and drug response (pharmacogenetics). Moreover, sensitivity and tolerance to morphine were determined in 2 strains of mice, BALB/cBy and C57BL/6By, their reciprocal F1 hybrids and seven of their recombinant inbred strains. Sensitivity was established based on locomotor activity following the administration of saline, 10 or 20 mg/kg of morphine hydrochloride while tolerance was established according to the “hot plate” method following the single or repeated administration of saline, 5, 10, or 20 mg/kg of morphine hydrochloride. Results indicate that both sensitivity and tolerance to morphine are genotype-dependent and their inheritance is characterized by dominance or partial dominance.

The most common treatment for opioid dependence is substitution therapy with another opioid such as methadone. The methadone dosage is individualized but highly variable, and program retention rates are low due in part to non-optimal dosing resulting in withdrawal symptoms and further heroin craving and use. Methadone is a substrate for the P-glycoprotein transporter, encoded by the ABCB1 gene, which regulates central nervous system exposure. ABCB1 genetic variability influenced daily methadone dose requirements, such that subjects carrying 2 copies of the wild-type haplotype required higher doses compared with those with 1 copy and those with no copies (98.3+/−10.4, 58.6+/−20.9, and 55.4+/−26.1 mg/d, respectively; P=0.029). In addition, carriers of the AGCTT haplotype required significantly lower doses than noncarriers (38.0+/−16.8 and 61.3+/−24.6 mg/d, respectively; P=0.04). Although ABCB1 genetic variability is not related to the development of opioid dependence, identification of variant haplotypes may, after larger prospective studies have been performed, provide clinicians with a tool for methadone dosage individualization. Studies of polymorphisms in the mu opioid receptor gene, which encodes the receptor target of some endogenous opioids, heroin, morphine, and synthetic opioids, have contributed substantially to knowledge of genetic influences on opiate and cocaine addiction. Other genes of the endogenous opioid and monoaminergic systems, particularly genes encoding dopamine beta-hydroxylase, and the dopamine, serotonin, and norepinephrine transporters have also been implicated. Moreover, genetically caused inactivity of cytochrome P450 (CYP) 2D6 renders codeine ineffective (lack of morphine formation), slightly decreases the efficacy of tramadol (lack of formation of the active O-desmethyl-tramadol) and slightly decreases the clearance of methadone. MDR1 mutations often demonstrate pharmacogenetic consequences, and since opioids are among the P-glycoprotein substrates, opioid pharmacology may be affected by MDR1 mutations. The single nucleotide polymorphism A118G of the mu opioid receptor gene has been associated with decreased potency of morphine and morphine-6-glucuronide, and with decreased analgesic effects and higher alfentanil dose demands in carriers of the mutated G118 allele. Genetic causes may also trigger or modify drug interactions, which in turn can alter the clinical response to opioid therapy. For example, by inhibiting CYP2D6, paroxetine increases the steady-state plasma concentrations of (R)-methadone in extensive but not in poor metabolizers of debrisoquine/sparteine. So far, the clinical consequences of the pharmacogenetics of opioids are limited to codeine, which should not be administered to poor metabolizers of debrisoquine/sparteine. Genetically precipitated drug interactions might render a standard opioid dose toxic and should, therefore, be taken into consideration. Mutations affecting opioid receptors and pain perception/processing are of interest for the study of opioid actions, but with modern practice of on-demand administration of opioids their utility may be limited to explaining why some patients need higher opioid doses; however, the adverse effects profile may be modified by these mutations. Nonetheless, at a limited level, pharmacogenetics can be expected to facilitate individualized opioid therapy. It has been demonstrated that the muOR 304G variant significantly reduces intrathecal fentanyl ED(50) for labor analgesia, suggesting women with the G variant may be more responsive to opioids and require less analgesic drugs. These findings for intrathecal fentanyl pharmacogenetics may have implications for patients receiving opioids in other settings. The following is a sampling of genes involved in the addictive process that we propose can be informative which relate to Opiate addiction:

mu opioid receptor, delta-opioid receptor; the metabotropic receptors mGluR6 and mGluR8, nuclear receptor NR4A2 and cryptochrome 1 (photolyase-like), DRD gene (D1-D5), Dat1, DBH, proenkephalin (PENK) and prodynorphin (PDYN), CAMKII; GnRH; CYP2D6; BDNF; NT-3 genes; GABA receptor subunit genes on 5q33; GABA(A)gamma2; OPRM1; G-protein alpha subunits; OPRK1; alpha2-adrenoceptor; TTC12; ANKK1; NCAM1; ZCRB1; CYP2B6; CYP2C19; CYP2C9; interleukin-2; RGS-R7; Gbeta5; MAO-A; 287 A/G polymorphism of catechol-O-methyltransferase; serotonin transporter; Ca2+/cAMP responsive element binding protein; CNR1; ABCB1, P-glycoprotein, UGT2B7, and CREB.

Such polymorphisms include a polymorphism in a gene encoding a Beta-adrenergic receptor; a polymorphism in a gene encoding an angiotensin converting enzyme (ACE); a polymorphism in a gene encoding an angiotensin 11 TI receptor; a polymorphism in a gene encoding cholesteryl ester transfer protein; a polymorphism in a gene encoding a potassium channel; a polymorphism in a gene encoding a cytochrome P-450 enzyme, optionally CYP2D6; a polymorphism in a gene encoding a protein product of the HER2/neu oncogene; a polymorphism of the C825T gene; a polymorphism in the APOE gene locus); a polymorphism in the CT or TT allele of the dopamine D2 receptor gene; a SNP (polymorphism) designated AA, at nucleotide position −6 of the ANG gene; a polymorphism in a gene encoding Apo-Al; a polymorphism in a gene encoding Methylene Tetrahydrofolate Reductase (MTHFR), optionally a C677T polymorphism; a polymorphism in tumor necrosis factor (TNF) gene; a polymorphism in the carbohydrate responsive element-binding protein (ChREBP) gene; a polymorphism of the Leptin receptor gene; a polymorphism of the dopamine D2 receptors gene (DRD2); a polymorphism of any of the dopamine D1, D3, D4, and D5 genes; a dopamine D2 receptor polymorphism selected from the group consisting of Ser311cys and TaqIA; a polymorphism in a c-fos gene; a polymorphism in the c-jun gene; a polymorphism in the c-myc, gene; a polymorphism in a gene encoding Sterol Regulatory Element Protein-1 (SREBP-Ic); a polymorphism in a gene encoding mitochondrial glycerol-3-phosphate acetyltransferase gene (MGPAT); a polymorphism in a gene encoding the peroxisome proliferator-activated receptor (PPAR-gamma-2) gene; the ProI2Ala polymorphism of the PPARgamma gene; a polymorphism in a gene encoding Tryptophan 2, 3-Dioxygenase (TDO2); a polymorphism in a gene encoding TCP-I; a polymorphism in a gene encoding Mc4R; a polymorphism in a gene encoding CART; a polymorphism in a gene encoding interleukin-1 beta; a polymorphism in a gene encoding tumor necrosis factor-alpha; a polymorphism in a gene encoding an intracellular adhesion molecule; a polymorphism in a gene encoding interleukin-8, a polymorphism in a gene encoding and interleukin-10; a polymorphism in a gene encoding interferon-alpha; a polymorphism in a gene encoding Ras-Protein and (HLA-DRBI 0404 and OlOlor PTPN22 R620W); the Dopamine Receptor D3 Ser9Gly (−205-G/A, −7685-G/C) polymorphism; a polymorphism in a gene encoding Glutamine:fructose-6-phosphate amidotransferase (GFPTI or GFPT 2), optionally polymorphisms in exon 14, optionally 1471V, or 3′ UTR; or a polymorphism in a gene encoding glucosamine 6-P acyltransferase; a polymorphism in Aggrecan proteoglycan allele 27; a polymorphism in a gene encoding 11-beta hydroxysteroid dehydrogenase typel; a polymorphism in a gene encoding FK506 binding protein 5; a polymorphism in a gene encoding serum/glucosteroid kinase; a polymorphism in a gene encoding tryptophan 2,3 dioxygenase; a polymorphism in a gene encoding Myelin; a polymorphism in a gene encoding a Myelin associated glycoprotein, optionally myelin oligodendrocyte glycoprotein (MOG), optionally a polymorphism in a tetranucleotide TAAA repeat (MOG4), C10991T SNP; a polymorphism in a gene encoding Edg2; a polymorphism in a gene encoding Fgfr2; a polymorphism in a gene encoding Decorin; a polymorphism in a gene encoding Brevican; a polymorphism in a gene encoding Neurotensin (NT) receptors-1; a polymorphism in a gene encoding Neurotensin (NT) receptor-2; a polymorphism in a gene encoding Neurotensin (NT) receptor-3; a polymorphism in a gene encoding Proenkephalin; a polymorphism in a gene encoding prodynorphin, optionally 946C>G; a polymorphism in a gene encoding Bdnf (Neurotrophic Factor, optionally BDNF Val66Met and −281 C>A, T allele of the C270T); a polymorphism in a gene encoding Sgk (Serum- and glucose-regulated kinase (SGK 1), optionally SNP Intron 6, Exon 8 (CC, CT, TT); a polymorphism in a gene encoding GabI; Id2; a polymorphism in a gene encoding COMT; a polymorphism in a gene encoding ANKKI; a polymorphism in a gene encoding DATI; a polymorphism in a gene encoding DBH; a polymorphism in a gene encoding HTT; a polymorphism in a gene encoding HTRIA; a polymorphism in a gene encoding HTRID; a polymorphism in a gene encoding HTR2A; a polymorphism in a gene encoding HTR2c, optionally 5-HT-2A, 5-HT 2B, 5-HT-4, and 5-HT-7); a polymorphism in a gene encoding ADRA2A; a polymorphism in a gene encoding ADRA2; a polymorphism in a gene encoding NET; a polymorphism in a gene encoding MAOA; a polymorphism in a gene encoding GABRA3; a polymorphism in a gene encoding GABRB3; a polymorphism in a gene encoding CNRI; a polymorphism in a gene encoding CNRA4; a polymorphism in a gene encoding NMDARI; a polymorphism in a gene encoding POMC; a polymorphism in a gene encoding MGPAT; a polymorphism in a gene encoding NYP; a polymorphism in a gene encoding AgRP; a polymorphism in a gene encoding OBR; a polymorphism in a gene encoding Mc3R:UCP-1; a polymorphism in a gene encoding GLUT4; a polymorphism in a gene encoding PDGS; a polymorphism in a gene encoding ALdB; a polymorphism in a gene encoding LNC2; a polymorphism in a gene encoding E23K Kir6.2; a polymorphism in a gene encoding steroid sulfatase (STS); a polymorphism G82G in PTPNI; the IVS6+G82A polymorphism; a polymorphism in a gene encoding Sulfonylurea receptor 1; a polymorphism in a gene encoding beta(3)-AR Trp64Arg; a polymorphism in a gene encoding PC1; a polymorphism in a GHRELIN gene; a polymorphism in a gene encoding FKBP5; a polymorphism in a gene encoding a VITAMIN D RECEPTOR, optionally BSMI AND FOKI; a polymorphism in a gene encoding lymphoid tyrosine phosphatase (LYP), optionally a polymorphism in a gene encoding protein tyrosine phosphatase-22 (PTPN22) gene, and a polymorphism in a gene encoding any sodium ATPAse.

Allelic analysis comprises identifying at least one mutation that is a polymorphism selected from the group consisting of a polymorphism (Rs value of SNP) of a gene encoding DRD2 (RsI800497, Rs6278, Rs6276, RsI079594, Rs6275, RsI801028, RsI076560, Rs2283265, RsIO79727, RsIO76562, RsII25394, Rs4648318, Rs4274224, Rs7131056, Rs4648317, RsI799732, RsI799978; 5HT2A(Rs6314, Rs3742278, Rs6561333, RsI923886, Rs643627, Rs2770292, RsI928040, Rs2770304, Rs594242, Rs6313; ANKKI (RS2734849, RS1800497, RsII604671, Rs4938016); OPRKI (Rs35160174, Rs35373196, Rs34709943 RS6473797) OPRMI (Rs510769, Rs553202, Rs514980, Rs561720, Rs534673, Rs524731, Rs3823010, Rs3778148, Rs7773995, Rs495491, RsI2333298, RsI461773, RsI381376, Rs3778151, Rs506247, Rs563649, Rs9479757, Rs2075572, RsI0485057, Rs540825, Rs562859, Rs548646, Rs648007, Rs9322447, Rs681243, Rs609148, Rs3798687, Rs648893); COMT (Rs737864, Rs933271, Rs5993882, Rs740603, MTRs4646312, RsI65722, Rs6269, RsI7699); SLC6A3 (RsI2516948, RsI042098, Rs40184, RsII564773, RsIII33767, Rs6876225, Rs3776512, Rs2270912, Rs6347, Rs27048, Rs37022, Rs2042449, Rs464069, Rs463379, Rs403636, Rs2617605, RsI3189021, Rs6350, Rs2975223, Rs2963238, RsII564752 Rs2975226); HTR3B(Rs3758987, Rs2276307, Rs3782025, RsI672717); NOS3 (Rs891512, RsI808593, Rs2070744, Rs3918226, Rs7830); PPARG (RsI801282, Rs2938392, RsII75542, RsI7036314, RsI805192, Rs4684847, Rs2938392, Rs709157, Rs709158, RsII75542); ChREBP (Rs3812316); FTO (Rs8050136, RsI421084, Rs9939609, RsI861868, Rs9937053, Rs9939973, Rs9940128, RsI558902, RsI0852521, RsI477196, RsI121980, Rs7193144, RsI6945088, Rs8043757, Rs3751812, Rs9923233, Rs9926289, RsI2597786, Rs7185735, Rs9931164, Rs9941349, Rs7199182, Rs9931494, RsI7817964, Rs7190492, Rs9930506, Rs9932754, Rs9922609, Rs7204609, Rs8044769, RsI2149832, Rs6499646, RsI421090, Rs2302673); TNFalpha (RsI799964, RsI800629, Rs361525, RsI800610, Rs3093662); MANEA (RsI133503); LeptinOb (Rs4728096, RsI2536535, Rs2167270, Rs2278815, RsI0244329, RsII763517, RsII760956, RsIO954173); PEMT (Rs4244593, Rs936108); MAO-A (Rs3788862, RsI465108, Rs909525, Rs2283724, RsI2843268, RsI800659, Rs6323, RsI799835, Rs3027400, Rs979606, Rs979605 RsII37070); CRH(Rs7209436, Rs4792887, RsI10402, Rs242924, Rs242941, Rs242940, Rs242939, Rs242938, RsI73365, RsI876831, RsI876828, Rs937, Rs878886 Rs242948); ADIPOQ (RsI7300539, Rs2241766); STS (RsI2861247); VDR (RsI7467825, Rs731236, RsI544410, Rs2229828, Rs2228570, Rs2238136); DBI (Rs3091405, Rs3769664, Rs3769662, Rs956309, Rs8192506); GABRA6 (Rs3811995, Rs3219151, Rs6883829, Rs3811991); GABRB3 (Rs2912582, Rs2081648, RsI426217, Rs754185, Rs890317, Rs981778, Rs2059574); MTHFR(Rs4846048, RsI801131, RsI801133, Rs2066470); MLXIPL[carbohydrate binding element] (Rs3812316, RsI7145738); VEGF (Rs2010963, Rs833068, Rs3025000, Rs3025010, Rs3025039, Rs3025053); DRD4 (Rs936460, Rs41298422, Rs3758653, Rs936461, RsI2720373, Rs747302, RsI800955, Rs916455, Rs916457, Rs7 124601); CLOCK (RsI801260, Rs934945, RsI3033501); Melatonin (any polymorphism); Orexin (all polymorphisms), PENK (RS16920581, RS1437277, RS1975285, RS260998, RS2609997), and CBI (RS1049353).

### Example 1

TABLE 1
Synaptamine ™ Gene Map for GnAP

| | | | INGREDIENT | |
|---|---|---|---|---|
| GENE NAME | POLYMORPHISM | PATHWAY(S) | CHANGE | REFERENCE(S) |
| Human kappa | In humans, the | The kappa opioid | DL- | [symbol] |
| opioid | 36G > T single | receptor (KOR) | Phenylalanine | Gerra G, |
| receptor gene | nucleotide | system seems to | L-Tyrosine | Leonardi C, Cortese E, |
| (OPRK1) | polymorphism | play a role in stress | Passion | D′Amore A, Lucchini A, |
| | (SNP) on KOR | responsivity, | Flower | Strepparola G, |
| | gene. | opiate with-drawal | | Serio G, Farina G, |
| | | and responses to | | Magnelli F, Zaimovic A, |
| | | psycho-stimulants, | | Mancini A, Turci M, |
| | | inhibiting | | Manfredini M, |
| | | mesolimbic | | Donnini C. |
| | | dopamine. KOR | | Human kappa opioid |
| | | gene poly- | | receptor gene |
| | | morphisms have | | (OPRK1) |
| | | been reported to | | polymorphism is |
| | | contribute to | | associated with |
| | | predisposition to | | opiate addiction. |
| | | voluntary alcohol- | | Am J Med Genet B |
| | | drinking behavior | | Neuropsychiatr |
| | | in experimental | | Genet. 2007 Sep |
| | | animals. | | 5; 144(6): 771-5. |
| Mu opioid | A118G SNP of the | Mu opioid | DL- | [symbol] |
| receptor | mu opioid | receptors are | Phenylalanine | Drakenberg K, |
| | receptor gene | critical for heroin | L-Tyrosine | Nikoshkov A, Horváth |
| | (OPRM1) | dependence, and | | MC, Fagergren P, |
| | | A118G SNP of the | | Gharibyan A, |
| | | mu opioid receptor | | Saarelainen K, |
| | | gene (OPRM1) has | | Rahman S, Nylander I, |
| | | been linked with | | Bakalkin G, Rajs J, |
| | | heroin abuse. In | | Keller E, Hurd YL. |
| | | our population of | | Mu opioid receptor |
| | | European | | A118G polymorphism |
| | | Caucasians (n = | | in association with |
| | | 118), | | striatal opioid |
| | | approximately 90% | | neuropeptide gene |
| | | of 118G allelic | | expression in heroin |
| | | carriers were | | abusers. |
| | | heroin users. | | Proc Natl Acad Sci USA. |
| | | | | 2006 May |
| | | | | 16; 103(20): 7883-8. |
| D(2) | A haplotype block | Within this block, | DL- | [symbol] |
| dopamine | of 25.8 kilobases | specific haplotype | Phenylanine | Xu K, Lichtermann D, |
| receptor gene | (kb) was defined | cluster A (carrying | L-Tyrosine | Lipsky RH, Franke P, |
| (DRD2) | by 8 SNPs | TaqIB1 allele) was | Passion | Liu X, Hu Y, Cao L, |
| | extending from | associated with a | Flower | Schwab SG, Wildenauer |
| | SNP3 (TaqIB) at | high risk of heroin | | DB, Bau CH, Ferro E, |
| | the 5′ end to | dependence in | | Astor W, Finch T, |
| | SNP10 site | Chinese patients (P = | | Terry J, Taubman J, |
| | (TaqIA) located | 1.425 × 10(−22); | | Maier W, Goldman D. |
| | 10 kb distal to the | odds ratio, 52.80; | | Association of specific |
| | 3′ end of the | 95% confidence | | haplotypes of D2 |
| | gene. | interval, 7.290-382.5 | | dopamine receptor |
| | | for 8-SNP | | gene with |
| | | analysis). A | | vulnerability to heroin |
| | | putative | | dependence in 2 |
| | | recombination | | distinct populations. |
| | | “hot spot” was | | Arch Gen Psychiatry. |
| | | found near SNP6 | | 2004 June; 61(6): 597-606. |
| | | (intron 6 ins/del | | [symbol] |
| | | G), creating 2 new | | Lawford BR, Young RM, |
| | | daughter | | Noble EP, Sargent J, |
| | | haplotypes that | | Rowell J, Shadforth S, |
| | | were associated | | Zhang X, Ritchie T. |
| | | with a lower risk of | | The D(2) dopamine |
| | | heroin | | receptor A(1) allele |
| | | dependence in | | and opioid |
| | | Germans (P = 1.94 × | | dependence: |
| | | 10(−11) for 8-SNP | | association with |
| | | analysis). | | heroin use and |
| | | Other studies show | | response to |
| | | the relationship of | | methadone |
| | | carrying TAq1A1 | | treatment. |
| | | vs. A2 alleles in the | | Am J Med Genet. |
| | | treatment | | 2000 Oct 9; 96 |
| | | outcomes for | | (5): 592-8. |
| | | heroin abuse. The | | Li Y, Shao C, Zhane D, |
| | | results indicate | | Zhao M, Lin L, Yan P, |
| | | that DRD2 variants | | Xie Y, Jiang K, Jin L. |
| | | are predictors of | | The effect of |
| | | heroin use and | | dopamine D2, D5 |
| | | subsequent | | receptor and |
| | | methadone | | transporter (SLC6A3) |
| | | treatment | | polymorphisms on |
| | | outcome and | | the cue-elicited |
| | | suggest a | | heroin craving in |
| | | pharmacogenetic | | Chinese. Am J Med |
| | | approach to the | | Genet B |
| | | treatment of | | Neuropsychiatr |
| | | opioid | | Genet. 2006; |
| | | dependence. | | 141(3): 269-73. |
| | | Others found | | |
| | | association | | |
| | | between nasal | | |
| | | inhalation of | | |
| | | opiates and DRD2 | | |
| | | promoter- | | |
| | | 141DeltaC | | |
| | | polymorphism. | | |
| | | Significantly | | |
| | | stronger cue- | | |
| | | elicited heroin | | |
| | | craving was found | | |
| | | in individuals | | |
| | | carrying D2 | | |
| | | dopamine receptor | | |
| | | gene (DRD2) Taql | | |
| | | RFLP A1 allele than | | |
| | | the non-carriers (P < | | |
| | | 0.001). | | |
| ANKK1 Gene | With a non- | Since DRD2 | L-Tyrosine | [symbol] [symbol] |
| | synonymous G to | expression is | | Huang W, Payne TJ, |
| | A transition, | regulated by | | Ma JZ, Beuten J, |
| | rs2734849 | transcription factor | | Dupont RT, Inohara N, |
| | produces an | NF-kappaB, we | | Li MD. |
| | amino-acid | suspect that | | Significant |
| | change (arginine | rs2734849 may | | Association of ANKK1 |
| | to histidine) in C- | indirectly affect | | and Detection of a |
| | terminal ankyrin | dopamine D (2) | | Functional |
| | repeat domain of | receptor density. | | Polymorphism with |
| | ANKK1. | The rs273849 | | Nicotine Dependence |
| | | ANNK1 variant the | | in an African- |
| | | alters expression | | American Sample. |
| | | level of NF- | | Neuropsychopharmacology. |
| | | kappaB-regulated | | 2008. |
| | | genes. | | |
| Catechol-O- | Val(108/158)Met | Genotyping 38 | L-Tyrosine | [symbol] |
| methyltransferase | polymorphism of | Israeli heroin | DL | Horowitz R, Kotler M, |
| (COMT) | the catechol-O- | addicts and both | Phenylalanine | Shufman E, Aharoni S, |
| gene | methyltransferase | parents using a | Rhodiola | Kremer I, Cohen H, |
| | (COMT) gene | robust family- | rosea | Ebstein RP. |
| | | based haplotype | | Confirmation of an |
| | | relative risk (HRR) | | excess of the high |
| | | strategy. There is | | enzyme activity |
| | | an excess of the val | | COMT val allele in |
| | | COMT allele | | heroin addicts in a |
| | | (likelihood ratio = | | family-based |
| | | 4.48, P = 0.03) and | | haplotype relative |
| | | a trend for an | | risk study. |
| | | excess of the | | Am J Med Genet. |
| | | val/val COMT | | 2000 Oct 9; 96(5): 599-603. |
| | | genotype | | [symbol] |
| | | (likelihood ratio = | | Cao L, Li T, Xu K, Liu X. |
| | | 4.97, P = 0.08, 2 df) | | Association study of |
| | | in the heroin | | heroin-dependence |
| | | addicts compared | | and −287 A/G |
| | | to the HRR control | | polymorphism of |
| | | group. | | catechol-O- |
| | | | | methyltransferase gene] |
| | | | | Zhonghua Yi Xue Yi |
| | | | | Chuan Xue Za Zhi. |
| | | | | 2002 December; 19(6): |
| | | | | 499-501. |
| Proenkephalin | > or = 81 by allele | Among the | DL- | Comings DE, Blake H, |
| gene (PENK) | | subjects with | Phenylalanine | Dietz G, Gade- |
| | | opioid | L-Tyrosine | Andavolu R, Legro RS, |
| | | dependence, 66% | Rhodiola | Saucier G, Johnson P, |
| | | carried the > or = | rosea | Verde R, MacMurray |
| | | 81 by allele | | JP. The |
| | | compared with | | proenkephalin gene |
| | | 40% of subjects | | (PENK) and opioid |
| | | with other types of | | dependence. |
| | | substance abuse | | Neuroreport. 1999 |
| | | (chi2 = 11.31, p < | | Apr 6; 10(5): 1133-5. |
| | | 0.004) and 49% of | | Nikoshkov A, |
| | | controls (chi2 = | | Drakenberg K, Wang X, |
| | | 6.0, p < 0.015). | | Horvath MC, Keller E, |
| | | These results are | | Hurd YL. Opioid |
| | | consistent with a | | neuropeptide |
| | | role of the PENK | | genotypes in relation |
| | | gene in opioid | | to heroin abuse: |
| | | dependence. | | dopamine tone |
| | | In another study, | | contributes to |
| | | Heroin abuse was | | reversed mesolimbic |
| | | significantly | | proenkephalin |
| | | associated with | | expression. Proc Natl |
| | | PENK polymorphic | | Acad Sci USA. 2008; |
| | | 3′ UTR | | 105(2): 786-91. |
| | | dinucleotide (CA) | | |
| | | repeats; 79% of | | |
| | | subjects | | |
| | | homozygous for | | |
| | | the 79-bp allele | | |
| | | were heroin | | |
| | | abusers. Such | | |
| | | individuals tended | | |
| | | to express higher | | |
| | | PENK mRNA than | | |
| | | the 81-bp | | |
| | | homozygotes, but | | |
| | | PENK levels within | | |
| | | the nucleus | | |
| | | accumbens (NAc) | | |
| | | shell were most | | |
| | | strongly correlated | | |
| | | to catecholamine- | | |
| | | O- | | |
| | | methyltransferase | | |
| | | (COMT) genotype. | | |
| | | Altogether, the | | |
| | | data suggest that | | |
| | | dysfunction of the | | |
| | | opioid reward | | |
| | | system is | | |
| | | significantly linked | | |
| | | to opiate abuse | | |
| | | vulnerability and | | |
| | | that heroin use | | |
| | | alters the apparent | | |
| | | influence of | | |
| | | heritable | | |
| | | dopamine tone on | | |
| | | mesolimbic PENK | | |
| | | and tyrosine | | |
| | | hydroxylase | | |
| | | function. | | |
| serotonin | Homozygosity at | Reward system | 5-hydroxy | Galeeva AR, Gareeva AE, |
| transporter | hSERT (especially | pathway | tryptophan | lur'ev EB, |
| (hSERT) | 10/10) was | | | Khusnutdinova EK. |
| | associated with | | | VNTR polymorphisms |
| | early opiate | | | of the serotonin |
| | addiction, while | | | transporter and |
| | genotype 12/10 | | | dopamine |
| | proved to be | | | transporter genes in |
| | protective. | | | male opiate addicts. |
| | | | | Mol Biol (Mosk). |
| | | | | 2002 36(4): 593-8 |
| | | | | Bonnet-Brilhault F, |
| | | | | Laurent C, Thibaut F, |
| | | | | Campion D, Chavand O, |
| | | | | Samolyk D, |
| | | | | Martinez M, Petit M, |
| | | | | Mallet J. Serotonin |
| | | | | transporter gene |
| | | | | polymorphism and |
| | | | | schizophrenia: an |
| | | | | association study. |
| | | | | Biol Psychiatry. |
| | | | | 1997; 42(7): 634-6. |
| Dopamine | In the case of | Reward System | DI- | Galeeva AR, Gareeva AE, |
| Transporter | DAT1, genotype | Pathway | Phenylalanine | lur'ev EB, |
| (DAT1) | 9/9 was | | L-Tyrosine | Khusnutdinova EK. |
| | associated with | | | VNTR polymorphisms |
| | early opiate | | | of the serotonin |
| | addiction. The | | | transporter and |
| | combination of | | | dopamine |
| | hSERT genotype | | | transporter genes in |
| | 10/10 with DAT1 | | | male opiate addicts. |
| | genotype 10/10 | | | Mol Biol (Mosk). |
| | was shown to be | | | 2002 36(4): 593-8 |
| | a risk factor of | | | |
| | opiate abuse | | | |
| | under 16 years of | | | |
| | age. | | | |
| Cannabinoid | A microsatellite | Cannabinoid | L-Glutamine | Comings DE, |
| CB1 (brain) | polymorphism | receptors in the | (decrease) | Muhleman D, Gade R, |
| receptor gene | (AAT)n at the | modulation of | L-Tyrosine | Johnson P, Verde R, |
| (CNR1) | cannabinoid CB1 | dopamine and | DL- | Saucier G, |
| | (brain) receptor | cannabinoid | Phenylalanine | MacMurray J. |
| | gene (CNR1) | reward pathways | | Cannabinoid receptor |
| | consists of 9 | | | gene (CNR1): |
| | alleles. The | | | association with i.v. |
| | number of i.v. | | | drug use. Mol |
| | drugs used was | | | Psychiatry. 2000 |
| | significantly | | | 5(2): 128-30. |
| | greater for those | | | |
| | carrying the > or | | | |
| | =/> or = 5 | | | |
| | genotype than | | | |
| | for other | | | |
| | genotypes (P = | | | |
| | 0.005). | | | |

P450 Liver Enzyme Gene

Polymorphisms

Common CYP2C8 and CYP2C9 polymorphisms and other polymorphisms (P450 GENE VARIANTS)

Pathway

Drug metabolism and pharmacogenomic response tied to narcotic drugs which will include any opiate used orally or in the transdermal form including Ketamine and even Gabapentin. Moreover these polymorphisms are also tied to NSAID metabolism and have been established as high risk gene polymorphisms for GI bleeds.

Action Required

Carriers of these polymorphisms (CYP2C8 and CYP2C9) will have a problem in metabolizing narcotics. Depending on the P450 polymorphism the physician will be required to either decrease or increase the said narcotic. Of equal importance the carriers of these polymorphisms will suggest NSAID GI risk in bleeding and thus the amount of NSAIDs used in the compounds will have to be adjusted accordingly. It is proposed that by increasing D-Phenylalanine we could have a natural anti-inflammatory response eliminating the need for high dosage NSAIDs.

Reference(s)

There are 10 studies relating polymorphisms of this gene and opiate response and there are over 20 studies involving NSAIDs GI bleed risk and P450 gene polymorphisms.

TNF-Alpha

Polymorphisms

TNF-alpha (−308(G-->A)), IL-10(−1082(G-->A))

Pathway

High risk for development of inflammatory secondary messengers. The carrying of the TNF-alpha polymorphism provides medical evidence for proper utilization of NSAIDs in the treatment of pain an inflammation. This includes any NSAID such as Ketoprofen, Baclofen, Cyclobenzapine, Diclofenac, Capsaicin, Ibuprofen. It is proposed that by increasing D-Phenylalanine we could have a natural anti-inflammatory response eliminating the need for high dosage NSAIDs.

Action Required

Carriers of the TNF-alpha polymorphism would require an increase in NSAIDs compounded in the pain ointment as prescribed the attending physician.

References

There are 2700 studies relating polymorphisms of this gene and the inflammatory response 3 studies specific to opiate response.

Nitric Oxide Gene (eNos)

Polymorphisms

−786T/C, −922A/G, 4B/4A, and 894G/T polymorphisms of eNOS

Pathway

Nitric oxide (NO) plays critical role in endothelial dysfunction and oxidative stress, pointing to the significance of endothelial nitric oxide synthase gene (eNOS) variants. Nitric Oxide deficiency leads to oxidative stress which prevents tissue healing. Furthermore, data imply that NMDA receptors and nitric oxide production in rostral ventromedial medulla modulate the transmission of opioid pain-inhibitory signals from the periaqueductal grey. It is proposed that by increasing Rhodiola rosea we could reduce oxidative stress. It is also proposed that by coupling the H-Wave device we could increase Nitric Oxide production as well.

Action Required

Carriers of the eNos gene polymorphisms will have an increased risk of slow healing due to oxidative stress. The physician will be required increase the amounts of pain medication and increase the number of prescriptions due to the reduced healing and the need to enhance the opioid pain-inhibitory responses.

References

There are 75 studies relating polymorphisms of this gene and oxidative stress. Additionally there are 21 papers showing the relationship of eNos polymorphisms and morphine actions related to pain inhibition.

Vascular Endothelial Growth Factor Gene (VEGF)

Polymorphisms

SNP genotypes, −160C, −152A (rs13207351), −116A (rs1570360

Pathway

Angiogenesis Factor-required for proper tissue healing these polymorphisms will slow the healing process. It has been demonstrated that there is a clear association between VEGF SNPs and severity of diabetic retinopathy. Furthermore, results suggested that endogenous opioid peptides (endomorphin-1 and -2 and deltorphin I) stimulated angiogenesis in the CAM assay, and these effects were modulated with the opioid receptors.

Action Required

Carriers of the VGEF gene polymorphisms will have an increased risk of slow healing due to lack of angiogenesis in the healing process. The physician will be required increase the amounts of pain medication and increase the number of prescriptions due to the reduced healing and the need to enhance the opioid pain-inhibitory responses by its induction of angiogenesis. A polymorphism in this gene will provide the medical necessity to prolong treatment past 30 days. It is also proposed that by coupling the H-Wave device we could increase angiogenesis as well.

References

There are 3423 studies relating polymorphisms of this gene and angiogenesis.
Dai X, Cui S G, Wang T, Liu Q, Song H J, Wang R. Endogenous opioid peptides, endomorphin-1 and -2 and deltorphin I, stimulate angiogenesis in the CAM assay. Eur J. Pharmacol. 2008 Jan. 28; 579(1-3):269-75.

### Example 2

Coupling RX pain compounds with Synaptamine and GeneMap

Gabapentin

Ketamine (C-111)-

Ketoprofan (KP)

Baclofen

Cyclobenzapine (antispasmodic agents)

Ibuprofen

Diclofenac

Capsaicin

Lidocaine

Menthol

Camphor

CX-659S

### Nimesulide Gel.

Novel Drug Delivery Systems

Soya-Lecithin Aggregates

In one study soya-lecithin aggregates, prepared by a technique using compressed gas, are used to formulate new dermal preparations. Ketoprofen (KP), a nonsteroidal anti-inflammatory drug (NSAID) is included as a model drug. The technique offers the possibility of incorporating auxiliary agents, such as penetration enhancers, anti-irritants and moisturizers together with the drug in one process. Apparent partition coefficients for n-octanol-phosphate buffer were determined for each of the lecithin aggregates. In general, soya-lecithin improves the partition of KP into n-octanol. The resulting products were included in widely used hydrophilic and hydrophobic vehicles. After 24 h, the cumulative amount of drug released through an artificial membrane was higher from the hydrophilic gels (2.6-4.3 mg) and the hydrophobic creams (0.23-0.392 mg) than from the control preparations (control hydrogel: 1.3 mg; control hydrophobic cream: 0.141 mg). However, the cumulative amount released from the hydrophobic vehicles was generally lower than from the hydrophilic matrices. Cumulative amounts such as those released from the hydrophilic preparations can also be achieved using supersaturated formulations based solely on the drug-loaded lecithin aggregates and a suitable oily component (4.07 mg). Results from the diffusion studies using artificial membranes were confirmed by permeation studies using excised rat skin. The improvement in skin permeation is related to both the solubilizing effect of the lecithin matrix and the penetration enhancing effect of lecithin itself. The novel soya-lecithin aggregates are promising candidates for new drug delivery systems in dermatology and cosmetology. Lecithin aggregates loaded with drugs are multifunctional carriers that also act as penetration enhancers.

Micronized

The bioavailability of S(+) and R(−) ketoprofen (KTP) in six horses was investigated after oral administration of the racemic (rac) mixture. Two oral formulations were studied, an oil-based paste containing micronized rac-KTP and powder from the same source in hard gelatin capsules, each at a dose rate of 2.2 mg/kg. For the oil-based paste two feeding schedules were used; horses were either allowed free access to food or access to food was restricted for 4 h before and 5 h after dosing. The drug in hard gelatin capsules was administered to horses with restricted access to food. After intravenous administration of rac-KTP, S(+) enantiomer concentrations exceeded those of the R(−) enantiomer. For S(+) and R(−)KTP, respectively, pharmacokinetic parameters were, t1/2 beta 0.99+/−0.14 h, 0.70+/−0.13 h; CIB 0.56+/−0.09, 0.92+/−0.20 L/h/kg; Vd(ss) 0.53+/−0.11, 0.61+/−0.10 L/kg. Following oral administration of rac-KTP as the oil-based paste to horses with free access to food, there were no detectable concentrations in plasma in three animals at any sampling time, while a fourth animal showed very low concentrations at two sampling times only. In the two remaining horses very low but detectable concentrations were present for 5 h. In the horses with restricted access to food, rac-KTP paste administration produced higher concentrations in plasma. However, bioavailability was very low, 2.67+/−0.43 and 5.75+/−1.48% for R(−) and S(+)KTP, respectively. When administered as pure drug substance in hard gelatin capsules, absorption of KTP was fairly rapid, but incomplete. Bioavailability was 50.55+/−10.95 and 54.17+/−9.9% for R(−) and S(+)KTP, respectively. This study demonstrates that rac-KTP had a modest bioavailability when administered as a micronized powder in hard gelatin capsules to horses with restricted access to food. When powder from the same source was administered as an oil-based paste, it was for practical purposes not bioavailable, regardless on the feeding schedule.

Cyclic Monoterpenes

The percutaneous absorption promoting effect and skin irritancy of cyclic monoterpenes were investigated in rats and with rabbits, respectively. Ketoprofen (KPF) was applied to rat skin in gel ointments containing various cyclic monoterpenes. Plasma concentrations of KPF markedly increased with the addition of the hydrocarbons of cyclic monoterpenes such as trans-p-menthane and d-limonene, whereas no significant enhancing effect was observed in the cases of other terpenes such as l-menthol, l-menthone and 1,8-cineole. The lipophilicity of the enhancers seems the important factor in promoting penetration of KPF through the skin. The enhancing activity of d-limonene was found to be much higher than that of Azone. Irritancy of the hydrocarbons of cyclic monoterpenes and Azone to the skin was evaluated using a Draize scoring method with rabbits. No change was observed on the skin surface when ethanol containing 2% of the hydrocarbons was applied to the dorsal skin, though a slight edema and erythema were observed in the case of Azone. In particular, an obvious difference was observed in the erythema formation between Azone and the hydrocarbons of cyclic monoterpenes.

Cyclohexanone Derivatives

The promoting effect of cyclohexanone derivatives on the percutaneous absorption of ketoprofen and indomethacin from gel ointments was investigated in rats. Drug absorption was markedly enhanced by the addition of 2-tert-butylcyclohexanone. Promoting activities of 2,6-dimethyl and 4-tert-butylcyclohexanone were also observed, but their effects were significantly lower than that of the 2-tert-butyl derivative. The effect of side chain length at the 2-position of the cyclohexanone ring on the percutaneous absorption of these drugs was determined similarly using a series of 2-n-alkylcyclohexanones. Pronounced effects were observed in the case of 2-n-octylcyclohexanone, suggesting that a chain length of eight carbons is an important factor for absorption enhancement in this series. The extent of absorption enhancement was found to be an almost linear function of 2-n-octycyclohexanone concentrations in the range from 0 to 10%.

Generally, a procedure which can serve as a possible basis for the laboratory study of the topical effect of NSAID was investigated in rats or guinea pigs. The effect of NSAID was greatly influenced by physical characteristics of the preparation such as drug particle size, solubility, ointment base and concentration of drug. Moreover, it was also found to be affected by many technical factors such as animal fixation, drug application times and methods (rubbing times or occlusive dressing technique) and amounts applied which play an important role in topical preparation. The topical application of NSAID ointment (1% of indomethacin, ketoprofen or diclofenac sodium) markedly inhibited the paw edema by carrageenin in rats. The inhibitory activity was the same as that of steroidal ointment (0.12% betamethasone 17-valerate or 0.05% fluocinonide), but was less than that by oral administration of these NSAID. Also, the NSAID ointment obviously inhibited the ultraviolet erythema in guinea pigs and the swelling in the hind feet of adjuvant arthritic rats. The inhibitory activities of NSAID ointments on these inflammatory responses were almost the same as those obtained by oral administration of such NSAID and more potent than those of steroidal ointments. Furthermore, NSAID ointments increased the pain threshold in the inflamed foot as determined by the method of Randall and Selitto. The analgesic activity of NSAID ointment was more potent than that of steroidal ointment, but less than that of NSAID administered orally. On the other hand, neither the systemic effects such as decrease in weight of the adrenals and thymus which were noted when steroidal ointment was used, nor the gastrointestinal lesions which were found by oral administration of NSAID, were recognized in rats in which NSAID ointment was applied topically. The anti-inflammatory effects of NSAID ointment correlated well with the drug concentration at the site of inflammation. These findings suggest that NSAID ointment has a clinical use in the treatment of inflammatory diseases.

isosorbide Dinitrate Ointment

In complex regional pain syndrome type 1 (CRPS1) vascular changes occur from the initial, inflammatory event onto the trophic signs during chronicity of the disease, resulting in blood flow disturbances and marked temperature changes. Pharmacotherapeutic treatment is generally inadequate. To determine whether local application of the nitric oxide donor isosorbide dinitrate (ISDN) could cause vasodilation and thereby improve tissue blood distribution in the affected extremity a pilot study was performed by Groeneweg et al (2008). In a pilot study, 5 female patients with CRPS1 in one hand were treated with ISDN ointment 4 times daily during 10 weeks. As a primary objective videothermography was used to monitor changes in blood distribution in both the involved and contralateral extremities. Patients treated with ISDN showed an increase of 4 degrees C. to 6 degrees C. in mean skin temperature of the cold CRPS1 hands, reaching values similar to that of the contralateral extremities within 2 to 4 weeks time, suggesting normalization of blood distribution. This was confirmed by an improvement in skin color. In 3 patients the Visual Analog Scale pain declined, whereas in the other 2 patients the Visual Analog Scale pain was unchanged over time. In the pilot study, topical application of ISDN seems to be beneficial to improve symptoms for patients with cold type CRPS1, but further study is needed.

Liopoderm. This substance increases absorption but there are no PUBMED published reports.

To the inventors knowledge this is the first unobvious proposed invention to couple the polymorphic genes with specific customized pain ointment compounds (described below). These genes will be explored in terms of their relationship to nutrients.

Synaptamine™

The combining of the Synaptamine complex protected by U.S. Pat. No. 724 with any compounded pain ointment would have a number of important benefits.

The minimum ingredient complex comprising of:

Rhodiola rosea

DL-Phenylalanine

Chromium salts/1-tryptophan

However and advanced formula includes Passion flower and a source of vitamin B12 and calcium, magnesium and potassium.

Literature Sample Support

The inventors are providing specific studies published to validate efficacy of individual ingredients utilized in the patented complex Synapatamine.™ When combined with Passion Flower and AlgaeCal as proposed in the advanced formula it is noteworthy that since the combination of subsequent ingredients have not been reported to date the combination cannot be considered obvious.

Rhodiola rosea
Jafari M, Feigner J S, Bussel I I, Hutchili T, Khodayari B, Rose M R, Vince-Cruz C, Mueller L D.
Rhodiola: a promising anti-aging Chinese herb. Rejuvenation Res. 2007 December; 10(4):587-602

Using the fruit fly, Drosophila melanogaster, we investigated the effects of Rhodiola on life-span. Rhodiola is a plant root used in traditional Chinese medicine that may increase an organism's resistance to stress. It has been proposed that Rhodiola can extend longevity and improve health span by alleviating oxidative stress.
Zhang L, Yu H, Sun Y, Lin X, Chen B, Tan C, Cao G, Wang Z. Protective effects of salidroside on hydrogen peroxide-induced apoptosis in SH-SY5Y human neuroblastoma cells. Eur J. Pharmacol. 2007 Jun. 14; 564(1-3):18-25.

Salidroside, a phenylpropanoid glycoside isolated from Rhodiola rosea L, shows potent antioxidant property. The mechanisms by which salidroside protected neuron cells from oxidative stress included the induction of several antioxidant enzymes, thioredoxin, heme oxygenase-1, and peroxiredoxin-1; the down regulation of pro-apoptotic gene Bax and the up regulation of anti-apoptotic genes Bcl-2 and Bcl-X(L). Furthermore, salidroside dose-dependently restored H2O2-induced loss of mitochondrial membrane potential as well as the elevation of intracellular calcium level. These results suggest that salidroside has protective effects against oxidative stress-induced cell apoptosis, which might be a potential therapeutic agent for treating or preventing neurodegenerative diseases implicated with oxidative stress.
Kim S H, Hvun S H, Choung S Y. Antioxidative effects of Cinnamomi cassiae and Rhodiola rosea extracts in liver of diabetic mice. Biofactors. 2006; 26(3):209-19

Both Cinnamomi cassiae and Rhodiola rosea extracts are used as anti-diabetic folk medicines. Recently, increased oxidative stress was shown to play an important role in the etiology and pathogenesis of diabetes mellitus and its complications. This study was designed to examine the effects of Cinnamomi cassiae and Rhodiola rosea extracts on blood glucose, lipid peroxidation, the level of reduced glutathione and its related enzymes (glutathione reductase, glutathione S-transferase), and the activity of the antioxidant enzymes (catalase, superoxide dismutase and glutathione peroxidase) in the liver of db/db mice. Diabetic C57BL/Ks db/db mice were used as experimental models. Cinnamomi cassiae and Rhodiola rosea extracts may be effective for correcting hyperglycemia and preventing diabetic complications.
Kanupriva, Prasad D, Sai Ram M, Kumar R, Sawhney R C, Sharma S K, Ilavazhagan G, Kumar D, Baneree P K. Cytoprotective and antioxidant activity of Rhodiola imbricata against tert-butyl hydroperoxide induced oxidative injury in U-937 human macrophages. Mol Cell Biochem. 2005 July; 275(1-2):1-6.

The present study reports cytoprotective and antioxidant activity of aqueous and alcoholic extracts of Rhodiola imbricata rhizome on tert-butyl hydroperoxide (tert-BHP) induced cytotoxicity in U-937 human macrophages. Both aqueous and alcoholic extracts of Rhodiola rhizome at a concentration of 250 microg/ml were found to inhibit tert-BHP induced free radical production, apoptosis and to restore the anti-oxidant levels to that of the control cells.
Battistelli M, De Sanctis R, De Bellis R, Cucchiarini L, Dachá M, Gobbi P. Rhodiola rosea as antioxidant in red blood cells: ultrastructural and hemolytic behaviour. Eur J Histochem. 2005 July-September; 49(3):243-54

The aim of the present study was to investigate the effect of the R. rosea roots aqueous extract on in vitro human erythrocytes exposed to hypochlorous acid (HOCl)-oxidative stress.
Arora R, Chawla R, Sagar R, Prasad J, Singh S, Kumar R, Sharma A, Singh S, Sharma R K. Evaluation of radioprotective activities Rhodiola imbricata Edgew—a high altitude plant. Mol Cell Biochem. 2005 May; 273(1-2):209-23.

The present study reports the radioprotective properties of a hydro-alcoholic rhizome extract of Rhodiola imbricata (code named REC-7004), a plant native to the high-altitude Himalayas.
De Sanctis R, De Bellis R, Scesa C, Mancini U, Cucchiarini L, Dachá M. In vitro protective effect of Rhodiola rosea extract against hypochlorous acid-induced oxidative damage in human erythrocytes. Biofactors. 2004; 20(3):147-59

Rhodiola rosea L. (Crassulaceae) is a plant living at high altitudes in Europe and Asia. Our study demonstrates that R. rosea is able to significantly protect, in a dose-dependent manner, human RBC from glutathione (GSH) depletion, glyceraldehyde-3-phosphate dehydrogenase (GAPDH) inactivation and hemolysis induced by the oxidant. The protection on GSH afforded by the R. rosea extract with respect to ascorbic acid, occurred also if added 2 or 5 min. later than the oxidant, suggesting a more rapid or powerful effect.
Wing S L, Askew E W, Luetkemeier M J, Ryan D T, Kamimori G H, Grissom C K. Lack of effect of Rhodiola or oxygenated water supplementation on hypoxemia and oxidative stress. Wilderness Environ Med. 2003 Spring; 14(1):9-16.

This study investigated the effects of 2 potentially “oxygen promoting” dietary supplements on hypoxia and oxidative stress at a simulated altitude of 4600 m.
Mook-Jung I, Kim H, Fan W, Tezuka Y, Kadota S, Nishi o H, Jung M W. Neuroprotective effects of constituents of the oriental crude drugs, Rhodiola sacra, R. sachalinensis and Tokaku-joki-to, against beta-amyloid toxicity, oxidative stress and apoptosis. Biol Pharm Bull. 2002 August; 25(8):1101-4

We tested the constituents of two Rhodiola plants, Rhodiola sacra S. H. Fu and R. sachalinensis A. BOR, and an Oriental crude drug, Tokaku-joki-to, for their neuroprotective effects. These results suggest that some of the tested compounds protect neurons from beta-amyloid toxicity based on antiapoptotic and antioxidative activity.
Boon-Niermeiier E K, van den Berg A, Wikman G, Wiegant F A. Phyto-adaptogens protect against environmental stress-induced death of embryos from the freshwater snail Lymnaea stagnalis. Phytomedicine. 2000 October; 7(5):389-99.

The main purpose of the studies presented in this paper is twofold: 1) to evaluate whether phyto-adaptogens (Acanthopanax senticosus and Rhodiola rosea) are able to exert a protective action against stress-induced death of embryos of the pond snail Lymnaea stagnalis; and 2) whether a possible protective action by phyto-adaptogens can be explained by the induction of heat shock proteins. Both Acanthopanax and Rhodiola exert a strong protective action against a lethal heat shock. In summary, there appears to be a difference in efficiency in enhancing resistance to the various stress conditions used (heat shock>menadione>copper>cadmium). Based on the results presented in this paper, we can conclude that phyto-adaptogens are able to enhance the resistance against the different stress conditions tested in developing individuals of Lymnaea.

D-Phenylalanine

Russell A L, McCarty M F. DL-phenylalanine markedly potentiates opiate analgesia—an example of nutrient/pharmaceutical up-regulation of the endogenous analgesia system. Med. Hypotheses. 2000 October; 55(4):283-8. In the author's clinical experience, concurrent treatment with DL-phenylalanine (DLPA) often appears to potentiate pain relief and also ease depression in patients receiving opiates for chronic non-malignant pain. Comprehensive support of the EAS with well-tolerated nutrients and pharmaceuticals may amplify the analgesic efficacy of chronic opiate therapy, while enabling dosage reductions that minimize opiate side-effects. Analogously, this approach may complement the efficacy of acupuncture and other analgesic measures that activate the EAS.
Litvinova S V, Shul'govski\check{i} VV, Gruden' M A, Panchenko L F, Terebilina N N, Aristova V V, Kaliuzhny\check{i} A L. [A comprehensive study of the neurochemical and immune mechanisms of morphine tolerance: the effects of naloxone]. Patol Fiziol Eksp Ter. 2000 January-March; (1):6-9.

It is concluded that naloxone in small doses can be used in patients to suppress morphine tolerance.
Solov'eva E V, Kulikov S V. Khar'kovski\check{i} A O, Bogdanov E G. The analgesic action of new enkephalin analogs. Eksp Klin Farmakol. 1994 November-December; 57(6):20-2. The enkephalin analogue peptide IKB-901 containing epsilon-ACA and cysteine with the modified S-end shows an analgetic activity in rats (1 micron, intrathecally and 5 mg/kg intravenously) and in cats (0.35 and 0.7 mg/kg intravenously). Naloxone (0.1 mg/kg) prevents the analgetic effect of peptide. The coadministration of the peptide and the enkephalinase inhibitor D-phenylalanine (0.35 and 10 mg/kg, respectively) enhances analgesia and displays an antihypertensive effect in nociceptive stimulation.
Litvinova S V, Kozlov Alu, Kaliuzhny\check{i} L V. The enkephalinase mechanisms of the resistance and tolerance to the analgesic effect of morphine in rats. Differences in the effects of the action of D-phenylalanine in morphine-sensitive, morphine-tolerant and morphine-resistant rats. Biull Eksp Biol Med. 1993 July; 116(7):54-6. It is suggested that morphine-resistant rats have a congenital and morphine-tolerant rats an acquired high level of enkephalinase activity which blocked the morphine analgetic action.
Dove B, Morgenstern E, Göres E. The analgesic action of d-phenylalanine in combination with morphine or methadone. Pharmazie. 1991 December; 46(12):875-7.

Combining D-Phe with narcotic analgesics already with doses inactive on separate application reduce some undesirable side effects like dependence, behavioural disorders and growth retardation are markedly lowered. These results suggest the possibility to design a combined drug similarly effective as well-introduced narcotic analgesics, but better tolerated.
Kaliuzhny\check{i} L V, Kozlov Alu. Action of an enkephalinase blocker on the effect of acupuncture in acupuncture sensitive and resistant rabbits. Biull Eksp Biol Med. 1991 December; 112(12):571-3. It's suggested that the recovery of pain sensibility after acupuncture analgesia is determined by enkephalinase's mechanism activation which is activated permanently in acupuncture-resistant rabbits.
Acupunct Electrother Res. 1991; 16(1-2):13-26.

Morphine analgesia mediated by activation of the acupuncture-analgesia-producing system.
Sato T, Takeshige C, Shimizu S.
Panocka I, Sadowski B. Potentiation of swim analgesia by D-amino acids in mice is genotype dependent. Pharmacol Biochem Behay. 1990 December; 37(4):593-6.

The effect of combined treatment with 125 mg/kg of D-phenylalanine plus 125 mg/kg of D-leucine (IP) on magnitude and duration of analgesia caused by 3 min swim at 20 degrees C. was studied in mouse lines selectively bred for 20 generations toward high and low level of stress-induced analgesia.
Ninomi a Y, Kawamura H, Nomura T, Uebavashi H, Sabashi K, Funakoshi M. Analgesic effects of D-amino acids in four inbred strains of mice. Comp Biochem Physiol C. 1990; 97(2):341-3. Prominent strain differences of mice were found in analgesic effects of D-amino acids. 2. In C57BL/6CrSlc and C3H/HeSlc mice, pain threshold, which was determined by using a hot-plate method, increased to 140-175% of the control after the systemic treatment of all three D-amino acids employed, such as D-phenylalanine, -leucine and -methionine, whereas in DBA/2CrSlc or BALB/cCrSlc mice, out of three only one D-amino acid, D-phenylalanine or -leucine, produced significant increase of pain threshold. 3.
Kitade T, Odahara Y, Shinohara S, Ikeuchi T, Sakai T, Morikawa K, Minamikawa M, Toyota S, Kawachi A, Hyodo M, et al. Studies on the enhanced effect of acupuncture analgesia and acupuncture anesthesia by D-phenylalanine (2nd report)—schedule of administration and clinical effects in low back pain and tooth extraction. Acupunct Electrother Res. 1990; 15(2):121-35. D-phenylalanine (DPA) administered as an inhibiting drug of this degrading enzyme might prolong analgesia induced by acupuncture.
Kitade T, Odahara Y, Shinohara S, Ikeuchi T, Sakai T, Morikawa K, Minamikawa M, Toyota S, Kawachi A, Hyodo M, et al. Studies on the enhanced effect of acupuncture analgesia and acupuncture anesthesia by D-phenylalanine (first report)—effect on pain threshold and inhibition by naloxone. Acupunct Electrother Res. 1988; 13(2-3):87-97.

DPA enhances the analgesic effect of acupuncture by the “endorphin mechanism.”
Iarosh A K, Goruk P S, Luk'ianov E A. Comparative characteristics of the functioning of brain structures exposed to morphine and D-phenylalanine. Farmakol Toksikol. 1987 March-April; 50(2):20-3.

In experiments on rats it was shown that morphine and D-phenylalanine in doses of 5 and 100 mg/kg, respectively, produce a similar by the degree increase of pain reaction thresholds at stimulation of paws through the electrified floor of the chamber.
Nurmikko T, Pertovaara A, Pöntinen P J. Attenuation of tourniquet-induced pain in man by D-phenylalanine, a putative inhibitor of enkephalin degradation. Acupunct Electrother Res. 1987; 12(3-4):185-91.

The results support some earlier reports suggesting that DPA has analgetic properties.
Xuan Y T, Shi Y S, Zhou Z F, Han J S. Studies on the mesolimbic loop of antinociception-II. A serotonin-enkephalin interaction in the nucleus accumbens. Neuroscience. 1986 October; 19(2):403-9.

We now report that antinociception induced by intra-periaqueductal gray injection of morphine can be attenuated also by the narcotic antagonist naloxone or the enkephalin antibodies administered into the nucleus accumbens, and potentiated by D-phenylalanine, a putative inhibitor of the degradation of enkephalins. Marcello F, Grazia S M, Sergio M, Federigo S. Pharmacological “enkephalinase” inhibition in man. Adv Exp Med. Biol. 1986; 198 Pt B:153-60.

“Enkephalinase”, a peptidase capable of degrading enkephalins, has been recently characterized in man, in both plasma and cerebro-spinal fluid (CSF). This study was designed to evaluate the ability of putative “enkephalinase” inhibitors, D-phenylalanine, captopril and thiorphan to decrease “enkephalinase” activity (EKA) in plasma and CSF in human sufferers. All drugs studied decreased plasma EKA. Captopril and thiorphan also decreased CSF EKA. Of the three drugs tested thiorphan proved to be the most potent “enkephalinase” inhibitor in both plasma and CSF. These results show the usefulness of EKA assessment as a procedure for evaluating the potency and specificity of putative “enkephalinase” inhibitors in man.
Ehrenpreis S. Analgesic properties of enkephalinase inhibitors: animal and human studies. Prog Clin Biol Res. 1985; 192:363-70.

D-phenylalanine, bacitracin and puromycin produce long-lasting, naloxone-reversible analgesia in mice. D-phenylalanine potentiates acupuncture analgesia in mice and humans and has been used to ameliorate a variety of human chronic pain conditions.
Ehrenpreis S. Pharmacology of enkephalinase inhibitors: animal and human studies. Acupunct Electrother Res. 1985; 10(3):203-8.

D-Phenylalanine (DPA), one of these enkephalinase inhibitors, has been used successfully for the management of chronic intractable pain in humans and to potentiate the treatment of many painful conditions by acupuncture. Other aspects of pharmacology of DPA will be discussed, including its effects on the cardio-vascular system, behavior, and lack of development of tolerance and dependence when used chronically in animals and humans.
Takeshige C. Differentiation between acupuncture and non-acupuncture points by association with analgesia inhibitory system. Acupunct Electrother Res. 1985; 10(3):195-202.

D-phenylalanine acts like a lesion of AIS in analgesia caused by stimulation of acupuncture and non-acupuncture points, and enhances naloxone reversible analgesia. The descending pain inhibitory system plays a role as the common pathway to produce these three kinds of analgesia. This pathway is found in the arcuate nucleus (dopaminergic), ventromedian nucleus of the hypothalamus, raphe nucleus (serotonergic), reticular gigantocellular nucleus (noradrenergic) and reticular paragigantocellular nucleus.
Bodnar R J, Butler P D. Modulation of deprivation-induced food intake by D-phenylalanine. Int J. Neurosci. 1983 September; 20(3-4):295-30.

D-phenylalanine has been shown to possess opiate-like effects upon pain perception. These results are discussed in terms of whether D-phenylalanine possesses direct or indirect opiate-like effects upon ingestion.
Kirchgessner A L, Bodnar R J, Pasternak G W. Naloxazone and pain-inhibitory systems: evidence for a collateral inhibition model. Pharmacol Biochem Behay. 1982 December; 17(6):1175-9.

Certain manipulations in rats such as hypophysectomy or D-phenylalanine injections decrease CWS analgesia while increasing morphine analgesia.
McKibbin L S, Cheng R S. Systemic D-Phenylalanine and D-Leucine for Effective Treatment of Pain in the Horse. Can Vet J. 1982 February; 23(2):39-40.

This study showed that subcutaneous injection of a solution of D-amino acids produced effective analgesia in horses.
Subst Alcohol Actions Misuse. 1982; 3(4):231-9.

D-phenylalanine and other enkephalinase inhibitors as pharmacological agents: implications for some important therapeutic application.

Ehrenpreis S.
Ehrenpreis S. D-phenylalanine and other enkephalinase inhibitors as pharmacological agents: implications for some important therapeutic application. Acupunct Electrother Res. 1982; 7(2-3):157-72.

A number of compounds have been shown to inhibit the degradation of enkephalins. One of these, D-phenylalanine, is also anti-inflammatory. D-phenylalanine has proven to be beneficial in many human patients with chronic, intractable pain. It is proposed the enkephalinase inhibitors may be effective in a number of human “endorphin deficiency diseases” such as depression, schizophrenia, convulsive disorders and arthritis. Such compounds may alleviate other conditions associated with decreased endorphin levels such as opiate withdrawal symptoms.
Donzelle G Bernard L, Deumier R Lacome M, Barre M, Lanier M, Mourtada M B. Curing trial of complicated oncologic pain by D-phenylalanine. Anesth Analg (Paris). 1981; 38(11-12):655-8.

Our data point out the consequences the enkephalinases inhibitors will take up for the cure of intractable cancer pain.
Bodnar R J, Lattner M Wallace M M. Antagonism of stress-induced analgesia by D-phenylalanine, an anti-enkephalinase. Pharmacol Biochem Behay. 1980 December; 13(6):829-33.

Administration of high (250 mg/kg) doses of D-phenylalanine retards the degradation process and elicits analgesia which is reversed by naloxone and which summates with electroacupuncture analgesia.
Cheng R S, Pomeranz B. A combined treatment with D-amino acids and electroacupuncture produces a greater analgesia than either treatment alone; naloxone reverses these effects. Pain. 1980 April; 8(2):231-6.

The D-amino acids (DAA), D-phenylalanine and D-leucine, produce naloxone reversible analgesia; electroacupuncture (EA) also produces analgesia which is blocked by naloxone. Combining the two treatments produces an additive effect with a larger analgesia than that produced by either treatment given alone; this combined effect is also blocked by naloxone.

Chromium Salts

Chromium salts are known enhancers of serotonin synthesis. This fact provides important inference that serotonergic activity being enhanced will influence pain mechanisms both peripheral and central. In this regard a PUBMED search resulted in 857 studies that coupled serotonin function and pain mechanisms.
Zhao Z Q, Gao Y J, Sun Y G, Zhao C S, Gereau R W 4th, Chen Z F. Central serotonergic neurons are differentially required for opioid analgesia but not for morphine tolerance or morphine reward. Proc Natl Acad Sci USA. 2007 Sep. 4; 104(36):14519-24. Epub 2007 Aug. 27.

The relationship between chromium and wound healing is direct but not necessarily as obvious as that of, say, zinc to wound healing. However, the ‘secret’ to the ‘Cr to wound healing relationship’ can be revealed by just understanding one simple fact. Cr improves insulin sensitivity AND insulin has a profound relationship to wound healing. Insulin resistance is directly related to wound (and diseased tissue) promoting disorders. There are many debilitating physical and mental maladies associated with advanced insulin-resistant (Met Synd X) disorders, like diabetes, chronic inflammation, increased infections, etc. Below is just one citation that references some mechanisms associated with insulin-resistance. So the Cr/wound healing relationship is irrefutable.
Hooper P L. Insulin Signaling, GSK-3, Heat Shock Proteins and the Natural History of Type 2 Diabetes Mellitus: A Hypothesis. Metab Syndr Relat Disord. 2007 September; 5(3):220-30.

Recognizing GSK-3 and Hsps in the pathogenesis of insulin resistance, the central common feature of the metabolic syndrome, and type 2 diabetes will expand our understanding of the disease, offering new therapeutic options.

L-Phenylalanine

L-Phenylalanine is the precursor of dopamine in the ventral tegmental are of the brain.
Hnasko T S, Sotak B N. Palmiter R D. Morphine reward in dopamine-deficient mice. Nature. 2005 Dec. 8; 438(7069):854-7.

In contrast, dopamine-deficient mice display a robust conditioned place preference for morphine when given either caffeine or I-dihydroxyphenylalanine (a dopamine precursor that restores dopamine throughout the brain) during the testing phases. Together, these data demonstrate that dopamine is a crucial component of morphine-induced locomotion, dopamine may contribute to morphine analgesia, but that dopamine is not required for morphine-induced reward as measured by conditioned place preference.
Leknes S, Tracey I. A common neurobiology for pain and pleasure. Nat Rev Neurosci. 2008 April; 9(4):314-20.

Recent molecular-imaging and animal studies have demonstrated the important role of the opioid and dopamine systems in modulating both pain and pleasure.
Scott D J, Stohler C S, Egnatuk C M, Wang H, Koeppe R A, Zubieta J K. Placebo and nocebo effects are defined by opposite opioid and dopaminergic responses. Arch Gen Psychiatry. 2008 February; 65(2):220-31.
Zhang Y, Xu M Y. Su J. Differential effects of dopamine on pain-related electric activities in normal rats and morphinistic rats. Neurosci Bull. 2007 May; 23(3):185-8.

Passion Flower
Dhawan K. Drug/substance reversal effects of a novel tri-substituted benzoflavone moiety (BZF) isolated from Passiflora incarnata Linn.—a brief perspective. Addict Biol. 2003 December; 8(4): 379-86

Because the BZF moiety isolated from P. incarnata is a tri-substituted derivative of alpha-naphthoflavone (7,8-benzoflavone), a well-known aromatase-enzyme inhibitor, the mode of action of BZF has been postulated to be a neurosteroidal mechanism vide in which the BZF moiety prevents the metabolic degradation of testosterone and upregulates blood—testosterone levels in the body. As several flavonoids (e.g. chrysin, apigenin) and other phytoconstituents also possess aromatase-inhibiting properties, and the IC50 value of such phytomoieties is the main factor determining their biochemical efficacy, by altering their chemical structures to attain a desirable IC50 value new insights in medical therapeutics can be attained, keeping in view the menace of drug abuse worldwide

Algaecal

Unpublished Data
Joel E. Michalek et al (2009)

In this Bone Health Report to the Nation, the US Surgeon General (SG) concluded that America's bone health is in jeopardy and issued a call to action for the development of bone health programs designed to increase health literacy, physical activity, and nutrition. To examine the safety and efficacy of a bone health plan that incorporated the three components recommended by the SG with two versions of a bone health supplement and examine the effects of compliance. Two groups of subjects who expressed an interest in improving their bone health were tested with Dual-energy X-ray Absorptiometry (DXA) and reviewed the AlgaeCal Bone Health Plan (the Plan), an original version of the bone health supplement, and the requirements of a 6-month open-labeled protocol. In the first group (Group 1), 274 potential subjects aged 18-85 expressed an interest in improving their bone health, 158 agreed to participate, and 125 completed the study per protocol (PP) completing DXA, blood chemistry and quality of life tests at baseline and 6 months later. Two weeks after the last subject in Group 1 completed the study, the same procedure was followed with a second group of 80 potential subjects (Group 2), 58 of whom volunteered and 51 completed PP following the same plan, but taking an revised version of the bone health supplement. The two supplements contained different amounts of a sea-algae calcium with multiple naturally-occurring magnesium and trace minerals, and supplemental magnesium, boron, and vitamins D-3, K-2, and C. There were no significant differences in mean baseline bone mineral density (BMD) between the two groups or in variables related to BMD (age, sex, height, weight, percent fat, fat mass, or lean mass). For both groups, no significant differences were found between volunteers and non-volunteers and those who completed PP and those who were lost to attrition with regard to variables related to BMD. As compared to the expected mean annualized percent change (MAPC), both groups experienced significant increases in MAPC above expected [Group 1: 1.2%, p=0.001; Group 2: 2.8%, p=0.001]. The MAPC from baseline in Group 1 (0.48%) was not significant (p=0.14), but the MAPC was significant in Group 2 (p<0.001) and the MAPC in Group 2 was significantly greater than that in Group 1 (p=0.005). The MAPC contrast between compliant and non-compliant subjects was significant in both Groups (p=0.001 and p=0.003 respectively) with compliant subjects increasing their MAPC more than non-compliant subjects. No clinically significant changes in blood chemistries or self-reported quality of life were found in either group Following the Plan as recommended for six months with either version of the bone health supplement was associated with improvements in mean annualized percentage change in BMD. Increased compliance facilitated greater increases as did modifying the bone health supplement with different amounts and types of nutrients, while holding all other components of the Plan constant.

### Preferred Embodiments

Sample Formulas for Pain Ointments

Each formulation consists of a base ointment cream containing a solubilizer (e.g. Soya-lecithin aggregates, Micronized, Cyclic monoterpenes, Cyclohexanone derivatives, isosorbide dinitrate and Lipoderm etc.). The ingredient percentages will vary dependent on genotype results. Base ointment (B0) constitutes just the base cream with the solubilizer. The range of dosing for each cream could be between 10 and 160 grams. The directions as per prescription would be to apply a thin layer to affected area 2-3 times a day. The table provides a matrix whereby each ingredient can either be compounded alone (just Bo) or with any of the listed ingredients as depicted in the matrix. Any and all combinations are applicable. It is understood that these pain compounds are to be used in conjunction with an electrotherapeutic device, preferably the H-wave (electronicwaveform Lab, Huntington Beach, Calif. This device is known to increase muscle microcirculation, induce Nitric Oxide as well as angiogenisis on chronic use to reduce pain and enhance the tissue healing process. However the copounds could be uysed without the anti-pain device.

| | | | | | | | | | | | | |
|---|---|---|---|---|---|---|---|---|---|---|---|---|
| D-Phenylalanine | BO | LID | MT | CAMP | GBP | KET | KEPF | CAP | DICLO | IBUF. | BAC | AM |
| L-Phenylalanine | BO | LID | MT | CAMP | GBP | KET | KEPF | CAP | DICLO | IBUF | BAC | AM |
| L-Glutamine | BO | LID | MT | CAMP | GBP | KET | KEPF | CAP | DICLO | IBUF | BAC | AM |
| L-5-Hydroxytryptophane | BO | LID | MT | CAMP | GBP | KET | KEPF | CAP | DICLO | IBUF | BAC | AM |
| Rhodiola rosea | BO | LID | MT | CAMP | GBP | KET | KEPF | CAP | DICLO | IBUF | BAC | AM |
| Chromium salt | BO | LID | MT | CAMP | GBP | KET | KEPF | CAP | DICLO | IBUF | BAC | AM |
| Pyridoxal-phosphate | BO | LID | MT | CAMP | GBP | KET | KEPF | CAP | DICLO | IBUF | BAC | AM |
| I-tyrosine | BO | LID | MT | CAMP | GBP | KET | KEPF | CAP | DICLO | IBUF | BAC | AM |
| Synaptamine complex | BO | LID | MT | CAMP | GBP | KET | KEPF | CAP | DICLO | IBUF | BAC | AM |
| Kyotorphin | BO | LID | MT | CAMP | GBP | KET | KEPF | CAP | DICLO | IBUF | BAC | AM |

Lidocaine (LID);
Menthanol (MT);
Camphor (CAMP);
Gabapentin (GBP);
Ketamine (KET);
Ketoprofen(KEPF);
Capsaicin (CAP);
Diclofenac(DICLO);
Ibuprofen (IBUF);
Baclofen (BAC);
Amitriptyline(AM);
Cyclobenzapine (CLB) all combinations.
Chromium salts include but limited to Picolinate, polynicotinate etc.

Sample additional combinations:

### Example 1

D-phenylalanine, LID, GBP, KET, KEPF (10/5/10/10/10%); D-Phenylanine, GBP, KET, BAC (10/10/10/4%); D-Phenylalanine, GBP, KET, LID (10/6/10/10%); D-Phenylanine, GBP, KET, AM, BAC(10/6/6/4/4%); D-Phenylalanine, KEPF (10/10%); D-Phenylalanine, KEPF (10/20%); D-Phenylalanine, KEPF, LID (10/10/5%); D-Phenylalanine, KEPF, CLB (10/20/2%); D-Phenylalanine, KEPF, LID, CLB (10/20/5/2%); D-Phenylalanine, IBUF, KEPF, CLB (10/10/10/1%); D-Phenylalanine, LiD (10/10%); D-Phenylalanine, DICLO (10/10%); D-phenylalanine, CAP, MT, CAMP (10/0.0375%); D-phenylalanine, CAP, MT, CAMP (10/05%); D-phenylalanine, KEPF, KET, CAP (10/10/6/0.075%).

### Example 2

L-phenylalanine, LID, GBP, KET, KEPF (10/5/10/10/10%); L-Phenylanine, GBP, KET, BAC (10/10/10/4%); L-Phenylalanine, GBP, KET, LID (10/6/10/10%); L-Phenylanine, GBP, KET, AM, BAC (10/6/6/4/4%); L-Phenylalanine, KEPF (10/10%); L-Phenylalanine, KEPF (10/20%); L-Phenylalanine, KEPF, LID (10/10/5%); L-Phenylalanine, KEPF, CLB (10/20/2%); L-Phenylalanine, KEPF, LID, CLB (10/20/5/2%); L-Phenylalanine, IBUF, KEPF, CLB (10/10/10/1%); L-Phenylalanine, LiD (10/10%); L-Phenylalanine, DICLO (10/10%); L-phenylalanine, CAP, MT, CAMP (10/0.0375%); L-phenylalanine, CAP, MT, CAMP (10/05%); L-phenylalanine, KEPF, KET, CAP (10/10/6/0.075%).

### Example 3

L-Glutamine, LID, GBP, KET, KEPF (10/5/10/10/10%); L-Glutamine, GBP, KET, BAC (10/10/10/4%); L-Glutamine, GBP, KET, LID (10/6/10/10%); L-Glutamine, GBP, KET, AM, BAC(10/6/6/4/4%); L-Glutamine, KEPF(10/10%); L-Glutamine, KEPF (10/20%); L-Glutamine, KEPF, LID (10/10/5%); L-Glutamine, KEPF, CLB(10/20/2%); L-Glutamine, KEPF, LID, CLB (10/20/5/2%); L-Glutamine, IBUF, KEPF, CLB (10/10/10/1%); L-Glutamine, LiD (10/10%); L-Glutamine, DICLO(10/10%); L-Glutamine, CAP, MT, CAMP (10/0.0375%); L-Glutamine, CAP, MT, CAMP (10/05%); L-Glutamine, KEPF, KET, CAP (10/10/6/0.075%).

### Example 4

5-HTP, LID, GBP, KET, KEPF (10/5/10/10/10%); 5-HTP, GBP, KET, BAC (10/10/10/4%); 5-HTP, GBP, KET, LID (10/6/10/10%); 5-HTP, GBP, KET, AM, BAC (10/6/6/4/4%); 5-HTP, KEPF (10/10%); 5-HTP, KEPF (10/20%); 5-HTP, KEPF, LID (10/10/5%); 5-HTP, KEPF, CLB (10/20/2%); 5-HTP, KEPF, LID, CLB (10/20/5/2%); 5-HTP, IBUF, KEPF, CLB (10/10/10/1%); 5-HTP, LiD (10/10%); 5-HTP, DICLO (10/10%); 5-HTP, CAP, MT, CAMP (10/0.0375%); 5-HTP, CAP, MT, CAMP (10/05%); 5-HTP, KEPF, KET, CAP (10/10/6/0.075%).

### Example 5

Rhodiola rosea, LID, GBP, KET, KEPF (10/5/10/10/10%); Rhodiola rosea, GBP, KET, BAC (10/10/10/4%); Rhodiola rosea, GBP, KET, LID (10/6/10/10%); Rhodiola rosea, GBP, KET, AM, BAC (10/6/6/4/4%); Rhodiola rosea, KEPF (10/10%); Rhodiola rosea, KEPF (10/20%); Rhodiola rosea, KEPF, LID (10/10/5%); Rhodiola rosea, KEPF, CLB (10/20/2%); Rhodiola rosea, KEPF, LID, CLB (10/20/5/2%); Rhodiola rosea, IBUF, KEPF, CLB (10/10/10/1%); Rhodiola rosea, LiD (10/10%); Rhodiola rosea, DICLO (10/10%); Rhodiola rosea, CAP, MT, CAMP (10/0.0375%); Rhodiola rosea, CAP, MT, CAMP (10/05%); Rhodiola rosea, KEPF, KET, CAP (10/10/6/0.075%).

### Example 6

Chromium salt, LID, GBP, KET, KEPF (0.01/5/10/10/10%); Chromium salt, GBP, KET, BAC (0.01/10/10/4%); Chromium salt, GBP, KET, LID (0.01/6/10/10%); Chromium salt, GBP, KET, AM, BAC(0.01/6/6/4/4%); Chromium salt, KEPF(0.01/10%); Chromium salt, KEPF (0.01/20%); Chromium salt, KEPF, LID (0.01/10/5%); Chromium salt, KEPF, CLB(0.01/20/2%); Chromium salt, KEPF, LID, CLB (0.01/20/5/2%); chromium salt, IBUF, KEPF, CLB (0.01/10/10/1%); Rhodiola rosea, LiD (0.01/10%); Chromium salt, DICLO(0.01/10%); Chromium salt, CAP, MT, CAMP (0.01/0.0375%); Chromium salt, CAP, MT, CAMP (0.01/05%); Chromium salt, KEPF, KET, CAP (0.01/10/6/0.075%).

### Example 7

Pyridoxal-phosphate, LID, GBP, KET, KEPF (0.05/5/10/10/10%); Pyridoxal-phosphate, GBP, KET, BAC (0.05/10/10/4%); Pyridoxal-phosphate, GBP, KET, LID (0.01/6/10/10%); Pyridoxal-phosphate, GBP, KET, AM, BAC (0.05/6/6/4/4%); Pyridoxal-phosphate, KEPF(0.05/10%); Pyridoxal-phosphate, KEPF (0.05/20%); Pyridoxal-phosphate, KEPF, LID (0.05/10/5%); Pyridoxal-phosphate, KEPF, CLB (0.05/20/2%); Pyridoxal-phosphate, KEPF, LID, CLB (0.01/20/5/2%); Pyridoxal-phosphate, IBUF, KEPF, CLB (0.01/10/10/1%); Rhodiola rosea, LiD (0.01/10%); Pyridoxal-phosphate, DICLO (0.05/10%); Pyridoxal-phosphate, CAP, MT, CAMP(0.05/0.0375%); Pyridoxal-phosphate, CAP, MT, CAMP (0.05/05%); Pyridoxal-phosphate, KEPF, KET, CAP (0.05/10/6/0.075%).

### Example 8

L-Tyrosine, LID, GBP, KET, KEPF (10/5/10/10/10%); L-Tyrosine, GBP, KET, BAC (10/10/10/4%); L-Tyrosine, GBP, KET, LID (10/6/10/10%); L-Tyrosine, GBP, KET, AM, BAC(10/6/6/4/4%); L-Tyrosine, KEPF(10/10%); L-Tyrosine, KEPF (10/20%); L-Tyrosine, KEPF, LID (10/10/5%); L-Tyrosine, KEPF, CLB(10/20/2%); L-Tyrosine, KEPF, LID, CLB(10/20/5/2%); L-Tyrosine, IBUF, KEPF, CLB (10/10/10/1%); L-Tyrosine, LID (10/10%); L-Tyrosine, DICLO(10/10%); L-Tyrosine, CAP, MT, CAMP(10/0.0375%); L-Tyrosine, CAP, MT, CAMP(10/05%); L-Tyrosine, KEPF, KET, CAP (10/10/6/0.075%).

### Example 9

Synaptamine, LID, GBP, KET, KEPF (10/5/10/10/10%); Synaptamine, GBP, KET, BAC (10/10/10/4%); Synaptamine, GBP, KET, LID (10/6/10/10%); Synaptamine, GBP, KET, AM, BAC(10/6/6/4/4%); Synaptamine, KEPF(10/10%); Synaptamine, KEPF (10/20%); Synaptamine, KEPF, LID (10/10/5%); Synaptamine, KEPF, CLB (10/20/2%); Synaptamine, KEPF, LID, CLB (10/20/5/2%); Synaptamine, IBUF, KEPF, CLB (10/10/10/1%); Synaptamine, LID (10/10%); Synaptamine, DICLO (10/10%); Synaptamine, CAP, MT, CAMP (10/0.0375%); Synaptamine, CAP, MT, CAMP (10/05%); Synaptamine, KEPF, KET, CAP (10/10/6/0.075%).

### Example 10

Kyotorphin, Synaptamine, LID, GBP, KET, KEPF (10/5/10/10/10%); Kyotorphin, Synaptamine, GBP, KET, BAC (10/10/10/4%); Kyotorphin, Synaptamine, GBP, KET, LID (10/6/10/10%); Synaptamine, GBP, KET, AM, BAC (10/6/6/4/4%); Kyotorphin, Synaptamine, KEPF (10/10%); Kyotorphin, Synaptamine, KEPF (10/20%); Kyotorphin, Synaptamine, KEPF, LID (10/10/5%); Kyotorphin, Synaptamine, KEPF, CLB (10/20/2%); Kyotorphin, Synaptamine, KEPF, LID, CLB (10/20/5/2%); Kyotorphin, Synaptamine, IBUF, KEPF, CLB (10/10/10/1%); Kyotorphin, Synaptamine, LID (10/10%); Kyotorphin, Synaptamine, DICLO (10/10%); Kyotorphin, Synaptamine, CAP, MT, CAMP (10/0.0375%); Kyotorphin, Synaptamine, CAP, MT, CAMP (10/05%); Kyotorphin, Synaptamine, KEPF, KET, CAP (10/10/6/0.075%).

### Example 10

Kyotorphin, LID, GBP, KET, KEPF (10/5/10/10/10%); Kyotorphin, GBP, KET, BAC (10/10/10/4%); Kyotorphin, GBP, KET, LID (10/6/10/10%); Kyotorphin, GBP, KET, AM, BAC (10/6/6/4/4%); Kyotorphin, KEPF (10/10%); Kyotorphin, KEPF (10/20%); Kyotorphin, KEPF, LID (10/10/5%); Kyotorphin, KEPF, CLB (10/20/2%); Kyotorphin, KEPF, LID, CLB (10/20/5/2%); Kyotorphin, IBUF, KEPF, CLB (10/10/10/1%); Kyotorphin, LID (10/10%); Kyotorphin, DICLO (10/10%); Kyotorphin, CAP, MT, CAMP (10/0.0375%); Kyotorphin, CAP, MT, CAMP (10/05%); Kyotorphin, KEPF, KET, CAP (10/10/6/0.075%).

Refereed Gene Map for Pain Ointments:

| | | | INGREDIENT | |
|---|---|---|---|---|
| GENE NAME | POLYMORPHISM | PATHWAY(S) | CHANGE | REFERENCE(S) |
| Human kappa | In humans, the | The kappa opioid | DL- | [symbol] |
| opioid receptor | 36G > T single | receptor (KOR) | Phenylalanine | Gerra G, Leonardi |
| gene (OPRK1) | nucleotide | system seems to | L-Tyrosine | C, Cortese E, |
| | polymorphism | play a role in | Passion Flower | D'Amore A, |
| | (SNP) on KOR | stress | | Lucchini A, |
| | gene. | responsivity, | | Strepparola G, |
| | | opiate | | Serio G, Farina G, |
| | | withdrawal and | | Magnelli F, |
| | | responses to | | Zaimovic A, |
| | | psycho- | | Mancini A, Turci M, |
| | | stimulants, | | Manfredini M, |
| | | inhibiting | | Donnini C. |
| | | mesolimbic | | Human kappa |
| | | dopamine. KOR | | opioid receptor |
| | | gene | | gene (OPRK1) |
| | | polymorphisms | | polymorphism is |
| | | have been | | associated with |
| | | reported to | | opiate addiction. |
| | | contribute to | | Am J Med Genet |
| | | predisposition to | | B Neuropsychiatr |
| | | voluntary | | Genet. 2007 Sep |
| | | alcohol-drinking | | 5; 144(6): 771-5. |
| | | behavior in | | |
| | | experimental | | |
| | | animals. | | |
| Mu opioid | A118G SNP of the | Mu opioid | DL- | [symbol] |
| receptor | mu opioid | receptors are | Phenylalanine | Drakenberg K, |
| | receptor gene | critical for heroin | L-Tyrosine | Nikoshkov A, |
| | (OPRM1) | dependence, and | | Horváth MC, |
| | | A118G SNP of the | | Fagergren P, |
| | | mu opioid | | Gharibyan A, |
| | | receptor gene | | Saarelainen K, |
| | | (OPRM1) has | | Rahman S, Nylander I, |
| | | been linked with | | Bakalkin G, Rajs J, |
| | | heroin abuse. In | | Keller E, Hurd YL. |
| | | our population of | | Mu opioid |
| | | European | | receptor A118G |
| | | Caucasians (n = | | polymorphism in |
| | | 118), | | association with |
| | | approximately | | striatal opioid |
| | | 90% of 118G | | neuropeptide |
| | | allelic carriers | | gene expression |
| | | were heroin | | in heroin |
| | | users. | | abusers. |
| | | | | Proc Natl Acad |
| | | | | Sci USA. 2006 |
| | | | | May |
| | | | | 16; 103(20): 7883-8. |
| D(2) dopamine | A haplotype | Within this block, | DL-Phenylanine | [symbol] |
| receptor gene | block of 25.8 | specific | L-Tyrosine | Xu K, Lichtermann D, |
| (DRD2) | kilobases (kb) | haplotype cluster | Passion Flower | Lipsky RH, Franke P, |
| | was defined by 8 | A (carrying | | Liu X, Hu Y, |
| | SNPs extending | TaqlB1 allele) | | Cao L, Schwab SG, |
| | from SNP3 | was associated | | Wildenauer DB, |
| | (TaqlB) at the 5′ | with a high risk of | | Bau CH, Ferro E, |
| | end to SNP10 site | heroin | | Astor W, Finch T, |
| | (TaqlA) located | dependence in | | Terry J, |
| | 10 kb distal to | Chinese patients | | Taubman J, |
| | the 3′ end of the | (P = 1.425 × 10(−22); | | Maier W, |
| | gene. | odds ratio, | | Goldman D. |
| | | 52.80; 95% | | Association of |
| | | confidence | | specific |
| | | interval, 7.290-382.5 | | haplotypes of D2 |
| | | for 8-SNP | | dopamine |
| | | analysis). A | | receptor gene |
| | | putative | | with vulnerability |
| | | recombination | | to heroin |
| | | “hot spot” was | | dependence in 2 |
| | | found near SNP6 | | distinct |
| | | (intron 6 ins/del | | populations. |
| | | G), creating 2 | | Arch Gen |
| | | new daughter | | Psychiatry. 2004 |
| | | haplotypes that | | June; 61(6): 597-606. |
| | | were associated | | [symbol] |
| | | with a lower risk | | Lawford BR, |
| | | of heroin | | Young RM, Noble EP, |
| | | dependence in | | Sargent J, Rowell J, |
| | | Germans (P = | | Shadforth S, |
| | | 1.94 × 10(−11) for | | Zhang X, Ritchie T. |
| | | 8-SNP analysis). | | The D(2) |
| | | Other studies | | dopamine |
| | | show the | | receptor A(1) |
| | | relationship of | | allele and opioid |
| | | carrying TAq1A1 | | dependence: |
| | | vs. A2 alleles in | | association with |
| | | the treatment | | heroin use and |
| | | outcomes for | | response to |
| | | heroin abuse. | | methadone |
| | | The results | | treatment. |
| | | indicate that | | Am J Med Genet. |
| | | DRD2 variants | | 2000 Oct 9; 96 |
| | | are predictors of | | (5): 592-8. |
| | | heroin use and | | Li Y, Shao C, |
| | | subsequent | | Zhang D, Zhao M, |
| | | methadone | | Lin L, Yan P, |
| | | treatment | | Xie Y, Jiang K, Jin L. |
| | | outcome and | | The effect of |
| | | suggest a | | dopamine D2, D5 |
| | | pharmacogenetic | | receptor and |
| | | approach to the | | transporter |
| | | treatment of | | (SLC6A3) |
| | | opioid | | polymorphisms |
| | | dependence. | | on the cue- |
| | | Others found | | elicited heroin |
| | | association | | craving in |
| | | between nasal | | Chinese. Am J |
| | | inhalation of | | Med Genet B |
| | | opiates and | | Neuropsychiatr |
| | | DRD2 promoter - | | Genet. 2006; |
| | | 141DeltaC | | 141(3): 269-73. |
| | | polymorphism. | | |
| | | Significantly | | |
| | | stronger cue- | | |
| | | elicited heroin | | |
| | | craving was | | |
| | | found in | | |
| | | individuals | | |
| | | carrying D2 | | |
| | | dopamine | | |
| | | receptor gene | | |
| | | (DRD2) Taql RFLP | | |
| | | A1 allele than the | | |
| | | non-carriers (P < | | |
| | | 0.001). | | |
| Catechol-O- | Val(108/158)Met | Genotyping 38 | L-Tyrosine | [symbol] |
| methyltransferase | polymorphism of | Israeli heroin | DL | Horowitz R, |
| (COMT) gene | the catechol-O- | addicts and both | Phenylalanine | Kotler M, Shufman E, |
| | methyltransferase | parents using a | Rhodiola rosea | Aharoni S, |
| | (COMT) gene | robust family- | | Kremer I, Cohen H, |
| | | based haplotype | | Ebstein RP. |
| | | relative risk | | Confirmation of |
| | | (HRR) strategy. | | an excess of the |
| | | There is an | | high enzyme |
| | | excess of the val | | activity COMT val |
| | | COMT allele | | allele in heroin |
| | | (likelihood ratio = | | addicts in a |
| | | 4.48, P = 0.03) | | family-based |
| | | and a trend for | | haplotype |
| | | an excess of the | | relative risk |
| | | val/val COMT | | study. |
| | | genotype | | Am J Med Genet. |
| | | (likelihood ratio = | | 2000 Oct |
| | | 4.97, P = 0.08, 2 | | 9; 96(5): 599-603. |
| | | df) in the heroin | | [symbol] |
| | | addicts | | Cao L, Li T, Xu K, |
| | | compared to the | | Liu X. |
| | | HRR control | | Association study |
| | | group. | | of heroin- |
| | | | | dependence and |
| | | | | −287 A/G |
| | | | | polymorphism of |
| | | | | catechol-O- |
| | | | | methyltransferase |
| | | | | gene] |
| | | | | Zhonghua Yi Xue |
| | | | | Yi Chuan Xue Za |
| | | | | Zhi. 2002 |
| | | | | Dec; 19(6): 499-501. |
| Proenkephalin | > or = 81 bp | Among the | DLPhenylalanine | Comings DE, |
| gene (PENK) | allele | subjects with | L-Tyrosine | Blake H, Dietz G, |
| | | opioid | Rhodiola rosea | Gade-Andavolu R, |
| | | dependence, | | Legro RS, |
| | | 66% carried the > | | Saucier G, |
| | | or = 81 bp allele | | Johnson P, Verde R, |
| | | compared with | | MacMurray JP. |
| | | 40% of subjects | | The |
| | | with other types | | proenkephalin |
| | | of substance | | gene (PENK) and |
| | | abuse (chi2 = | | opioid |
| | | 11.31, p < 0.004) | | dependence. |
| | | and 49% of | | Neuroreport. |
| | | controls (chi2 = | | 1999 Apr |
| | | 6.0, p < 0.015). | | 6; 10(5): 1133-5. |
| | | These results are | | Nikoshkov A, |
| | | consistent with a | | Drakenberg K, |
| | | role of the PENK | | Wang X, Horvath MC, |
| | | gene in opioid | | Keller E, |
| | | dependence. | | Hurd YL. Opioid |
| | | In another study, | | neuropeptide |
| | | Heroin abuse was | | genotypes in |
| | | significantly | | relation to heroin |
| | | associated with | | abuse: dopamine |
| | | PENK | | tone contributes |
| | | polymorphic 3′ | | to reversed |
| | | UTR dinucleotide | | mesolimbic |
| | | (CA) repeats; | | proenkephalin |
| | | 79% of subjects | | expression. Proc |
| | | homozygous for | | Natl Acad Sci USA. |
| | | the 79-bp allele | | 2008; |
| | | were heroin | | 105(2): 786-91. |
| | | abusers. Such | | |
| | | individuals | | |
| | | tended to | | |
| | | express higher | | |
| | | PENK mRNA than | | |
| | | the 81-bp | | |
| | | homozygotes, | | |
| | | but PENK levels | | |
| | | within the | | |
| | | nucleus | | |
| | | accumbens (NAc) | | |
| | | shell were most | | |
| | | strongly | | |
| | | correlated to | | |
| | | catecholamine- | | |
| | | O- | | |
| | | methyltransferase | | |
| | | (COMT) | | |
| | | genotype. | | |
| | | Altogether, the | | |
| | | data suggest that | | |
| | | dysfunction of | | |
| | | the opioid | | |
| | | reward system is | | |
| | | significantly | | |
| | | linked to opiate | | |
| | | abuse | | |
| | | vulnerability and | | |
| | | that heroin use | | |
| | | alters the | | |
| | | apparent | | |
| | | influence of | | |
| | | heritable | | |
| | | dopamine tone | | |
| | | on mesolimbic | | |
| | | PENK and | | |
| | | tyrosine | | |
| | | hydroxylase | | |
| | | function. | | |
| serotonin | Homozygosity at | Reward system | 5-hydroxy | Galeeva AR, |
| transporter | hSERT (especially | pathway | tryptophan | Gareeva AE, |
| (hSERT) | 10/10) was | | | lur'ev EB, |
| | associated with | | | Khusnutdinova EK. |
| | early opiate | | | VNTR |
| | addiction, while | | | polymorphisms |
| | genotype 12/10 | | | of the serotonin |
| | proved to be | | | transporter and |
| | protective. | | | dopamine |
| | | | | transporter |
| | | | | genes in male |
| | | | | opiate addicts. |
| | | | | Mol Biol (Mosk). |
| | | | | 2002 36(4): 593-8 |
| | | | | Bonnet-Brilhault F, |
| | | | | Laurent C, |
| | | | | Thibaut F, |
| | | | | Campion D, |
| | | | | Chavand O, |
| | | | | Samolyk D, |
| | | | | Martinez M, Petit M, |
| | | | | Mallet J. |
| | | | | Serotonin |
| | | | | transporter gene |
| | | | | polymorphism |
| | | | | and |
| | | | | schizophrenia: |
| | | | | an association |
| | | | | study. Biol |
| | | | | Psychiatry. |
| | | | | 1997; 42(7): 634-6. |
| Dopamine | In the case of | Reward System | Dl- | Galeeva AR, |
| Transporter | DAT1, genotype | Pathway | Phenylalanine | Gareeva AE, |
| (DAT1) | 9/9 was | | L-Tyrosine | lur'ev EB, |
| | associated with | | | Khusnutdinova EK. |
| | early opiate | | | VNTR |
| | addiction. The | | | polymorphisms |
| | combination of | | | of the serotonin |
| | hSERT genotype | | | transporter and |
| | 10/10 with DAT1 | | | dopamine |
| | genotype 10/10 | | | transporter |
| | was shown to be | | | genes in male |
| | a risk factor of | | | opiate addicts. |
| | opiate abuse | | | Mol Biol (Mosk). |
| | under 16 years of | | | 2002 36(4): 593-8 |
| | age. | | | |
| Cannabinoid CB1 | A microsatellite | Cannabinoid | L-Glutamine | Comings DE, |
| (brain) receptor | polymorphism | receptors in the | (decrease) | Muhleman D, |
| gene (CNR1) | (AAT)n at the | modulation of | L-Tyrosine | Gade R, Johnson P, |
| | cannabinoid CB1 | dopamine and | DL- | Verde R, |
| | (brain) receptor | cannabinoid | Phenylalanine | Saucier G, |
| | gene (CNR1) | reward pathways | | MacMurray J. |
| | consists of 9 | | | Cannabinoid |
| | alleles. The | | | receptor gene |
| | number of i.v. | | | (CNR1): |
| | drugs used was | | | association with |
| | significantly | | | i.v. drug use. Mol |
| | greater for those | | | Psychiatry. 2000 |
| | carrying the > or | | | 5(2): 128-30. |
| | =/> or = 5 | | | |
| | genotype than | | | |
| | for other | | | |
| | genotypes (P = | | | |
| | 0.005). | | | |

Added to the above genes the inventors propose that the following genes be added to the panel because of the potential involvement in tissue healing and inflammation: eNOS, TNF-alpha, VGF.

Dopamine and pain: A preferred embodiment

Background

It is well know that individuals respond differently to medications and certain nutraceuticals, in terms of both toxicity and treatment efficacy. Potential causes for such variability in drug (nutrient) effects include the pathogenesis and severity of the disease being treated: drug (nutrient) interactions; the individual's age, nutritional status; kidney and liver function; and concomitant illnesses. Despite the potential importance of these clinical variables in determining drug/nutrient effects, it is now recognized that inherited differences in the metabolism and disposition of drugs/nutrients, and genetic variants (polymorphisms) in the targets of drug/nutrient therapy (such as receptors like the dopamine D2 receptor), can have even greater influence on the efficacy and toxicity of either medications or nutraceuticals.

Dopamine and Pain: Brain Reward Cascade

Pain System

Our cutaneous nociceptive system clearly serves as an exteroceptive role in signaling potentially dangerous stimuli impinging upon our bodies, so that we can respond appropriately, depending upon the situational context. Our interoceptive nociceptive system signals tissues disorders (e.g. rheumatoid) that are essentially inescapable, and calls for responses more obviously in the homeostatic domain.

Mesolimbic dopamine in the suppression of tonic pain

These results indicate dopamine agonists that activate D2 receptors in the NAcc, inhibit inflammatory pain.

Dopamine D2 Receptors and Chronic Pain

Dopamine D2 receptors have been reported to mediate the inhibitory role of dopamine in animal models for persistent pain (Magnusson and Fisher, 2000). Hagelberg et. al. (2002), shown in healthy volunteers that high D2 receptor availability in the putamen is associated with low cold pain threshold and a high pain modulation capacity induced by conditioning stimulation. Furthermore, decreased [18F] FDOPA uptake and increased D2 receptor availability have been demonstrated in the putamen in a chronic orofacial pain state, the burning mouth syndrome (Hagelberg et. al. (2003).

Moreover, it was found that the increase in D2 receptor availability in the left putamen and the decrease in D1/D2 ratio imply that alterations in the striatal dopaminergic system as evaluated by PET may be involved in chronic orofacial pain conditions. In essence, we hypothesize that low or hypodopaminergic function in the brain may predispose individuals to low pain tolerance. Current research would support this concept and thus carriers of the D2 Taq A1 allele as observed in Reward Deficiency Syndrome (RDS) behaviors may be good candidates for nutrients or bioactive substances designed to enhance dopamine release in the brain.

Stress and Pain

The importance here is to understand that it is our position that indeed in an individual with chronic pain the subject is definitely in a stressful condition and therefore there is increased neuronal firing. There are numerous examples in the literature to support this contention. Furthermore, if a individual has the DRD2A1 variant, numerous studies have shown that resultant low dopamine D2 receptors caused an inability to cope with stress in the family and as an individual 11-13 (See Blum & Braverman 2001, Noble et. al, and Comings et. al.). In this regard, it is known that stress could even reduce D2 receptor mRNA message in the substantia nigra, the lateral part of the VTA, basal ganglia especially in the “reward site” the nucleus accumbens 14 (Dziedzicka-Wasylewska, 1997). This work supports the concept that forebrain dopamine systems are involved in mediating the behavioral effects of chronic mild stress. It further supports the view that in obese subjects (with chronic mild to moderate stress) with a compromised number of D2 receptor sites and reduced mRNA message, the firing frequency of a catecholaminergic neuron is enhanced and would be quite receptive to I-tyrosine supplementation as proposed in the formula. Moreover, it is also known that neuronal depletion of dopamine could also induce an independent end-product inhibitory state for TOH, which will also respond to I-tyrosine supplementation. With a slow release formula, there is constant dopamine release because of the effect of enhanced opioidergic activity via d-phenylalanine on substantia nigra GABA neurons.

Stress and Dopamine: Implications for the Pathophysiology of Chronic Widespread Pain

Exposure to stress can inhibit tonic pain and that intra-VTA morphine induces analgesia in theformalin test, suggest that the endogenous release of opioids in the VTA might be a mechanism underlying the stress-induced inhibition of tonic pain. Tonic pain maybe attenuated by dopamine D2 activation. It follows then that in this application we embrace as one inventive embodiment a natural method to cause a preferential release of dopamine in mesocorticolimbic pathways. In this regard, support of an attenuation of stress has be found with a variant of a complex with dopaminergic activation properties shown in one double-blind placebo controlled study (Blum et. al. 1989).

Fibromyalgia

One example of how stress and dopamine may interact involves fibromyalgia (FM) which has been called a “stress-related disorder” due to the onset and exacerbation of symptoms on the context of stressful events (Wood 2004). We are proposing that natural manipulation of the reward signaling and circuitry could become very commercially viable. Breaking of this cycle with a stress reducing substance, such as passion flower (see below) or the proposed Synaptamine which includes this substance.

### SUMMARY OF INVENTION

Most recently Li and his associates developed an addiction gene network that was constructed manually based on the common pathways identified in their 2008 study and protein interaction data. Addiction-related genes were represented as white boxes while neurotransmitters and secondary massagers were highlighted in purple. The common pathways are highlighted in green boxes. Related functional modules such as “regulation of cytoskeleton”, “regulation of cell cycle”, “regulation of gap junction”, and “gene expression and secretion of gonadotropins” were highlighted in carmine boxes. Several positive feedback loops were identified in this network. Fast positive feedback loops were highlighted in red lines and slow ones were highlighted in blue lines.

Drug addiction is a serious worldwide problem with strong genetic and environmental influences. Different technologies have revealed a variety of genes and pathways underlying addiction; however, each individual technology can be biased and incomplete. Li et al (2008) integrated 2,343 items of evidence from peer-reviewed publications between 1976 and 2006 linking genes and chromosome regions to addiction by single-gene strategies, microarray, proteomics, or genetic studies. Li et al (2008) identified 1,500 human addiction-related genes and developed KARG (http://karg.cbi.pku.edu.cn), the first molecular database for addiction-related genes with extensive annotations and a friendly Web interface. Li et al (2008) then performed a meta-analysis of 396 genes that were supported by two or more independent items of evidence to identify 18 molecular pathways that were statistically significantly enriched, covering both upstream signaling events and downstream effects. Five molecular pathways significantly enriched for all four different types of addictive drugs were identified as common pathways which may underlie shared rewarding and addictive actions, including two new ones, GnRH signaling pathway and gap junction. They connected the common pathways into a hypothetical common molecular network for addiction. They observed that fast and slow positive feedback loops were interlinked through CAMKII, which may provide clues to explain some of the irreversible features of addiction. Interestingly, the common thread involves dopaminergic genes.

The subsequent coupling of these and other genes relative to polymorphisms would allow for additional nutrient based nutrigenomic mapping. The combination will provide a map which will serve as a platform to derive novel DNA targeted areas which will link nutrients with potential anti-craving actions. Moreover, the inventors are also proposing that coupling of the Synaptamine complex and/or kyotorphin with outlined pain compounds into an ointment base with a known solubilizer is inventive an unobvious. Furthermore the coupling of this novel compounds with genotyping as suggested in the embodiment of this provisional application is inventive and unobvious as well. Both These areas are indeed novel, inventive and have not been accomplished heretofore.

What is claimed:

1-65. (canceled)

66. A method of treating a disease state or condition selected from the group consisting of a reward deficiency syndrome (RDS) behavior, a Substance Use Disorder (SUD), acute or chronic pain, inflammation, joint damage, stress, anxiety, sleep loss, insomnia, lethargy, attention deficit hyperactivity disorder, depression, and pre-menstrual dysphorric disorder, comprising administering to a patient determined to have a genotype correlated with the disease state or condition at least the following substances:
a. an opiate destruction-inhibiting amount of at least one substance selected from the group consisting of a D-amino-acid, a peptide, and a structural analogue or derivative of a D-amino-acid or a peptide;
b. a neurotransmitter synthesis-promoting amount of at least one neurotransmitter precursor selected from the group consisting of a dopamine precursor, optionally L-Tyr, L-Phe, or L-Dopa; a serotonin precursor, optionally L-Trp or 5-hydroxytyrptophane; and gamma amino butyric acid (GABA) precursor, optionally L-glutamine, l-glutamate or L-glutamic acid; and
c. a tryptophan concentration enhancing amount of at least one chromium salt; and
d. a catecholamine catalytic inhibitor of the enzyme Catecholamine o-methyl-transferase (COMT), optionally selected from the group consisting of any form of Rhodiola and Huperzine,
wherein the substances (a)-(d) are administered as part of one or more compositions.

67. A method according to claim 66 that further comprises administering at least one or more additional substances selected from the group consisting of:
a. a calming herbal component, optionally selected from the group consisting of passion flower or fruit, Black Currant Oil, Black Currant Seed Oil, Ribes nigrum, Borage Oil, Borage Seed Oil, Borago officinalis, Bovine Cartilage, Bromelain, Ananas comosus, Cat's Claw, Uncaria tomentosa, cetyl myristoleate, Cetyl-M, cis-9-cetylmyristoleate, Cmo, chondroitin culfate, collagen cydrolysate, collagen, gelatin, gelatine, gelatin hydrolysate, hydrolyzed [denatured] collagen, Devil's Claw, Devil's Claw Root, Grapple Plant, Wood Spider, Harpagophytum procumbens, Dhea—Dehydroepiandrosterone, Dimethyl Sulfoxide (DMSO), Evening Primrose Oil, Evening Primrose, Primrose, an Oenothera species (including Oenothera biennis), Feverfew, Tanacetum parthenium, Fish Oil, Flaxseed, Flaxseed Oil, Flax Oil, Linseed Oil, Linum usitatissimum, Ginger, Zingiber officinale, Gingko, Gingko biloba, Ginseng, American ginseng, panax quinquefolius, Asian ginseng, panax ginseng, Siberian ginseng, eleutherococcus senticosus, GLA (Gamma-ünolenic Acid), Glucosamine, Glucosamine sulfate, glucosamine hydrochloride, N-acetyl glucosamine, Gotu Kola, Gotu Cola, Brahmi, Brahma-Buti, Indian Pennywort, Centella asiatica, Grapeseed, Grapeseed Oil, Grapeseed Extract, Vitis vinifera, Green Tea, Chinese Tea, Camellia sinensis; Guggul, Gugulipid, Guggal, Commiphora mukul, Indian Frankincense, Frankincense, Boswellia, Boswellin, Salai Guggal, Boswellia serrata, Kava Kava, Kava, Kava Pepper, Tonga, Kava Root, Piper methysticum, melatonin, MsM (Methylsulfonylmethane), New Zealand Green-Lipped Mussel, Perna Canaliculus, Phellodendron Amurense, Sam-E (S-adenosyl-L-methione), shark cartilage, cartilage, St. John's Wort, Hypercium perforatum, Stinging Nettle, Urtica dioica, Thunder God Vine, Tripterygium wilfordii; Turmeric, Curcuma longa, Curcuma domestica, Type II Undenatured Chicken Collagen, Chicken Collagen, Chicken Type II Collagen, Type II Collagen, Valerian, Valeriana officianalis, White Willow, Willow Bark; SaNx Alba, White Willow Bark, Wild Yam, Discorea villosa, Ganoderma Lucidum, Mangosteen Extract, Quercetin, and a combination of any two or more of the foregoing; and/or
b. a vitamin component, optionally selected from the group consisting of Folic Acid, Vitamin D, Vitamin C, and Vitamin B₆, and a combination of any two or more of the foregoing vitamin components; and/or
c. mineral component, optionally selected from the group consisting of manganese, potassium, magnesium, calcium, coral calcium, Sierasil®, Algae Cal®, and any active salt thereof; and/or
d. a homeopathic component, optionally selected from the group consisting of Aceonite 12×; Belladonna 12×; Bryonia 12×; Chamonlia 6×; Ferrum Phos 12×; Gelsemium 12×; and Berberis 6×.

68. A method according to claim 66 that includes at least one of the following:
a. the D-amino-acid is selected from the group consisting of D-phenylalanine; D-Leucine; and hydrocinnamic acid; and/or
b. the neurotransmitter synthesis precursor is selected from the group consisting of a dopamine precursor, optionally L-Tyr, L-Phe, or L-dopa; a serotonin precursor, optionally L-Trp or 5-hydroxytryptophan; a gamma amino butyric acid (GABA) precursor, optionally L-glutamine, L-glutamic acid, or L-glutamate; an acetylcholine (ACH) or acetylcarnitine precursor, optionally L-choline or L-acetylcholine; L-carnitine; and aceyticarnitine; and/or
c. the chromium salt is selected from the group consisting of picolinate, polynicotinate, chloride, and any active salt thereof.

69. A method according to claim 66 comprising daily administration of:
a. approximately 32-10,000 mg of DL-phenylalanine, 10-10,000 mg of L-tyrosine, 5-5,000 mg of L-tryptophan, 3-30,000 mg of L-glutamine, 2-30,000 mg of chromium salt, 1-300 mg of pyridoxal-5′-phosphate, and 1-10,000 mg Rhodiola rosea; or
b. 2-2000 mg Passion flower; 5-1500 mg Kava Kava; 5-10,000 mg Rhodiola rosea; 5-10,000 mg Rhodendron; 5-10,000 mg DL-phenylalanine; 2-5000 mg L-tyrosine; 10-5,000 mg L-glutamine; 5-2000 mg 5-Hyroxytryptophane; 20-30,000 mg Chromium Picolinate or other active salt thereof; 1-1000 mg Pyridoxal phosphate; 1-1000 mg Vitamin B complex; 5-2000 mg Calcium citrate; 5-2000 mg Magnesium ascorbate; 10-20,000 mg Hydroxycitric acid (a potassium salt); and 2-2000 mg Magnolia.

70. A method according to claim 69(a) further comprising daily administration of 5-10,000 mg Algae Cal® and/or 5-10,000 mg Coral Calcium.

71. A method according to claim 66 wherein determination of whether a patient has a genotype correlated with the disease state or condition is made by performing an allelic analysis of nucleic acids, optionally DNA, obtained from a sample taken from a subject known or suspected to have the disease state or condition, wherein (i) the sample is optionally a buccal sample or a blood sample and/or (ii) the allelic analysis analyzes at least two genes to identity mutations in the genes, wherein identifying mutations optionally comprises measuring multiple genetic mutations through single nucleotide polymorphisms or gene expression.

72. A method according to claim 71 wherein the allelic analysis comprises identifying at least one mutation selected from the group consisting of: a polymorphism in a gene encoding a Beta-adrenergic receptor; a polymorphism in a gene encoding an angiotensin converting enzyme (ACE); a polymorphism in a gene encoding an angiotensin 11 TI receptor; a polymorphism in a gene encoding cholesteryl ester transfer protein; a polymorphism in a gene encoding a potassium channel; a polymorphism in a gene encoding a cytochrome P-450 enzyme, optionally CYP2D6; a polymorphism in a gene encoding a protein product of the HER2/neu oncogene; a polymorphism of the C825T gene; a polymorphism in the APOE gene locus); a polymorphism in the CT or TT allele of the dopamine D2 receptor gene; a SNP (polymorphism) designated AA, at nucleotide position−6 of the ANG gene; a polymorphism in a gene encoding Apo-A1; a polymorphism in a gene encoding Methylene Tetrahydrofolate Reductase (MTHFR), optionally a C677T polymorphism; a polymorphism in tumor necrosis factor (TNF) gene; a polymorphism in the carbohydrate responsive element-binding protein (ChREBP) gene; a polymorphism of the Leptin receptor gene; a polymorphism of the dopamine D2 receptors gene (DRD2); a polymorphism of any of the dopamine D1, D3, D4, and D5 genes; a dopamine D2 receptor polymorphism selected from the group consisting of Ser311cys and TaqIA; a polymorphism in a c-fos gene; a polymorphism in the c-jun gene; a polymorphism in the c-myc, gene; a polymorphism in a gene encoding Sterol Regulatory Element Protein-1 (SREBP-Ic); a polymorphism in a gene encoding mitochondrial glycerol-3-phosphate acetyltransferase gene (MGPAT); a polymorphism in a gene encoding the peroxisome proliferator-activated receptor PPAR-gamma-2) gene; the Prol2Ala polymorphism of the PPARgamma gene; a polymorphism in a gene encoding Tryptophan 2,3-Dioxygenase (TDO2); a polymorphism in a gene encoding TCP-I; a polymorphism in a gene encoding Mc4R; a polymorphism in a gene encoding CART; a polymorphism in a gene encoding interleukin-1 beta; a polymorphism in a gene encoding tumor necrosis factor-alpha; a polymorphism in a gene encoding an intracellular adhesion molecule; a polymorphism in a gene encoding interleukin-8, a polymorphism in a gene encoding and interleukin-10; a polymorphism in a gene encoding interferon-alpha; a polymorphism in a gene encoding Ras-Protein and (HLA-DRBI 0404 and OlOlor PTPN22 R620W); the Dopamine Receptor D3 Ser9Gly (−205-G/A, -7685-G/C) polymorphism; a polymorphism in a gene encoding Glutamine:fructose-6-phosphate amidotransferase (GFPTI or GFPT 2), optionally polymorphisms in exon 14, optionally 1471V, or 3′ UTR; or a polymorphism in a gene encoding glucosamine 6-P acyltransferase; a polymorphism in Aggrecan proteoglycan allele 27; a polymorphism in a gene encoding 11-beta hydroxysteroid dehydrogenase typel; a polymorphism in a gene encoding FK506 binding protein 5; a polymorphism in a gene encoding serum/glucosteroid kinase; a polymorphism in a gene encoding tryptophan 2,3 dioxygenase; a polymorphism in a gene encoding Myelin; a polymorphism in a gene encoding a Myelin associated glycoprotein, optionally myelin oligodendrocyte glycoprotein (MOG), optionally a polymorphism in a tetranucleotide TAAA repeat (MOG4), C10991T SNP; a polymorphism in a gene encoding Edg2; a polymorphism in a gene encoding Fgfr2; a polymorphism in a gene encoding Decorin; a polymorphism in a gene encoding Brevican; a polymorphism in a gene encoding Neurotensin (NT) receptors-1; a polymorphism in a gene encoding Neurotensin (NT) receptor-2; a polymorphism in a gene encoding Neurotensin (NT) receptor-3; a polymorphism in a gene encoding Proenkephalin; a polymorphism in a gene encoding prodynorphin, optionally 946C>G; a polymorphism in a gene encoding Bdnf (Neurotrophic Factor, optionally BDNF Va166Met and −281 C>A, T allele of the C270T); a polymorphism in a gene encoding Sgk (Serum- and glucose-regulated kinase (SGK 1), optionally SNP Intron 6, Exon 8 (CC, CT, TT); a polymorphism in a gene encoding Gabl; Id2; a polymorphism in a gene encoding COMT; a polymorphism in a gene encoding ANKKI; a polymorphism in a gene encoding DATI; a polymorphism in a gene encoding DBH; a polymorphism in a gene encoding HTT; a polymorphism in a gene encoding HTRIA; a polymorphism in a gene encoding HTRID; a polymorphism in a gene encoding HTR2A; a polymorphism in a gene encoding HTR2c, optionally 5-HT-2A, 5-HT 2B, 5-HT-4, and 5-HT-7); a polymorphism in a gene encoding ADRA2A; a polymorphism in a gene encoding ADRA2; a polymorphism in a gene encoding NET; a polymorphism in a gene encoding MAOA; a polymorphism in a gene encoding GABRA3; a polymorphism in a gene encoding GABRB3; a polymorphism in a gene encoding CNRI; a polymorphism in a gene encoding CNRA4; a polymorphism in a gene encoding NMDARI; a polymorphism in a gene encoding POMC; a polymorphism in a gene encoding MGPAT; a polymorphism in a gene encoding NYP; a polymorphism in a gene encoding AgRP; a polymorphism in a gene encoding OBR; a polymorphism in a gene encoding Mc3R:UCP-1; a polymorphism in a gene encoding GLUT4; a polymorphism in a gene encoding PDGS; a polymorphism in a gene encoding ALdB; a polymorphism in a gene encoding LNC2; a polymorphism in a gene encoding E23K Kir6.2; a polymorphism in a gene encoding steroid sulfatase (STS); a polymorphism G82G in PTPNI; the IVS6+G82A polymorphism; a polymorphism in a gene encoding Sulfonylurea receptor 1; a polymorphism in a gene encoding beta(3)-AR Trp64Arg; a polymorphism in a gene encoding PC1; a polymorphism in a GHRELIN gene; a polymorphism in a gene encoding FKBP5; a polymorphism in a gene encoding a VITAMIN D RECEPTOR, optionally BSMI AND FOKI; a polymorphism in a gene encoding lymphoid tyrosine phosphatase (LYP), optionally a polymorphism in a gene encoding protein tyrosine phosphatase-22 (PTPN22) gene, and a polymorphism in a gene encoding any sodium ATPAse.

73. A method according to claim 71 wherein the allelic analysis comprises identifying at least one mutation that is a polymorphism selected from the group consisting of a polymorphism (Rs value of SNP) of a gene encoding DRD2 (RsI800497, Rs6278, Rs6276, RsIO79594, Rs6275, RsI801028, RsI076560, Rs2283265, RsIO79727, RsIO76562, RsII25394, Rs4648318, Rs4274224, Rs7131056, Rs4648317, RsI799732, RsI799978; 5HT2A(Rs6314, Rs3742278, Rs6561333, RsI923886, Rs643627, Rs2770292, RsI928040, Rs2770304, Rs594242, Rs6313; ANKKI (RS2734849, RS1800497, RsII604671, Rs4938016); OPRKI (Rs35160174, Rs35373196, Rs34709943 RS6473797) OPRMI (Rs510769, Rs553202, Rs514980, Rs561720, Rs534673, Rs524731, Rs3823010, Rs3778148, Rs7773995, R5495491, RsI2333298, RsI461773, RsI381376, Rs3778151, Rs506247, Rs563649, Rs9479757, Rs2075572, RsI0485057, Rs540825, Rs562859, Rs548646, Rs648007, Rs9322447, Rs681243, Rs609148, Rs3798687, Rs648893); COMT (Rs737864, Rs933271, Rs5993882, Rs740603, Rs4646312, RsI65722, Rs6269, RsI7699); SLC6A3 (RsI2516948, RsI042098, Rs40184, RsII564773, RsIII33767, Rs6876225, Rs3776512, Rs2270912, Rs6347, Rs27048, Rs37022, Rs2042449, Rs464069, Rs463379, Rs403636, Rs2617605, RsI3189021, Rs6350, Rs2975223, Rs2963238, RsI1564752 Rs2975226); HTR3B(Rs3758987, Rs2276307, Rs3782025, RsI672717); NOS3 (Rs891512, RsI808593, Rs2070744, Rs3918226, Rs7830); PPARG (RsI801282, Rs2938392, RsI175542, RsI7036314, RsI805192, Rs4684847, Rs2938392, Rs709157, Rs709158, RsI175542); ChREBP(Rs3812316); FTO (Rs8050136, RsI421084, Rs9939609, RsI861868, Rs9937053, Rs9939973, Rs9940128, RsI558902, RsI0852521, RsI477196, RsI121980, Rs7193144, RsI6945088, Rs8043757, Rs3751812, Rs9923233, Rs9926289, RsI2597786, Rs7185735, Rs9931164, Rs9941349, Rs7199182, Rs9931494, RsI7817964, Rs7190492, Rs9930506, Rs9932754, Rs9922609, Rs7204609, Rs8044769, RsI2149832, Rs6499646, RsI421090, Rs2302673); TNFalpha (RsI799964, RsI800629, Rs361525, RsI800610, Rs3093662); MANEA (RsI133503); LeptinOb (Rs4728096, RsI2536535, Rs2167270, Rs2278815, RsI0244329, RsII763517, RsII760956, RsIO954173); PEMT (Rs4244593, Rs936108); MAO-A (Rs3788862, RsI465108, Rs909525, Rs2283724, RsI2843268, RsI800659, Rs6323, RsI799835, Rs3027400, Rs979606, Rs979605 RsI137070); CRH (Rs7209436, Rs4792887, RsI10402, Rs242924, Rs242941, Rs242940, Rs242939, Rs242938, RsI73365, RsI876831, RsI876828, Rs937, Rs878886 Rs242948); ADIPOQ (RsI7300539, Rs2241766); STS (RsI2861247); VDR(RsI7467825, Rs731236, RsI544410, Rs2229828, Rs2228570, Rs2238136); DBI (Rs3091405, Rs3769664, Rs3769662, Rs956309, Rs8192506); GABRA6 (Rs3811995, Rs3219151, Rs6883829, Rs3811991); GABRB3 (Rs2912582, Rs2081648, RsI426217, Rs754185, Rs890317, Rs981778, Rs2059574); MTHFR(Rs4846048, RsI801131, RsI801133, Rs2066470); MLXIPL[carbohydrate binding element] (Rs3812316, RsI7145738); VEGF (Rs2010963, Rs833068, Rs3025000, Rs3025010, Rs3025039, Rs3025053); DRD4 (Rs936460, Rs41298422, Rs3758653, Rs936461, RsI2720373, Rs747302, RsI800955, Rs916455, Rs916457, Rs7 124601); CLOCK (RsI801260, Rs934945, RsI3033501); Melatonin (any polymorphism); Orexin (all polymorphisms), PENK (RS16920581, RS1437277, RS1975285, RS260998, RS2609997), and CBI (RS1049353).

74. A method according to claim 66 wherein:
a. the disease state or condition is joint damage caused by rheumatoid arthritis and the determined genotype comprises allelic analysis of polymorphisms in the tumor necrosis factor (TNF) gene informs a differential response to fish oil supplementation for the treatment of rheumatoid arthritis; or
b. the disease state or condition is inflammation and the determined genotype comprises allelic analysis of polymorphisms in the tumor necrosis factor (TNF) gene informs a differential response to vitamin E for promoting anti-oxidant activity and reducing inflammatory processes; or
c. the disease state or condition is pain intolerance and the determined genotype comprises allelic analysis of polymorphisms in the dopamine D2 receptor gene informs a differential response to a chromium salt; or
d. the disease state or condition is pain and the determined genotype comprises allelic analysis of polymorphisms in a gene selected from the group consisting of the dopamine D2, D1, D3, D4, and D5 receptor genes is used to adjust a dosage of one or more of (i) the substance selected from the group consisting of a D-amino-acid, a peptide, and a structural analogue or derivative of a D-amino-acid or a peptide, (ii) the neurotransmitter precursor, (iii) the chromium salt, and/or (iv) the catecholamine catalytic inhibitor, for pain control; or
e. the determined genotype comprises allelic analysis of polymorphisms in the human TDO2 gene and informs adjusting dosage of L-tryptophan, 5-hydroxytryptophan, and/or a chromium salt; or
f. the determined genotype comprises allelic analysis of polymorphisms in a gene selected from the group consisting of the interleukin-1 alpha gene, interleukin-1 beta gene, the TNF-alpha gene, the intracellular adhesion molecule gene, the interleukin-8 gene, and the interleukin-10 gene informs adjusting dosage of Echinacea; or
g. the determined genotype comprises allelic analysis of polymorphisms in the Methylene Tetrahydrofolate Reductase (MTHFR) gene, optionally the C677T polymorphism, informs adjusting dosage of a vitamin, optionally, folic acid, also administered to the patient; or
h. the determined genotype comprises allelic analysis of polymorphisms in the hippocalcin like 1 (Hpcall) gene informs adjusting dosage of a mineral, optionally, calcium, also administered to the patient; or
i. the determined genotype comprises allelic analysis of polymorphisms in a gene selected from the group consisting of the proenkephalin gene, prodynorphin gene, neurotensin (1,2,3) gene, the Bdnf gene, the TD02 gene, the Sgk gene, the Fkbp5&4 gene, the Edg2 gene, the Id2 gene, and the Gabl Fgfr2 gene to informs adjusting dosage of an herbal component, optionally, Passion flower, also administered to the patient as part of the method; or
j. the determined genotype comprises allelic analysis of polymorphisms in a gene selected from the group consisting of the proenkephalin gene, prodynorphin gene, neurotensin (1,2,3) gene, the Bdnf gene, the TD02 gene, the Sgk gene, the Fkbp5&4 gene, the Edg2 gene, the Id2 gene, and the Gabl Fgfr2 gene to inform adjusting dosage of Rhodiola, optionally, Rhodiola rosea;
k. the determined genotype comprises allelic analysis of polymorphisms in a gene selected from the group consisting of the COMT gene, the proenkephalin gene, the prodynorphin gene, the neurotensin (1,2,3) gene, the Bdnf gene, the TD02 gene, the Sgk gene, the Fkbp5&4 gene, the Edg2 gene, and Id2 gene to informs adjusting dosage of Rhodendron that is also administered to the patient as part of the method;
l. the determined genotype comprises allelic analysis of polymorphisms in a gene selected from the group consisting of the COMT gene, the DRD1-5 gene, the ANKKI gene, the DATI gene, the DBH gene, the TD02 gene, the HTT gene, the HTRIA gene, the HTRID gene, the HTR2A gene, the HTR2c gene, the ADRA2A gene, the ADRA2 gene, the NET gene, the MAOA gene, the GABRA3 gene, the GABRB3 gene, the CNR1 gene, the CNRA4 gene, the NMDAR1 gene, the POMC gene informs adjusting dosage of dl-phenylalanine; or
m. the determined genotype comprises allelic analysis of polymorphisms in a gene selected from the group consisting of the COMT gene, the NET gene, the MAOA gene, any of the DRD1-5 genes, ANKKI gene, the DATI gene, the DBH gene, the POMC gene, the proenkephalin gene, the prodynorphin gene, the neurotensin (1 gene, the 2 gene, the3) Bdnf gene, the TD02 gene, the Sgk gene, the Fkbp5&4 gene, the Edg2 gene, the Id2 gene, and the GabI Fgfr2 gene informs adjusting dosage of L-Tyrosine; or
n. the determined genotype comprises allelic analysis of polymorphisms in a gene selected from the group consisting of the COMT gene, the NET gene, the MAOA gene, the POMC gene, the proenkephalin gene, the prodynorphin gene, any neurotensin (1, 2, or 3) gene, the GABRA3 gene, and the NMDAR1 gene the informs adjusting dosage of the neurotransmitter precursor, optionally L-glutamine; or
o. the determined genotype comprises allelic analysis of polymorphisms in a gene selected from the group consisting of the COMT gene, the NET gene, the MAOA gene, the POMC gene, the proenkephalin gene, the proenkephalin gene, the prodynorphin gene, any neurotensin (1, 2, or 3) gene, the TD02 gene, the HTT gene, the HTRIA gene, the HTRID gene, the HTR2A gene, and the HTR2c gene informs adjusting dosage of the neurotransmitter precursor, optionally 5-Hyroxytryptophane; or
p. the determined genotype comprises allelic analysis of polymorphisms in a gene selected from the group consisting of the COMT gene, the NET gene, the MAOA gene, the POMC gene, the proenkephalin gene, the prodynorphin gene, any neurotensin (1, 2, or 3) gene, the TD02 gene, the HTT gene, the HTRIA gene, the HTRID gene, the HTR2A gene, the HTR2c gene, the DRD1-5 gene, the ANKKI HTR2A gene, the HTR2c gene, the DRD1-5 gene, the ANKKI gene, the DATI gene, and the DBH gene informs adjusting dosage of a chromium salt; or
q. the determined genotype comprises allelic analysis of polymorphisms in a gene selected from the group consisting of the HTT gene, the HTRIA gene, the HTRID gene, the HTR2A gene, the HTR2c, the 5-HT-2A gene, the 5-HT 2B gene, the 5-HT-4 gene, the 5-HT-7 gene, the COMT gene, any of the DRD1-5 genes, the ANKKI gene, the DATI gene, the DBH gene, the TD02 gene, the ADRA2A gene, the ADRA2 NET gene, the MAOA gene, the GABRA3 gene, the GABRB3 gene, the CNR1 gene, the CNRA4 gene, the NMDAR1 gene, the POMC gene, the proenkephalin gene, the prodynorphin gene, any neurotensin (1, 2, or 3) gene, the Bdnf gene, the TD02 gene, the Sgk gene, the Fkbp5&4 gene, the Edg2 gene, the Id2 gene, the Gabl gene, and the Fgfr2 gene informs adjusting dosage of (−)-Hydroxycitric acid (HCA) also administered to the patient as part of the method; or
r. the determined genotype comprises allelic analysis of polymorphisms in a gene selected from the group consisting of the Hpcall gene, the COMT gene, the NET gene, and the MAOA gene informs adjusting dosage of Pyridoxal phosphate also administered to the patient as part of the method;
s. the determined genotype comprises allelic analysis of polymorphisms in a gene selected from the group consisting of the HpcaII gene and any ATPase gene informs adjusting dosage of magnesium also administered to the patient as part of the method; or
t. the determined genotype comprises allelic analysis of polymorphisms in a gene selected from the group consisting of the leptin receptor gene, any of the dopamine DI-5 genes, the HpcaII gene, the HTT gene, the HTRIA gene, the HTRID gene, the HTR2A gene, the HTR2c gene, they-HT-2A gene, the 5-HT 2B gene, the 5-HT-4 gene, the 5-HT-7 gene, the ANKKI gene, the DATI gene, the DBH gene, and the TD02 gene informs adjusting dosage of potassium also administered to the patient as part of the method.

75. A method according to claim 66 that further comprises administering at least one or more additional substances selected from the group consisting of (−)-Hydroxycitric acid (HCA), Passion flower (Passiflora incarnata) L Extract, Potassium, Thiamin, Vitamin B₅, and Calcium, wherein the additional substance(s) is(are) optionally each administered in a daily dosage ranging from approximately 1 meg to 30,000 mg.

76. A method according to claim 66 that further comprises administering a pain-alleviating ointment formulation, wherein the ointment formulation optionally comprises a base ointment cream comprising a solubilizer, wherein the solubilizer optionally is selected from the group consisting of a soya-lecithin aggregate; a micronized, cyclic monoterpene; a cyclohexanone derivative; isosorbide dinitrate; and Lipoderm.

77. A pain-alleviating ointment formulation for use in practicing a method according to claim 76, wherein the ointment formulation is selected from the group consisting of:
a. an ointment formulation comprising D-phenylalanine, wherein the ointment formulation optionally is selected from the group consisting of D-phenylalanine, LID, GBP, KET, KEPF (optionally in the ratio of about 10/5/10/10/10%); D-Phenylanine, GBP, KET, BAC (optionally in the ratio of about 10/10/10/4%); D-Phenylalanine GBP, KET, LID (optionally in the ratio of about 10/6/10/10%); D-Phenylanine, GBP, KET, AM, BAC (optionally in the ratio of about 10/6/6/4/4%); D-Phenylalanine, KEPF (optionally in the ratio of about 10/10%); D-Phenylalanine, KEPF (optionally in the ratio of about 10/20%); D-Phenylalanine, KEPF, LID (optionally in the ratio of about 10/10/5%); D-Phenylalanine, KEPF, CLB(optionally in the ratio of about 10/20/2%); D-Phenylalanine, KEPF, LID, CLB (optionally in the ratio of about 10/20/5/2%); D-Phenylalanine, IBUF, KEPF, CLB (optionally in the ratio of about 10/10/10/1%); D-Phenylalanine, LID (optionally in the ratio of about 10/10%); D-Phenylalanine, DICLO (optionally in the ratio of about 10/10%); D-phenylalanine, CAP, MT, CAMP (optionally in the ratio of about 10/0.0375%); D-phenylalanine, CAP, MT, CAMP (optionally in the ratio of about 10/05%); and D-phenylalanine KEPF, KET, CAP (optionally in the ratio of about 10/10/6/0.075%);
b. an ointment formulation comprising L-phenylalanine, wherein the ointment formulation optionally is selected from the group consisting of L-phenylalanine, LID, GBP, KET, KEPF (optionally in the ratio of about 10/5/10/10/10%); L-Phenylanine, GBP, KET, BAC (optionally in the ratio of about 10/10/10/4%); L-Phenylalanine GBP, KET, LID (optionally in the ratio of about 10/6/10/10%); L-Phenylanine, GBP, KET, AM, BAC (optionally in the ratio of about 10/6/6/4/4%); L-Phenylalanine, KEPF (optionally in the ratio of about 10/10%); L-Phenylalanine, KEPF (optionally in the ratio of about 10/20%); L-Phenylalanine, KEPF, LID (optionally in the ratio of about 10/10/5%); L-Phenylalanine, KEPF, CLB (optionally in the ratio of about 10/20/2%); L-Phenylalanine, KEPF, LID, CLB (optionally in the ratio of about 10/20/5/2%); L-Phenylalanine, IBUF, KEPF, CLB (optionally in the ratio of about 10/10/10/1%); L-Phenylalanine, LID (optionally in the ratio of about 10/10%); L-Phenylalanine, DICLO (optionally in the ratio of about 10/10%); L-phenylalanine, CAP, MT, CAMP (optionally in the ratio of about 10/0.0375%); L-phenylalanine, CAP, MT, CAMP (optionally in the ratio of about 10/05%); L-phenylalanine, KEPF, KET, CAP (optionally in the ratio of about 10/10/6/0.075%);
c. an ointment formulation comprising L-Glutamine, wherein the ointment formulation optionally is selected from the group consisting of L-Glutamine, LID, GBP, KET, KEPF (optionally in the ratio of about 10/5/10/10/10%); L-Glutamine, GBP, KET, BAC (optionally in the ratio of about 10/10/10/4%); L-Glutamine, GBP, KET, LID (optionally in the ratio of about 10/6/10/10%); L-Glutamine, GBP, KET, AM, BAC (optionally in the ratio of about 10/6/6/4/4%); L-Glutamine, KEPF (optionally in the ratio of about 10/10%); L-Glutamine, KEPF (optionally in the ratio of about 10/20%); L-Glutamine, KEPF, LID (optionally in the ratio of about 10/10/5%); L-Glutamine, KEPF, CLB (optionally in the ratio of about 10/20/2%); L-Glutamine, KEPF, LID, CLB (optionally in the ratio of about 10/20/5/2%); L-Glutamine IBUF, KEPF, CLB (optionally in the ratio of about 10/10/10/1%); L-Glutamine, LID (optionally in the ratio of about 10/10%); L-Glutamine, DICLO (optionally in the ratio of about 10/10%); L-Glutamine, CAP, MT, CAMP (optionally in the ratio of about 10/0.0375%); L-Glutamine, CAP, MT, CAMP (optionally in the ratio of about 10/05%); L-Glutamine KEPF, KET, CAP (optionally in the ratio of about 10/10/6/0.075%);
d. an ointment formulation comprising 5-HTP, wherein the ointment formulation optionally is selected from the group consisting of 5-HTP, LID, GBP, KET, KEPF (optionally in the ratio of about 10/5/10/10/10%); 5-HTP, GBP, KET, BAC (optionally in the ratio of about 10/10/10/4%); 5-HTP GBP, KET, LID (optionally in the ratio of about 10/6/10/10%); 5-HTP, GBP, KET, AM, BAC (optionally in the ratio of about 10/6/6/4/4%); 5-HTP, KEPF (optionally in the ratio of about 10/10%); 5-HTP, KEPF (optionally in the ratio of about 10/20%); 5-HTP, KEPF, LID (optionally in the ratio of about 10/10/5%); 5-HTP, KEPF, CLB (optionally in the ratio of about 10/20/2%); 5-HTP, KEPF, LID, CLB(optionally in the ratio of about 10/20/5/2%); 5-HTP, IBUF, KEPF, CLB (optionally in the ratio of about 10/10/10/1%); 5-HTP, LID (optionally in the ratio of about 10/10%); 5-HTP, DICLO (optionally in the ratio of about 10/10%); 5-HTP, CAP, MT, CAMP (optionally in the ratio of about 10/0.0375%); 5-HTP, CAP, MT, CAMP (optionally in the ratio of about 10/05%); 5-HTP, KEPF, KET, CAP (optionally in the ratio of about 10/10/6/0.075%);
e. an ointment formulation comprising Rhodiola rosea, wherein the ointment formulation optionally is selected from the group consisting of Rhodiola rosea, LID, GBP, KET, KEPF (optionally in the ratio of about 10/5/10/10/10%); Rhodiola rosea, GBP, KET, BAC (optionally in the ratio of about 10/10/10/4%); Rhodiola rosea GBP, KET, LID (optionally in the ratio of about 10/6/10/10%); Rhodiola rosea, GBP, KET, AM, BAC (optionally in the ratio of about 10/6/6/4/4%); Rhodiola rosea, KEPF (optionally in the ratio of about 10/10%); Rhodiola rosea, KEPF (optionally in the ratio of about 10/20%); Rhodiola rosea, KEPF, LID (optionally in the ratio of about 10/10/5%); Rhodiola rosea, KEPF, CLB (optionally in the ratio of about 10/20/2%); Rhodiola rosea, KEPF, LID, CLB (optionally in the ratio of about 10/20/5/2%); Rhodiola rosea IBUF, KEPF, CLB (optionally in the ratio of about 10/10/10/1%); Rhodiola rosea, LID (optionally in the ratio of about 10/10%); Rhodiola rosea, DICLO (optionally in the ratio of about 10/10%); Rhodiola rosea, CAP, MT, CAMP (optionally in the ratio of about 10/0.0375%); Rhodiola rosea, CAP, MT, CAMP (optionally in the ratio of about 10/05%); Rhodiola rosea, KEPF, KET, CAP (optionally in the ratio of about 10/10/6/0.075%);
f. an ointment formulation comprising chromium salt, wherein the ointment formulation optionally is selected from the group consisting of chromium salt, LID, GBP, KET, KEPF (optionally in the ratio of about 0.01/5/10/10/10%); chromium salt, GBP, KET, BAC (optionally in the ratio of about 0.01/10/10/4%); chromium salt GBP, KET, LID (optionally in the ratio of about 0.01/6/10/10%); chromium salt, GBP, KET, AM, BAC (optionally in the ratio of about 0.01/6/6/4/4%); chromium salt, KEPF (optionally in the ratio of about 0.01/10%); chromium salt, KEPF (optionally in the ratio of about 0.01/20%); chromium salt, KEPF, LID (optionally in the ratio of about 0.01/10/5%); chromium salt, KEPF, CLB (optionally in the ratio of about 0.01/20/2%); chromium salt, KEPF, LID, CLB (optionally in the ratio of about 0.01/20/5/2%); chromium salt, IBUF, KEPF, CLB (optionally in the ratio of about 0.01/10/10/1%); chromium salt, LID (optionally in the ratio of about 0.01/10%); chromium salt, DICLO(optionally in the ratio of about 0.01/10%); chromium salt, CAP, MT, CAMP (optionally in the ratio of about 0.01/0.0375%); chromium salt, CAP, MT, CAMP (optionally in the ratio of about 0.01/05%); chromium salt, KEPF, KET, CAP (optionally in the ratio of about 0.01/10/6/0.075%);
g. an ointment formulation comprising Pyridoxal-phosphate, wherein the ointment formulation optionally is selected from the group consisting of Pyridoxal-phosphate, LID, GBP, KET, KEPF (optionally in the ratio of about 0.05/5/10/10/10%); Pyridoxal-phosphate, GSP, KE T, BAC (optionally in the ratio of about 0.05/10/10/4%); Pyridoxal-phosphate, GSP, KET, LID (optionally in the ratio of about 0.01/6/10/10%); Pyridoxal-phosphate, GBP, KET, AM, BAC (optionally in the ratio of about 0.05/6/6/4/4%); Pyridoxal-phosphate, KEPF (optionally in the ratio of about 0.05/10%); Pyridoxal-phosphate, KEPF (optionally in the ratio of about 0.05/20%); Pyridoxal-phosphate, KEPF, LID (optionally in the ratio of about 0.05/10/5%); Pyridoxal-phosphate, KEPF, CLB (optionally in the ratio of about 0.05/20/2%); Pyridoxal-phosphate, KEPF, LID, CLB(optionally in the ratio of about 0.01/20/5/2%); Pyridoxal-phosphate IBUF, KEPF, CLB (optionally in the ratio of about 0.01/10/10/1%); Pyridoxal-phosphate, LID (optionally in the ratio of about 0.01/10%); Pyridoxal-phosphate, DICLO(optionally in the ratio of about 0.05/10%); Pyridoxal-phosphate, CAP, MT, CAMP (optionally in the ratio of about 0.05/0.0375%); Pyridoxal-phosphate, CAP, MT, CAMP (optionally in the ratio of about 0.05/05%); and Pyridoxal-phosphate, KEPF, KET, CAP (optionally in the ratio of about 0.05/10/6/0.075%);
h. an ointment formulation comprising L-Tyrosine, wherein the ointment formulation optionally is selected from the group consisting of L-Tyrosine, LID, GBP, KET, KEPF (optionally in the ratio of about 10/5/10/10/10%); L-Tyrosine, GBP, KET, BAC (optionally in the ratio of about 10/10/10/4%); L-Tyrosine GBP, KET, LID (optionally in the ratio of about 10/6/10/10%); L-Tyrosine, GBP, KET, AM, BAC (optionally in the ratio of about 10/6/6/4/4%); L-Tyrosine, KEPF (optionally in the ratio of about 10/10%); L-Tyrosine, KEPF (optionally in the ratio of about 10/20%); L-Tyrosine, KEPF, LID (optionally in the ratio of about 10/10/5%); L-Tyrosine, KEPF, CLB(optionally in the ratio of about 10/20/2%); L-Tyrosine, KEPF, UD, CLB (optionally in the ratio of about 10/20/5/2%); L-Tyrosine, IBUF, KEPF, CLB (optionally in the ratio of about 10/10/10/1%); L-Tyrosine, LID (optionally in the ratio of about 10/10%); L-Tyrosine DICLO (optionally in the ratio of about 10/10%); L-Tyrosine, CAP, MT, CAMP (optionally in the ratio of about 10/0.0375%); L-Tyrosine, CAP, MT, CAMP (optionally in the ratio of about 10/05%); and L-Tyrosine, KEPF, KET, CAP (optionally in the ratio of about 10/10/6/0.075%);
i. an ointment formulation comprising Synaptamine, wherein the ointment formulation optionally is selected from the group consisting of Synaptamine, LID, GBP, KET, KEPF (optionally in the ratio of about 10/5/10/10/10%); Synaptamine, GBP, KET, BAC (optionally in the ratio of about 10/10/10/4%); Synaptamine, GBP, KET, LID (optionally in the ratio of about 10/6/10/10%); Synaptamine, GBP, KET, AM, BAC (optionally in the ratio of about 10/6/6/4/4%); Synaptamine, KEPF (optionally in the ratio of about 10/10%); Synaptamine, KEPF (optionally in the ratio of about 10/20%); Synaptamine, KEPF, LID (optionally in the ratio of about 10/10/5%); Synaptamine, KEPF, CLB (optionally in the ratio of about 10/20/2%); Synaptamine, KEPF, LID, CLB (optionally in the ratio of about 10/20/5/2%); Synaptamine, IBUF, KEPF, CLB (optionally in the ratio of about 10/10/10/1%); Synaptamine, LID (optionally in the ratio of about 10/10%); Synaptamine DICLO (optionally in the ratio of about 10/10%); Synaptamine, CAP, MT, CAMP (optionally in the ratio of about 10/0.0375%); Synaptamine, CAP, MT, CAMP (optionally in the ratio of about 10/05%); and Synaptamine, KEPF, KET, CAP (optionally in the ratio of about 10/10/6/0.075%);
j. an ointment formulation comprising Kyotorphin, wherein the ointment formulation optionally is selected from the group consisting of Kyotorphin, Synaptamine, LID, GBP, KET, KEPF (optionally in the ratio of about 10/5/10/10/10%); Kyotorphin, Synaptamine, GBP, KET, BAC (optionally in the ratio of about 10/10/10/4%); Kyotorphin, Synaptamine, GBP, KET, LID (optionally in the ratio of about 10/6/10/10%); Kyotorphin, Synaptamine, KEPF (optionally in the ratio of about 10/10%); Kyotorphin, Synaptamine, KEPF (optionally in the ratio of about 10/20%); Kyotorphin, Synaptamine, KEPF, LID (optionally in the ratio of about 10/10/5%); Kyotorphin, Synaptamine, KEPF, CLB (optionally in the ratio of about 10/20/2%); Kyotorphin, Synaptamine, KEPF, LID, CLB (optionally in the ratio of about 10/20/5/2%); Kyotorphin, Synaptamine, IBUF, KEPF, CLB (optionally in the ratio of about 10/10/10/1%); Kyotorphin, Synaptamine, LID (optionally in the ratio of about 10/10%); Kyotorphin Synaptamine DICLO (optionally in the ratio of about 10/10%); Kyotorphin, Synaptamine, CAP, MT, CAMP (optionally in the ratio of about 10/0.0375%); Kyotorphin Synaptamine, CAP, MT, CAMP (optionally in the ratio of about 10/05%); and Kyotorphin, Synaptamine KEPF, KET, CAP (optionally in the ratio of about 10/10/6/0.075%); and
k. an ointment formulation comprising Kyotorphin, wherein the ointment formulation optionally is selected from the group consisting of Kyotorphin, LID, GBP, KET, KEPF (optionally in the ratio of about 10/5/10/10/10%); Kyotorphin, GBP, KET, BAC (optionally in the ratio of about 10/10/10/4%); Kyotorphin, GBP, KET, LID (optionally in the ratio of about 10/6/10/10%); Kyotorphin, GBP, KET, AM, BAC (optionally in the ratio of about 10/6/6/4/4%); Kyotorphin KEPF (optionally in the ratio of about 10/10%); Kyotorphin, KEPF (optionally in the ratio of about 10/20%); Kyotorphin, KEPF, LID (optionally in the ratio of about 10/10/5%); Kyotorphin KEPF, CLB (optionally in the ratio of about 10/20/2%); Kyotorphin, KEPF, LID, CLB (optionally in the ratio of about 10/20/5/2%); Kyotorphin IBUF, KEPF, CLB (optionally in the ratio of about 10/10/10/1%); Kyotorphin, LID (optionally in the ratio of about 10/10%); Kyotorphin DICLO (optionally in the ratio of about 10/10%); Kyotorphin, CAP, MT, CAMP (optionally in the ratio of about 10/0.0375%); Kyotorphin, CAP, MT, CAMP (optionally in the ratio of about 10/05%); and Kyotorphin, KEPF, KET, CAP (optionally in the ratio of about 10/10/6/0.075%).
